(19) United States
(12) Patent Application Publication
(10) Pub. No.: US 2022/0040230 A1
(43) Pub. Date: Feb. 10, 2022

(54) COMPOSITIONS AND METHODS FOR IMMUNOTHERAPIES

(71) Applicant: The Regents of the University of California, Oakland, CA (US)

(72) Inventor: Anahid Jewett, Los Angeles, CA (US)

(21) Appl. No.: 17/299,220

(22) PCT Filed: Dec. 10, 2019
(86) PCT No.: PCT/US19/65381

§ 371 Date: Jun. 2, 2021

Related U.S. Application Data
(60) Provisional application No. 62/778,189, filed on Dec. 11, 2018.

(51) Int. Cl. A61K 35/17; A61K 39/395; A61K 36/03; A61K 35/741; A61K 35/744; A61K 35/745; A61K 35/747; A61K 31/337; A61K 33/243; A61K 31/198; A61P 35/00

(52) U.S. Cl. CPC A61K 35/17; A61K 39/39558; A61K 39/3955; A61K 36/03; A61K 35/741; A61P 35/00; A61K 35/745; A61K 35/747; A61K 31/337; A61K 33/243; A61K 31/198; A61K 35/744

(57) ABSTRACT

The present application relates to pharmaceutical compositions comprising multiple cell types, and methods of using these compositions to treat cancer in a subject.

### CROSS-REFERENCE TO RELATED APPLICATIONS

This application claims the benefit of U.S. Provisional Application No. 62/778,189, filed on Dec. 11, 2018, the entire contents of which are incorporated herein in their entirety by this reference.

### BACKGROUND OF THE INVENTION

The lymphocyte population in peripheral blood mononuclear cells (PBMCs) mainly constitutes T-cells, B-cells and, the natural-killer cells (NK cells). NK cells are known to play central defense against viral infection and killing tumor cells, and have been classified as effectors of innate immunity due to the lack of antigen specific cell surface receptors. T cells are known to mediate the cellular immunity mediating humoral immunity, provide adaptive immunity which work in close collaboration with the innate immune system. Human NK cells are defined phenotypically by the surface expression of CD56 and CD16, and by their lack of CD3 surface expression. About 90% of human NK cells are CD56dim CD16bright cells and found to be the major cytotoxic subset, whereas CD56bright CD16dim/-NK cells were found to secrete more cytokines. Major cytokines, secreted by NK cells are interferon-gamma (IFN-γ), tumor necrosis factor-alpha (TNF-α), TNF-β, granulocyte macrophage-colony stimulating factor (GM-CSF), interleukin-10 (IL-10), and IL-13.

NK cells isolated from the peripheral blood of cancer patients display phenotypic and functional alterations especially during advanced stage of cancer. It has been shown that freshly isolated tumor infiltrating NK cells are not cytotoxic to autologous tumors. T cells dysfunction has also been reported in cancer patients. Moreover, NK and T cells, especially NK cells obtained from the peripheral blood of patients with cancer have significantly reduced function particularly cytotoxic activity. Suppression of NK cells is mediated by downregulation of NK receptors in the tumor microenvironment. NK cells infiltration and cytotoxic activity of peripheral-blood lymphocytes has indirect co-relation the prognosis of cancer patients.

The major T-cell subpopulations are helper (CD4+) and cytotoxic (CD8+) T cells. The cellular immune responses that protect against tumors typically have been attributed to CD8+ T cells, CD8+ T cells are associated with chemo-response against the cancer. High numbers of T cells with CD8+ memory T cells, decreased proportions of tumor-infiltrating CD4+ T cells with high percentages of T-regulatory (Tregs) and, reversed CD4/CD8 ratios at tumor site were significantly associated with overall survival in patients with solid cancers. It has been shown that CD45RA+ T cells with high expression of CD62L and CCR7 have longer active life-span and are more effective against cancers in comparison to T memory cells. CD28 co-stimulation play crucial role in T cells anti-tumor and anti-microbial activity, lower surface expression of CD28 on cancer patients' T cells indicate their lower activity of T cells to fight against the cancer and the infection in those patients. Lower surface expression of CD127 on the surface of T cells has been shown to be influenced by the presence of cancer and infections.

Natural killer (NK) cells lyse and differentiate cancer stem cells/undifferentiated tumors with lower expression of MHC class I, CD54 and B7H1 and higher expression of CD44. Medium and high cytotoxic activity of peripheral-blood lymphocytes are associated with reduced cancer risk, and high NK-cell infiltration of the tumor is associated with a better prognosis, whereas low activity is associated with increased cancer risk.

Lower MHC-class I expression on cancer stem cells (CSCs)/poorly differentiated tumors might favor their survival, and explain their limited effectiveness to T-cell based immunotherapies in cancer patients. CSCs are excellent targets of NK cell-mediated cytotoxicity, whereas their differentiated counterparts are significantly more resistant. Furthermore, de-differentiation of tumors resulted in their increased susceptibility to NK cell-mediated cytotoxicity. It is known that cytotoxic function of primary NK cells is suppressed after their interaction with CSCs/stem cells. NK cells, as a result of CD16 receptor cross-linking or interaction with CSCs/undifferentiated tumors, undergo split-anergy, a key event in which NK-cytotoxicity is lost but a greater secretion of IFN-γ is triggered which promote an increase in the differentiation antigen expression of MHC-class I, CD54 and PD-L1 on tumors which has recently been shown to correlate with effectiveness of anti-PD-1 therapy. Indeed, overall higher levels of circulating NK cells are associated with better prognosis in cancer patients. However, NK cell cytotoxic activity in peripheral blood of cancer patients is reduced, and also the expression of NK cell activating receptors were diminished even at the early stages of cancer and are further reduced in advanced disease. Defect in NK cell function is seen both at the pre-neoplastic and neoplastic stages of pancreatic cancer. Among pancreatic tumors, MiaPaCa-2 (MP2) pancreatic cancer stem cells (CSCs) were shown to have increased tumor cell growth, migration, clonogenicity, and self-renewal capacity and chemotherapy resistance.

Immunotherapy with a single type of immune cells, although effective, has not demonstrated complete eradication of tumors, because it utilizes only a specialized subset of the immune cells to target a subpopulation of cancer cells. Thus, there is a great need to identify and develop therapeutic compositions and methods for improved immunotherapies that encompass multiple immune cell types that can target cancer cells using multiple mechanisms.

### SUMMARY OF THE INVENTION

The present invention is based, at least in part, on the discovery that primary NK cells mediate antibody-dependent cellular cytotoxicity (ADCC) against differentiated tumors but not against undifferentiated/stem-like tumors, and that super-charged NK cells do not mediate ADCC but can target/kill differentiated tumors directly. The present invention is also based, at least in part, on the discovery that NK cells expand CD8+ T cells preferentially, and that NK cells prevent progression of cancer through selection and differentiation of CSCs/poorly-differentiated tumors, resulting in inhibition of the tumor aggressiveness, metastatic potential, and increased susceptibility to chemotherapy.

In one aspect, the invention provides a method of treating a subject afflicted with a cancer, comprising administering to the subject an immunological composition, wherein the immunological composition comprises at least two cell types selected from: (a) allogeneic primary NK cells, (b) allogeneic super-charged NK cells, (c) autologous super-charged NK cell expanded CD8+ T cells, and (d) allogeneic super-charged NK cell expanded autologous CD8+ T cells, is provided.

Numerous embodiments are further provided that can be applied to any aspect of the present invention and/or combined with any other embodiment(s) described herein. For example, in certain embodiments, the method comprises administering to the subject an immunological composition comprising three cell types, or even four cell types. In certain embodiments, the NK cells of the subject show one or more reduced activities selected from: (a) cytokine secretion, optionally wherein the cytokine is IFN-γ, (b) cytotoxicity, (c) expansion of CD8+ T cells, (d) differentiation of stem-like/poorly differentiated tumor cells, and (e) ADCC activity. In certain embodiments, the immunological composition is administered in a pharmaceutically acceptable formulation. In certain embodiments, the method further comprises administering to the subject an antibody against at least one surface protein that is highly expressed on cancer cells, e.g., an antibody that binds MICA/MICB. The antibody may be administered in an amount sufficient to induce ADCC.

In certain embodiments, the method further comprises activating NK cells by inducing or enhancing secretion of IFN-γ in the NK cells, e.g., by administering to the subject one or more additional agents that enhance secretion of IFN-γ by the NK cells, such as IL-2, anti-CD16 antibody, anti-CD3 antibody, anti-CD28 antibody, Mekabu, and/or a composition comprising at least one bacterial strain (e.g., Streptococcus thermophiles, Bifidobacterium longum, Bifidobacterium breve, Bifidobacterium infantis, Lactobacillus acidophilus, Lactobacillus plantarum, Lactobacillus paracasei, KE99, or Lactobacillus bulgaricus), optionally wherein the at least one bacterial strain is either alive or sonicated. In certain preferred embodiments, the composition comprises AJ2 bacteria. In particularly preferred embodiments, the one or more additional agents that activate NK cells are Mekabu and AJ2 bacteria.

In certain embodiments, the method further comprises administering to the subject at least one additional immunotherapy and/or cancer therapy, e.g., which may be administered before, after, or concurrently with the immunological composition. In certain such embodiments, the at least one additional immunotherapy inhibits an immune checkpoint, such as CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 or A2aR. In certain preferred embodiments, the immune checkpoint is selected from CTLA-4, PD-1, PD-L1, and PD-L2. In certain other embodiments, the cancer therapy is selected from radiation, a radiosensitizer, a chemotherapy, interferon, and an interferon-inducing agent. In certain such embodiments, the cancer therapy is a chemotherapy, optionally wherein the chemotherapy is paclitaxel and/or cisplatin.

In certain embodiments, the method further comprises administering to the subject an agent that induces differentiation of poorly differentiated cancer cells, optionally wherein the agent is N-acetylcysteine (NAC).

In certain embodiments, the cancer is pancreatic cancer, or oral cancer, e.g., oral squamous carcinoma. In certain embodiments, the cancer is highly differentiated. In other embodiments, the cancer is stem-like/poorly differentiated.

In certain embodiments, the subject is a mammal, e.g., a mouse or a human, preferably a human.

In another aspect, the invention provides a method of killing or inhibiting proliferation of cancer cells, comprising contacting the cancer cells with an immunological composition (e.g., a composition as described herein), e.g., wherein the immunological composition comprises at least two cell types selected from: (a) allogeneic primary NK cells, (b) allogeneic super-charged NK cells, (c) autologous super-charged NK cell expanded CD8+ T cells, and (d) allogeneic super-charged NK cell expanded autologous CD8+ T cells.

As described above, numerous embodiments are further provided that can be applied to any aspect of the present invention and/or combined with any other embodiment(s) described herein. For example, in certain embodiments, the method comprises contacting the cancer cells with an immunological composition comprising at least three of these cell types, or even four of these cell types. The immunological composition may be in pharmaceutically acceptable formulation.

In certain embodiments, the method further comprises contacting the cancer cells with an antibody against at least one surface protein that is highly expressed on cancer cells, e.g., an antibody that binds MICA/MICB. In certain embodiments, the antibody is provided in an amount sufficient to induce ADCC. In certain embodiments, the method further comprises activating NK cells by inducing or enhancing secretion of IFN-γ in the NK cells, e.g., by contacting the NK cells with one or more additional agents that enhance secretion of IFN-γ by the NK cells, such as IL-2, anti-CD16 antibody, anti-CD3 antibody, anti-CD28 antibody, Mekabu, and a composition comprising at least one bacterial strain (such as Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus), optionally wherein the at least one bacterial strain is either alive or sonicated. In certain preferred embodiments, the composition comprises AJ2 bacteria. In particularly preferred embodiments, the one or more additional agents are Mekabu and AJ2 bacteria.

In certain embodiments, the method further comprises contacting the cancer cells with at least one additional immunotherapy and/or cancer therapy, e.g., which may be added before, after, or concurrently with the immunological composition. In certain embodiments, the at least one additional immunotherapy inhibits an immune checkpoint, such as CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 or A2aR. In certain preferred embodiments, the immune checkpoint is selected from CTLA-4, PD-1, PD-L1, and PD-L2. In certain embodiments, the cancer therapy is selected from radiation, a radiosensitizer, a chemotherapy, interferon, and an interferon-inducing agent. In certain such embodiments, the cancer therapy is a chemotherapy, optionally wherein the chemotherapy is a paclitaxel and/or cisplatin. In certain embodiments, the method further comprises contacting the cancer cells with an agent that induces differentiation of poorly differentiated cancer cells, optionally wherein the agent is N-acetylcysteine (NAC).

In certain embodiments, the cancer is pancreatic cancer, or oral cancer, e.g., oral squamous carcinoma. In certain embodiments, the cancer is highly differentiated. In other embodiments, the cancer is stem-like/poorly differentiated.

In certain embodiments, the subject is a mammal, e.g., a mouse or a human, preferably a human.

In another aspect, the invention provides an immunological composition capable of eliciting an immune response in a subject, comprising at least two cell types selected from: (a) allogeneic primary NK cells, (b) allogeneic super-charged NK cells, (c) autologous super-charged NK cell expanded CD8+ T cells, and (d) allogeneic super-charged NK cell expanded autologous CD8+ T cells.

In certain such embodiments, the immunological composition comprises three cell types, or even four cell types. The immunological composition may be a pharmaceutically acceptable formulation. In certain embodiments, the immunological composition further comprises an antibody against at least one surface protein that is highly expressed on cancer cells, such as an antibody that binds MICA/MICB. The antibody may be present in an amount sufficient to induce ADCC when administered to a subject.

In certain embodiments, the immunological composition further comprises one or more additional agents that enhance secretion of IFN-γ by the NK cells, such as IL-2, anti-CD16 antibody, anti-CD3 antibody, anti-CD28 antibody, Mekabu, or a composition comprising at least one bacterial strain (e.g., Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus), optionally wherein the at least one bacterial strain is either alive or sonicated. In certain preferred embodiments, the composition comprises AJ2 bacteria. In particularly preferred embodiments, the immunological composition further comprises Mekabu and AJ2 bacteria. In certain embodiments, the immunological composition further comprises at least one additional immunotherapy and/or cancer therapy. In certain such embodiments, the at least one additional immunotherapy inhibits an immune checkpoint, such as CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 or A2aR. In yet certain preferred embodiments, the immune checkpoint is selected from CTLA-4, PD-1, PD-L1, and PD-L2. In certain embodiments, the immunological composition further comprises a cancer therapy selected from a radiosensitizer, a chemotherapy, interferon, and an interferon-inducing agent. In certain such embodiments, the cancer therapy is chemotherapy, optionally wherein the chemotherapy is paclitaxel and/or cisplatin. In certain embodiments, the immunological composition further comprises an agent that induces differentiation of poorly differentiated cancer cells, optionally wherein the agent is N-acetylcysteine (NAC).

### BRIEF DESCRIPTION OF THE DRAWINGS

FIG. 1A-FIG. 1D show that differentiation stage of oral tumor cells correlates with sensitivity to NK cell-mediated lysis. The OSCSCs and MP2 were differentiated as explained in methods and material. The surface expression of CD44, MHC-I, and MICA on OSCSCs, OSCCs and split-anergized NK cell supernatant-differentiated OSCSCs tumor cells (FIG. 1A), and MP2, PL12, and differentiated MP2 (FIG. 1B) was assessed using flow cytometric analysis after staining with respective PE-conjugated antibodies. Isotype control antibodies were used as controls. NK-cell mediated cytotoxicity was determined using a standard 4-hour 51Cr release assay against OSCCs, differentiated OSCCS and OSCSCs (FIG. 1C), and MP2, differentiated MP2, and PL12 (FIG. 1D) tumor cells. Purified NK cells (1×10⁶ cells/ml) were treated with IL-2 (1000 U/ml) for 18 hours before they were added to ⁵¹Cr labeled tumor cells at various effector to target ratios.

FIG. 2 shows that differentiated oral and pancreatic tumor cells expressed higher MICA/MICB surface expression in comparison to their undifferentiated compartments. The OSCSCs and MP2 were differentiated as explained in methods and material. The surface expression of MICA/MICB on OSCCS, OCSCSs, and split-anergized NK cell supernatant-differentiated MP2, PL12 and split-anergized NK cell supernatant-differentiated OSCSCs (top) PL12, MP2 and NK cell supernatant-differentiated MP2 (bottom) was assessed using flow cytometric analysis after staining with respective PE-conjugated antibodies. Isotype control antibodies were used as controls.

FIG. 3A-FIG. 3E show that antibodies specific to MICA/MICB increased NK cell-mediated ADCC against OSCCs, while OSCSCs cells were not targeted significantly through ADCC. Freshly purified NK cells from healthy donors were left untreated, treated with IL-2 (1,000 U/mL) or combination of IL-2 and anti-CD16 mAb (3 μg/ml), for 18 hours. OSCCS, and OSCSCs were labeled with ⁵¹Cr, and then left untreated or treated with anti-MICA/MICB antibody (5 μg/ml) for 30 minutes. The unbounded antibodies were washed and the cytotoxicity against the OSCCs (FIG. 3A) and OSCSCs (FIG. 3B) untreated or treated with the antibody against MICA/MICB was determined using the standard 4-hour ⁵¹Cr release assay (FIGS. 3A and 3B are representative of one study). The ADCC induced fold increase in cytotoxicity of untreated (FIG. 3C), IL-2 treated (FIG. 3D) and the IL-2+anti-CD16 mAb (FIG. 3E) treated NK cells were measured.

FIG. 4A-FIG. 4E show that antibodies specific to MICA/MICB increased NK cell-mediated ADCC against PL12, while MP2 cells were not targeted. Freshly purified NK cells from healthy donors were left untreated, treated with IL-2 (1000 U/mL) or combination of IL-2 and anti-CD16 mAb (3 μg/mL), for 18 hours. PL12, and MP2 were labeled with ⁵¹Cr, and then left untreated or treated with anti-MICA/MICB antibody (5 μg/ml) for 30 minutes. The unbounded antibodies were washed and the cytotoxicity against the PL12 (FIG. 4A) and MP2 (FIG. 4B) untreated or treated with the antibody against MICA/MICB was determined using the standard 4-hours ⁵¹Cr release assay. The ADCC induced fold increase in cytotoxicity for untreated (FIG. 4C), IL-2 treated (FIG. 4D), and the IL-2+anti-CD16 mAb treated (FIG. 4E) NK cells were measured.

FIG. 5A-FIG. 5F show that differentiation of OSCSCs with split-anergized NK cells supernatants results in their susceptibility to NK cell-mediated ADCC through anti MICA/MICB antibody. OSCSCs and MP2 tumor cells were differentiated as described in methods and material section. Freshly purified NK cells from healthy donors were left untreated or treated with IL-2 (1,000 U/ml) for 18 hours. OSCCS, differentiated OSCSCs, and OSCSCs, were labeled with ⁵¹Cr, and then left untreated or treated with antiMICA/MICB antibody (5 μg/ml) for 30 minutes. The unbounded antibodies were washed and the cytotoxicity of untreated NK cells and/or IL-2 treated against untreated or antiMICA/MICB treated the OSCCs (FIG. 5A), differentiated OSCSCs (FIG. 5B), OSCSCs (FIG. 5C), PL12 (FIG. 5D), differentiated MP2 (FIG. 5E), and MP2 (FIG. 5F) were determined using the standard 4-hours ⁵¹Chromium release assay.

FIG. 6A-FIG. 6C show that antibodies specific to MICA/MICB increased IFN-γ secretion by NK cells when cultured with differentiated Oral tumors expressing MICA/MICB. OSCCs and OSCSCs were cultured in absence or presence of anti-MICA/MICB Ab (5 μg/mL) overnight. The unbounded antibodies were washed. Freshly purified NK cells from healthy donors were left untreated, treated with IL-2 (1,000 U/mL) or combination of IL-2 and anti-CD16 mAb (3 μg/mL), for 18 hours and they were co-cultured with OSCCs (FIG. 6A), and OSCSCs (FIG. 6B). After 24 hours the supernatant was collected from the cultures and IFN-γ was measured with ELISA. Figure A and B are representative of one experiment. FIG. 6C shows the IFN-γ level of IL-2 treated NK co-cultured with untreated or anti-MICA/MICB antibody treated OSCCs and OSCSCs in three different experiments.

FIG. 7A-FIG. 7B shows that expanded NK cells target both undifferentiated and differentiated tumor cells while primary NK cells preferentially target undifferentiated/Stem like population. NK cells were purified from healthy donors' blood and expanded as described in methods and material section. After day 15 of expansion, primary NK cells were purified from the same donor (1×10⁶ cells/mL) and treated with IL-2 (1,000 U/mL) and expanded NK cells reactivated with IL-2 (1,000 U/mL) for 18 hours. OSCCS and OSCSCs were labeled with ⁵¹Cr, and the cytotoxicity of primary and expanded NK cells against OSCCs (left) and OSCSCs (right) were determined in three different experiment, using the standard 4-hours ⁵¹Cr release assay (FIG. 7A). PL12 and MP2 were labeled with ⁵¹Cr, the cytotoxicity of primary NK cells, and cytotoxicity of expanded NK against MP2 and PL12 was determined (FIG. 7B).

FIG. 8A-FIG. 8C show that combination of IL-2 and antiCD16 treatment induces split anergy in primary NK cells but not in super-charged NK cells. NK cells were purified from healthy donors' blood and expanded as explained in methods and material section. After day 15 of expansion primary NK cells were purified from the same donor (1×10⁶ cells/mL) and they were treated with IL-2 (1,000 U/mL), or the combination of IL-2 (1,000 U/mL) and anti-CD16mAb (3 μg/ml) for 18 hours. Expanded NK cells (1×10⁶ cells/mL) were reactivated with IL-2 (1,000 U/mL), and the combination of IL-2 (1,000 U/mL) and anti-CD16 mAb (3 μg/ml) for 18 hours. OSCSCs were labeled with ⁵¹Cr and the cytotoxicity of IL-2-treated and combination of IL-2 and anti-CD16mAb of primary (FIG. 8A) and expanded NK cells (FIG. 8B) against the OSCSCs was determined using the standard 4-hours ⁵¹Chromium release assay. Figure A and B is representative of one separate experiment. The fold decrease in cytotoxicity caused as the result of anti-CD16 mAb treatment was calculated (FIG. 8C).

FIG. 9A-FIG. 9F show that primary NK cells mediate higher level of ADCC than expanded NK cells. NK cells were purified from healthy donors' blood and expanded as explained in methods and material section. After day 15 of expansion primary NK cells were purified from the same donor (1×10⁶ cells/mL) and they left untreated, treated with IL-2 (1,000 U/mL), and the combination of IL-2 (1,000 U/mL) and anti-CD16mAb (3 μg/mL) for 18 hours. Expanded NK cells (1×10⁶ cells/mL) left untreated or reactivated with IL-2 (1,000 U/mL), and the combination of IL-2 (1,000 U/mL) and anti-CD16mAb (3 μg/mL) for 18 hours. OSCCS were labeled with ⁵¹Cr, and then left untreated or treated with anti-MICA/MICB antibody, or Cetaximab (5 μg/mL) for 30 minutes. The unbounded antibodies were washed and the cytotoxicity of Untreated (FIG. 9A). IL-2-treated (FIG. 9B), and combination of IL-2 and anti-CD16mAb (FIG. 9C) of primary and expanded NK cells against the OSCCs untreated or treated with the antibody against MICA/MICB antibody or Cetuximab was determined using the standard 4-hours Chromium-51 release assay. (FIGS. 9A-9C are reprehensive of one study). FIG. 9D shows the percentage of cytotoxicity for the same experiment. ADCC induced fold increase in IL-2 treated primary and expanded NK cells were calculated (FIG. 9E). Cytotoxicity of untreated, IL-2-treated (1,000 U/mL) primary NK, and day 15 IL-2-reactivated expanded NK (1,000 U/ml) form the same donor against the PL12 untreated or treated with the antibody against MICAS (5 μg/mL) was determined using the standard 4-hours Chromium-51 release assay (FIG. 9E).

FIG. 10A-FIG. 10D show that Mekabue extracted Fucoidan at a specific concentration induced split-anergy in NK cells, and A2 probiotic bacteria increased IFN-γ secretion in NK while their ability to mediate cytotoxicity remains unchanged. Mekabue were solubilized in PBS (12.5 mg/ml), Purified NK cells (1×10⁶ cells/ml) were treated with IL-2 (1000 U/ml) for 18 hours and the NK cells were cultured in absence or presence of Mekabue in 1:1000, and 1:100,000 titrations. The supernatant was harvested from the culture after 24 hours and the IFN-γ was measured by ELISA (FIG. 10A). Cells were resuspended in fresh media (1×10⁶ cells/ml). NK cells were then used as effector cells against ⁵¹Cr labeled OSCSCs. NK cell mediated cytotoxicity was determined using a standard 4-hour ⁵¹Cr release assay and the lytic units determined as described in methods and materials (FIG. 10B). NK cells were purified from peripheral blood and were treated with IL-2 (1000 U/mL) in the presence or absence of probiotic bacteria sAJ2 at 1:2 ratio (NK:sAJ2) for 18 hours. The supernatant was harvested from the culture after and the IFN-γ was measured by ELISA (FIG. 10C). NK cells were then used as effector cells against ⁵¹Cr labeled OSCSCs. NK cell mediated cytotoxicity was determined using a standard 4-hour ⁵¹Cr release assay and the lytic units were determined as described before (FIG. 10D).

FIG. 11A-FIG. 11B show that AJ2 probiotic bacteria and Mekabue can synergistically induce IFN-γ secretion in IL2-treated NK cells. Purified NK cells (1×10⁶ cells/ml) were treated with IL-2 (1000 U/ml) for 18 hours and the NK cells were cultured in absence or presence of Mekabue in 1:1000,000 titration, sAJ2 at 1:0.5 ratio (NK:sAJ2), or combination of sAJ2 and Mekabue. After 24 hours the supernatant was collected and IFN-γ was measured by ELISA. FIG. 11A is reprehensive of one experiment), FIG. 11B shows the scatter plot for three experiments.

FIG. 12A-FIG. 12B show the stage of differentiation in pancreatic tumors correlated with susceptibility to NK cell-mediated cytotoxicity. The surface expression of CD44, CD54, and MHC-class I on multiple pancreatic cell lines were assessed with flow cytometric analysis after staining with the respective PE-conjugated antibodies. Isotype control antibodies were used as control (FIG. 12A). Freshly isolated NK cells were left untreated or treated with anti-CD16mAb (3 μg/mL), IL-2 (1000 U/mL) or the combination of anti-CD16mAb (3 μg/ml) and IL-2 (1000 U/mL) for 18 h before they were added to ⁵¹Cr labeled MP2, Panc-1, BXPC3, HPAF, Capan and PL12. NK cell-mediated cytotoxicity was determined using a standard 4-hour ⁵¹Cr release assay and the lytic units 30/10⁶ cells were determined using inverse number of NK cells required to lyse 30% of the target cells×100 (FIG. 12B). One of the eight representative experiment is shown in the figure.

FIG. 13 shows lack of tumor growth, metastasis and long-term survival of NSG mice after orthotopic implantation of NK-supernatant differentiated MP2 tumors in pancreas MP2 tumors were differentiated by the NK-supernatants as described in the Materials and Methods section (diff-MP2). Patient-derived differentiated PL12 (2×10⁶) (n=3), NK-differentiated MP2 tumors (diff-MP2) (5×10⁵) (n=3), and MP2 tumors (3×10⁵) (n=3) were implanted into the pancreas of NSG mice. The rate of survival was assessed, and tumor growth in the pancreas and metastasis were determined after euthanasia.

FIG. 14A-FIG. 14B show that the combination of rhTNF-α and rhIFN-γ induce differentiation and resistance of MP2 cells to NK cell-mediated cytotoxicity. MP2 cells were left untreated or treated with rhTNF-α (20 ng/mL), rhIFN-γ (200 U/mL) or the combination of rhTNF-α (20 ng/mL) and rhIFN-γ (200 U/mL) for 24 h. Afterwards, the cells were detached and the surface expression of CD44, CD54, MHC-class I and B7H1 were assessed using PE conjugated antibodies followed by flow cytometric analysis. Isotype controls were used as controls (FIG. 14A). MP2 and Capan cells were treated as described in FIG. 14A, and were detached from the tissue culture plates, labeled with ⁵¹Cr and used in a standard 4-hour ⁵¹Cr release assay using IL-2 (1000 U/mL) treated NK cells. Pre-treatment of NK cells with IL-2 (1000 U/ml) were carried out for 18-24 hours. Percent cytotoxicity was determined at different effector to target ratio and the lytic units 30/10⁶ cells were determined using inverse number of NK cells required to lyse 30% of the tumor cells×100 (FIG. 14B). One of the eight representative experiment is shown in the figure.

FIG. 15A-FIG. 15G; Table S1; and Table S2 show the phenotypic characteristics of bone marrow, spleen, peripheral blood, pancreas and oral gingiva in hu-BLT mice. Hu-BLT mice were generated as depicted in FIG. 15A and described in Materials and Methods section, reconstitution of human immune system was analyzed in PBMCs, bone marrow and splenocytes using flow cytometric analysis after staining with anti-human and anti-mouse CD45 antibodies (n=6 per each experimental condition). The percentages of human CD45+ immune cells were determined within the total percentages of human and mouse CD45+ immune cells by staining with respective antibodies followed by flow cytometric analysis (FIG. 15B). PBMCs were isolated from hu-BLT mice and human donors as described in Materials and Methods section and percentages of CD3, CD16, CD56, CD19, and CD14 within human CD45+ immune-cells were determined using antibody staining followed by flow cytometric analysis (n=4 per each experimental condition) (FIG. 15C). PBMCs were isolated from hu-BLT mice, human donors and B6WT mice as described in Materials and Methods section and percentages of CD16, CD19, CD3, CD4, and CD8 within human CD45+ immune-cells were determined in human donor and hu-BLT mice PBMC and DX5, CD19. CD3, CD4 and CD8 with mouse CD45+ immune cells were determined in B6WT mice using antibody staining followed by flow cytometric analysis (n=4 per each experimental condition). The Table 1 shows the percentages of each subpopulations within CD45+ immune cells (FIG. 15D and Table 1). Single cell suspension from hu-BLT BM and spleen were isolated as described in Materials and Methods and percentages of CD16, CD56, CD3, CD33, CD14, and CD11b within human CD45+ immune-cells were determined using antibody staining followed by flow cytometric analysis (n=6 per each experimental condition) (FIG. 15E). Hu-BLT pancreas were harvested, single cells suspension was obtained as described in Materials and Methods and percentages of CD3, CD19, CD8, CD4, CD16, CD56 and CD14 within human CD45+ immune-cells in pancreas were determined using antibody staining followed by flow cytometric analysis (n=6 per each experimental condition) (FIG. 15F and Table 2). Oral gingiva from hu-BLT were harvested and single cell suspensions were prepared as described in Material and Methods section and were cultured in the presence of IL-2 (1000 U/ml) for 7 days. On day 7, the percentages of CD3, CD16 and CD56 within human CD45+ immune-cells were determined using antibody staining followed by flow cytometric analysis (n=6 per each experimental condition) (FIG. 15G).

FIG. 16A-FIG. 16D show that single injection of super-charged NK cells inhibited tumor growth and increased immune cells in the pancreas in hu-BLT mice. Hu-BLT mice were generated as described in Materials and Methods, and were implanted with 1×10⁶ tumor cells in the pancreas, and injected with 1.5×10⁶ super-charged NK cells via tail vein (FIG. 16A), and disease progression was monitored. After 6-7 weeks, mice were euthanized and pictures of the tumor within the pancreas were taken post-mortem (n=5 per each experimental condition) (FIG. 16B). Pancreatic cells were cultured with IL-2 (1×10⁶ cells/ml) for 3 days before the percentages of CD45+CD3 (FIG. 16C) and CD45+CD16 (FIG. 16D) were determined (n=3 per each experimental condition).

FIG. 17A-FIG. 17C show that single injection of super-charged NK cells inhibited tumor growth and increased immune cells in the pancreas in hu-BLT mice. Hu-BLT mice were generated as described in Materials and Methods section. Hu-BLT mice were implanted with 1×10⁶ tumor cells in the pancreas, and after 1-2 weeks, mice received 1.5×10⁶ super-charged NK cells via tail vein injection, and disease progression was monitored for another 3-5 weeks (n=6 per each experimental condition). MP2 tumors were differentiated by the NK-supernatants as described in the Materials and Methods section (diff-MP2). Hu-BLT mice were implanted with 1×10⁶ diff-MP2 tumor cells in the pancreas (FIG. 17A). The pancreas were harvested postmortem, and percentages of CD3 cells within human CD45+ immune cells from the pancreas were determined using antibody staining followed by flow cytometric analysis (n=3 per each experimental condition) (FIG. 17B). Pancreas were harvested after the mice were sacrificed followed by pancreatic cells isolation and, cells were cultured with IL-2 (1×10⁶ cells/ml) for 3 days before the percentages of CD45 were determined (n=3 per each experimental condition) (FIG. 17C).

FIG. 18A-FIG. 18L show that single injection of super-charged NK-cells with/without feeding with AJ2 inhibited tumor growth due to differentiation of tumors in hu-BLT mice. Hu-BLT mice were implanted with 1×10⁶ tumor cells in the pancreas, and after 1-2 weeks, mice received 1.5×10⁶ super-charged NK cells via tail vein injection, and disease progression was monitored for another 3-5 weeks. Mice were fed AJ2 (5 billion/dose) starting 1-2 weeks before tumor implantation, and thereafter every 48 hours throughout the experiment (n=20). At the end of experiment, mice were sacrificed, and pancreas/pancreatic tumor pictures were taken postmortem (FIG. 18A). Highly purified healthy human NK cells were treated with IL-2 (1000 U/mL) and anti-CD16mAb (3 μg/mL) for 18 hours, after which the supernatants were collected and added to MP2 tumors in the presence/absence of anti-TNF-α (1:100) and anti-IFN-γ (1:100) for a period of 5 days to induce NK-supernatant mediated differentiation. Hu-BLT mice were implanted with diff-MP2 (1×10⁶) or diff-MP2 treated with monoclonal antibodies against INF-γ and TNF-α (1×10⁶) and disease progression was monitored for another 3-5 weeks (n=6 per each experimental condition) (FIG. 18B). Hu-BLT mice were implanted with MP2 tumors and injected with NK cells as described in FIGS. 18A and 18B. At the end of the experiment pancreas/pancreatic-tumors were harvested and tumor growth was assessed on days 7, 11 and 14 (n=9-12 for each experimental condition); on day 7 attached tumors from each well were counted and equal numbers of tumors from each group were re-cultured and tumor growth in each well was determined every 3 days (FIGS. 18C and 18H). Hu-BLT mice were implanted with MP2 tumors and injected with NK cells as described in FIG. 18A, pancreas/pancreatic-tumors were resected and single-cell suspensions were prepared, as described in the Materials and Methods section, and they were cultured for 7 days before the pictures of the culture were taken (FIG. 18D). Hu-BLT mice were orthotopically injected with 1×10⁶ of undifferentiated MP2 or NK-differentiated-MP2 cells as described in FIGS. 17A and 18B. One or two weeks after the undifferentiated MP2 tumor implantation selected hu-BLT mice received 1.5×10⁶ super-charged NK cells via tail vein injection. At the end of this experiment, pancreas/pancreatic tumors were harvested and single cells suspensions were prepared as described in Materials and Methods. Pancreas/pancreatic tumor cells from the mice were cultured, the pictures of culture were taken on day 7. One of the four representative experiment is shown in FIG. 18E. Hu-BLT mice were implanted with MP2 tumors and injected with NK cells and fed with AJ2 as described in FIG. 18A. At the end of the experiment pancreas/pancreatic-tumors were harvested and tumor growth was assessed on days 7, 11 and 14 (n=12 for each experimental condition); on day 7 attached tumors from each well were counted and equal numbers of tumors from each group were re-cultured and tumor growth in each well was determined every 3 days (FIGS. 18F and 18I). Procedures were carried out as described in Fig. S5A using injections of allogeneic or autologous super-charged NK cells. Pancreatic tumors were resected and single-cell suspensions were prepared and tumor growth was assessed on days 7, 11 and 14 (n=9 to 12 for each experimental condition) using identical numbers of tumors cultured from each mouse tumor (FIG. 18G). Hu-BLT mice were implanted with tumors and injected with super-charged NK cells, as described in FIG. 18A. Tumors were resected, and single cell cultures were prepared and cultured for 7 days, after which percentages of human CD45, CD94, CD56, NKG2D, and DNAM within the tumors were determined after staining with antibodies, followed by flow cytometric analysis (n=4 for each experimental condition) (FIGS. 18J and 18K). Hu-BLT mice were implanted with MP2 tumors and injected with NK cells and fed with AJ2 as described in FIG. 18A. At the end of the experiment, tumors were resected, and single cell cultures were prepared, as described in FIG. 18C, and equal numbers of tumors were cultured on day 7 and 11 and the levels of IFN-γ were determined in culture supernatants (n=2 for each experimental condition) (FIG. 18L).

FIG. 19A-FIG. 19J show that single injection of super-charged NK-cells with/without feeding with AJ2 inhibited tumor growth due to differentiation of tumors in hu-BLT mice.

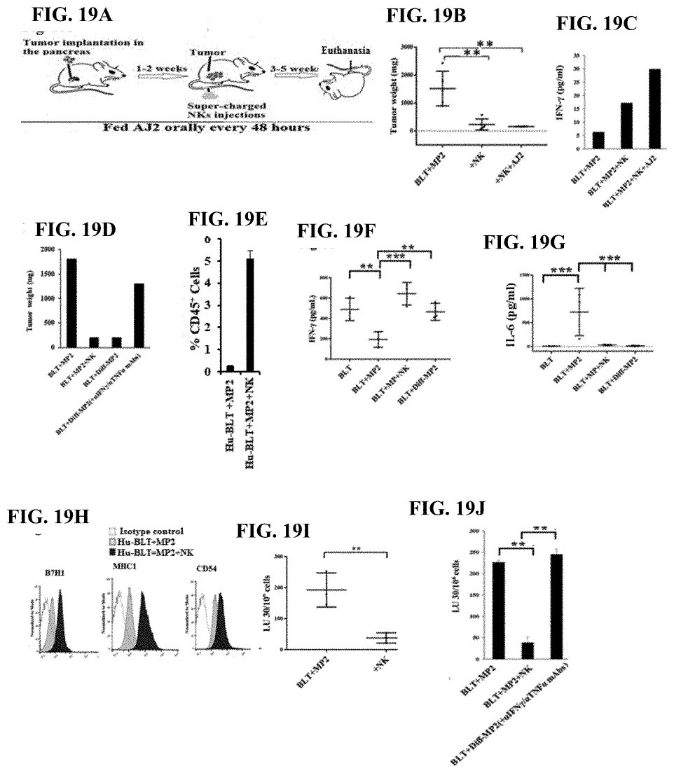

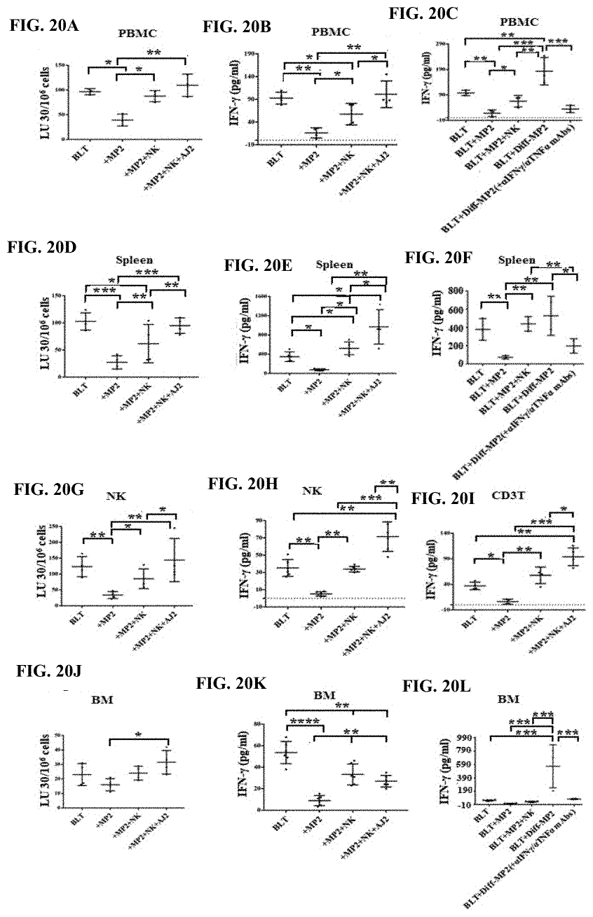

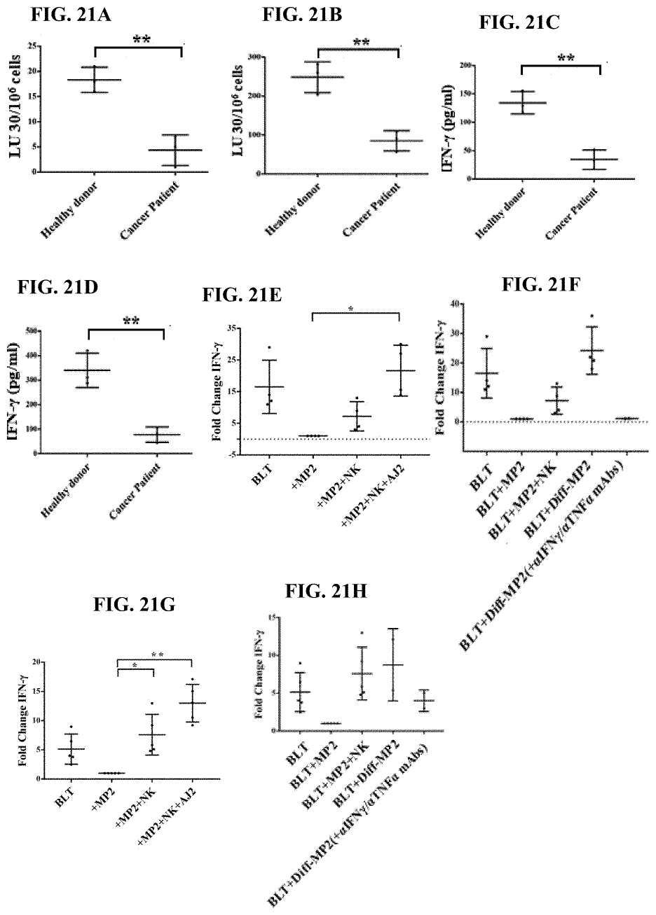

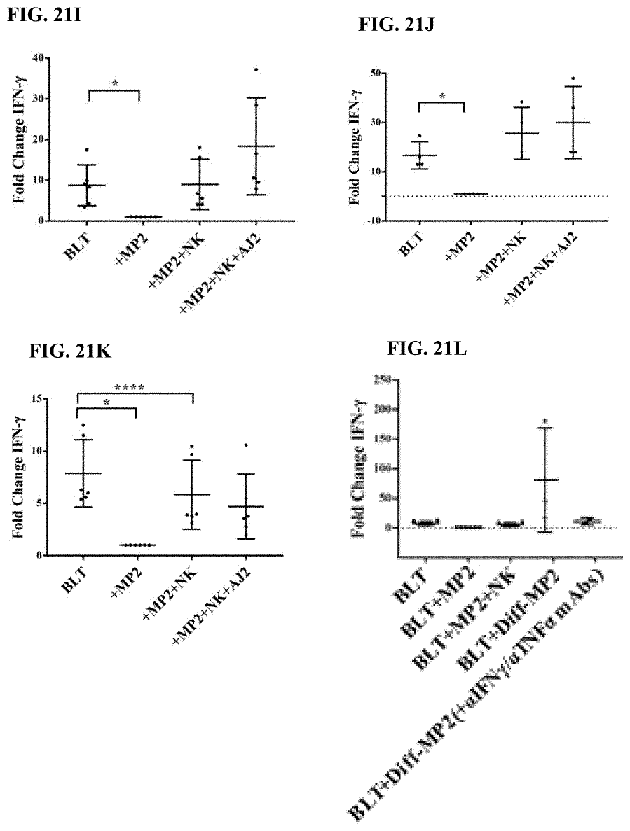

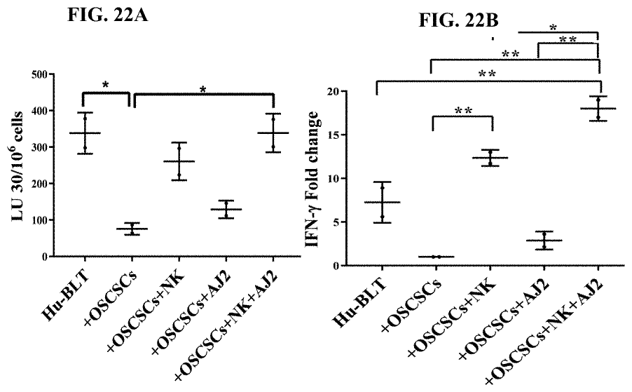

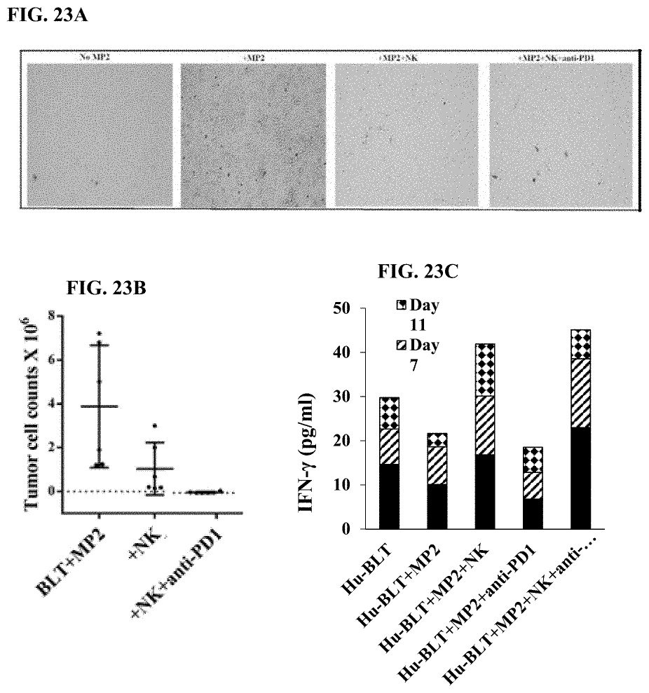

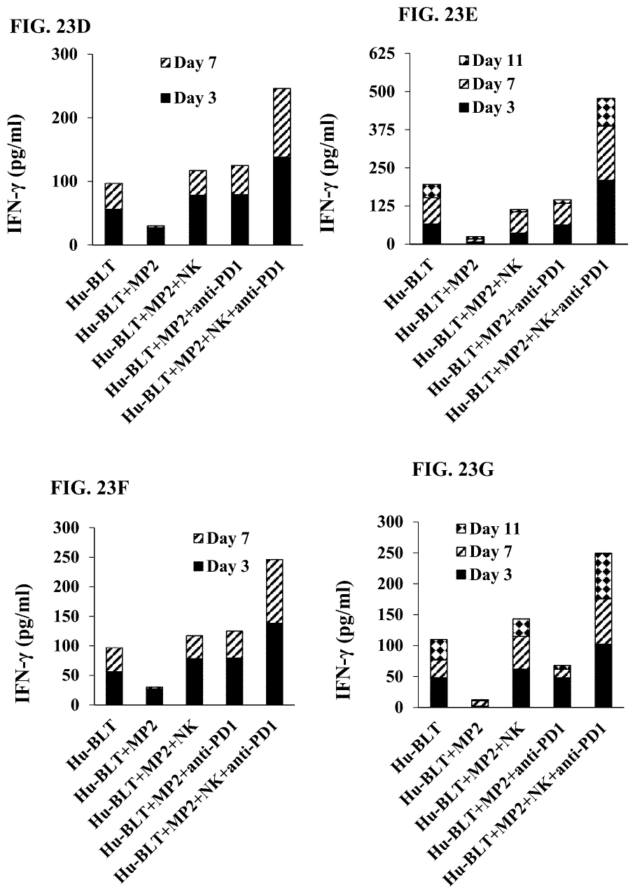

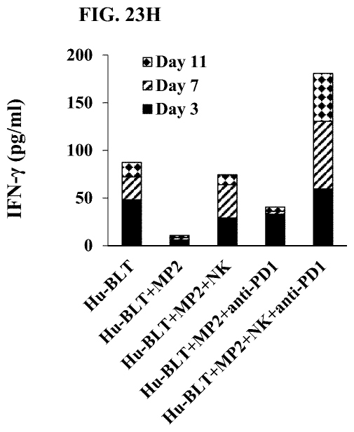

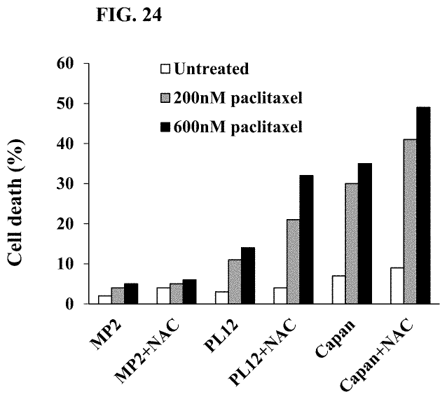

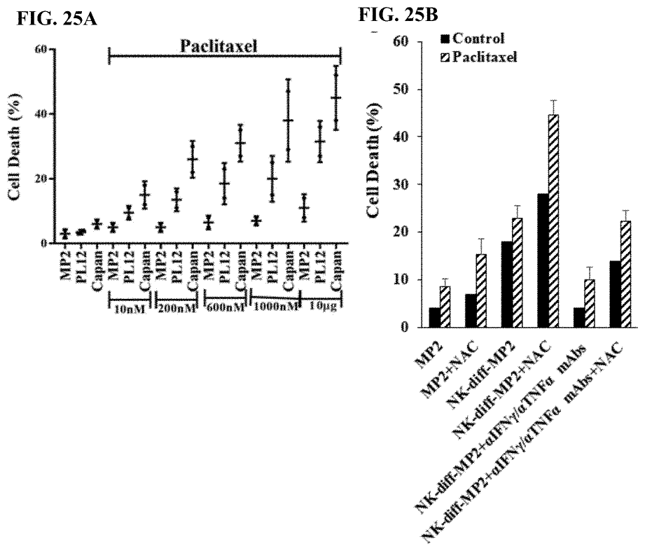

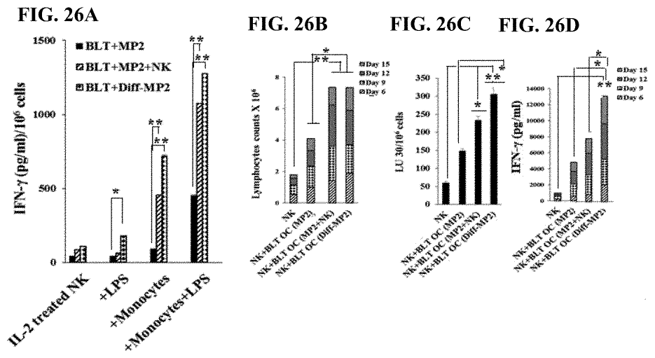

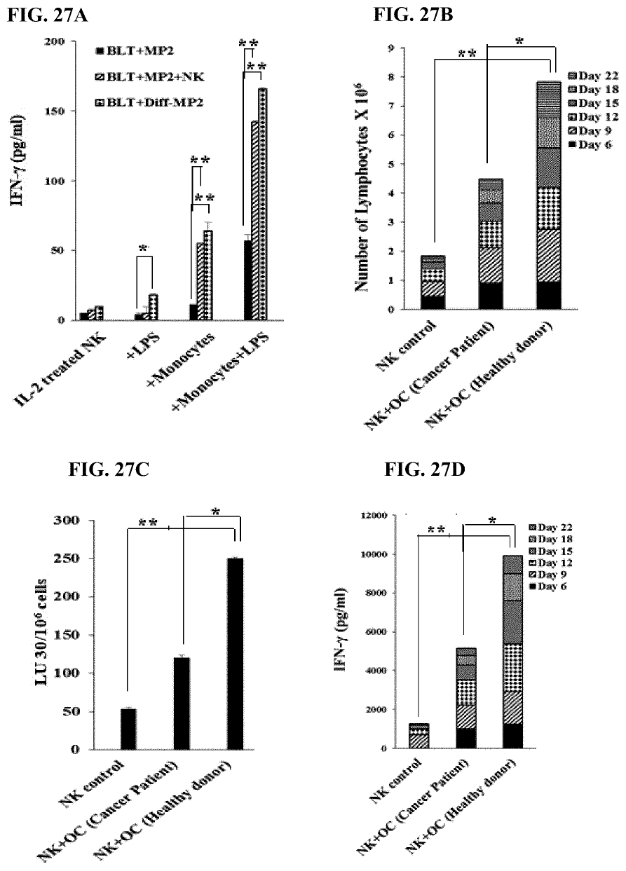

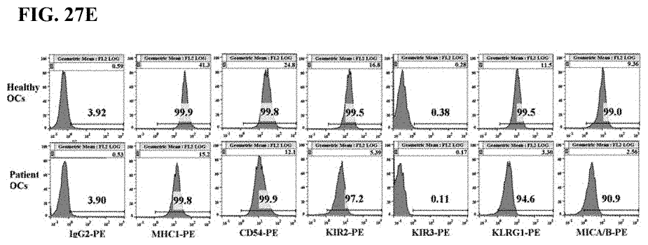

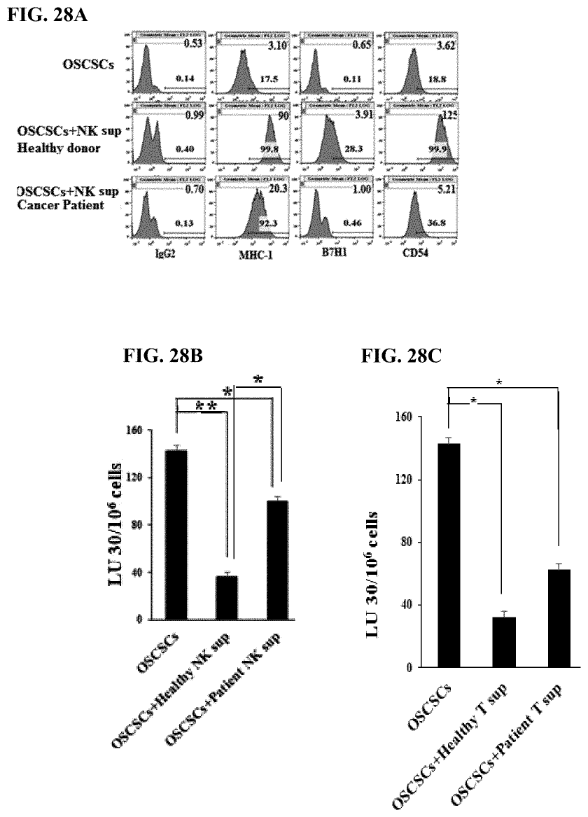

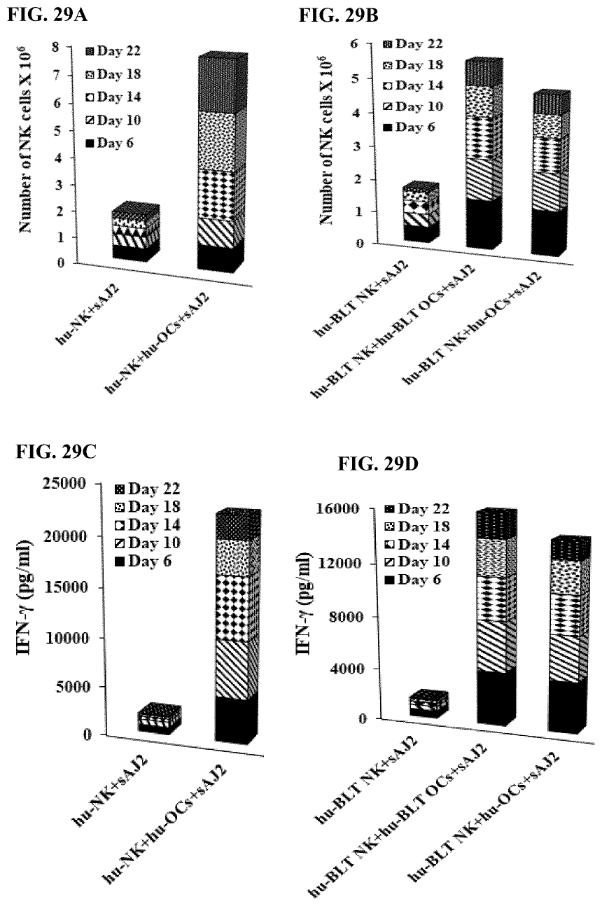

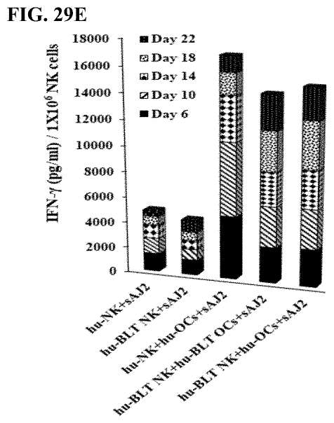

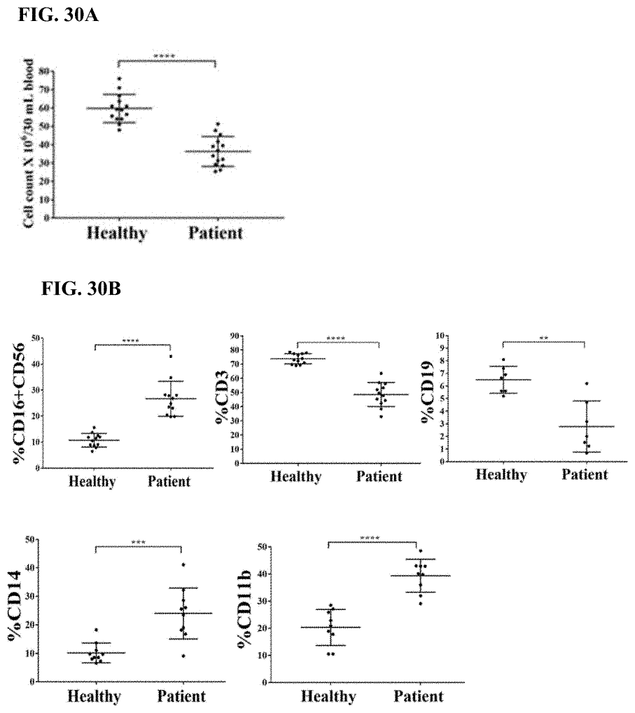

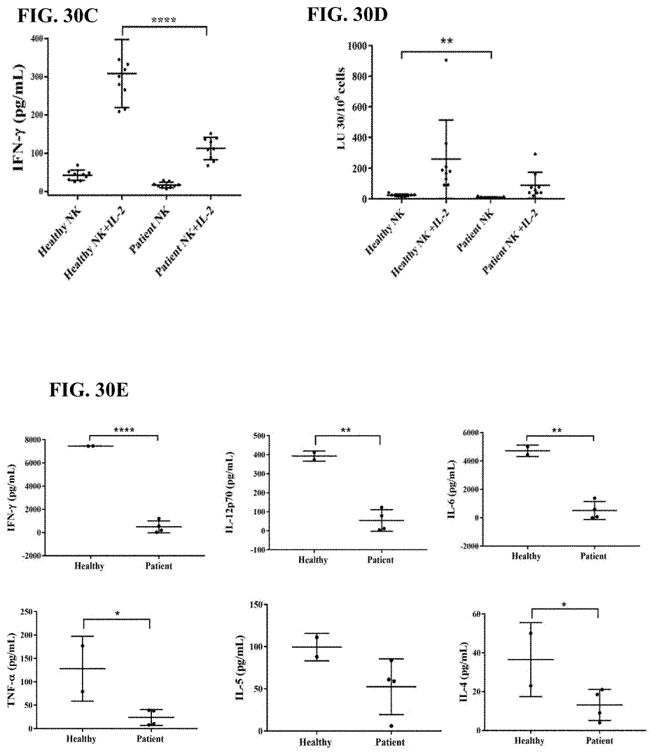

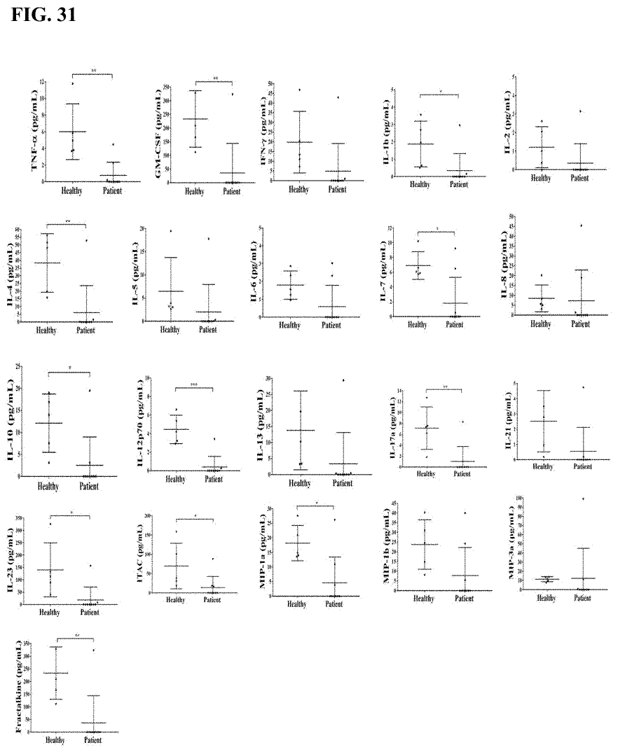

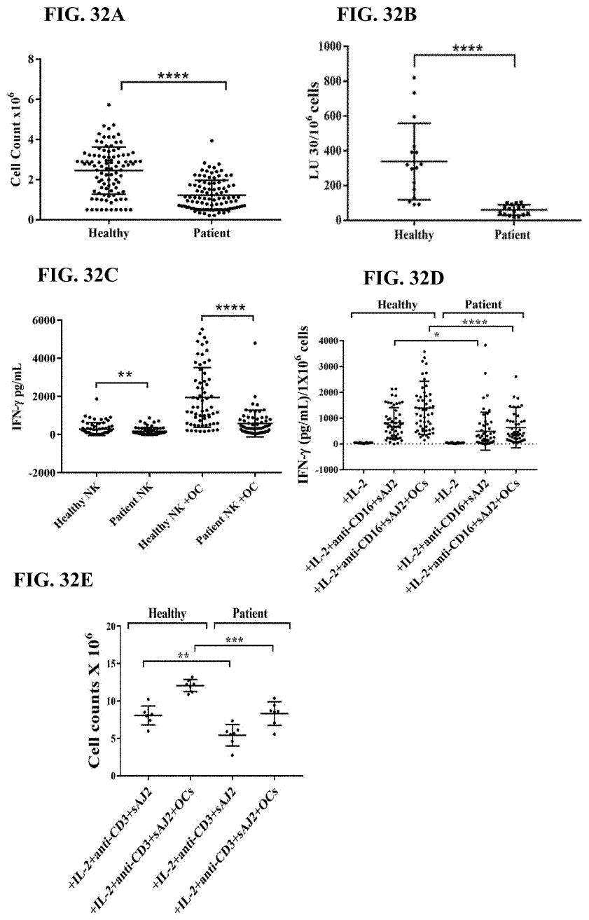

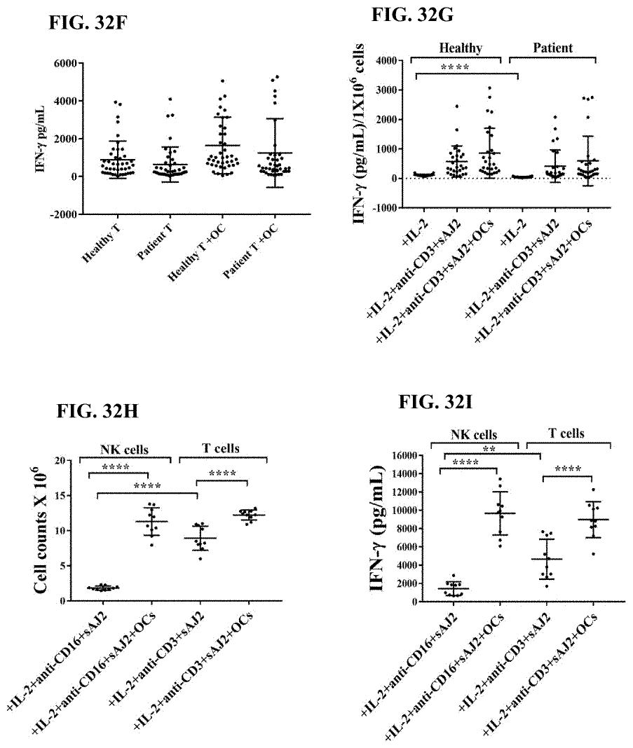

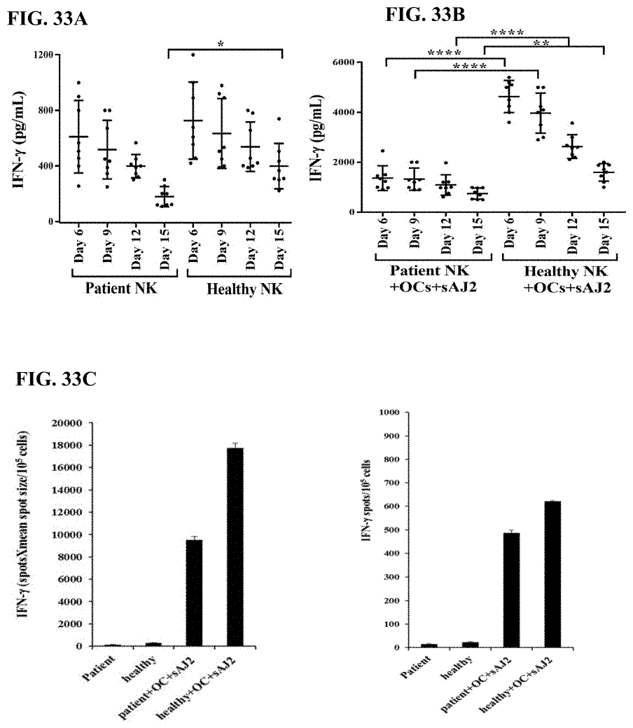

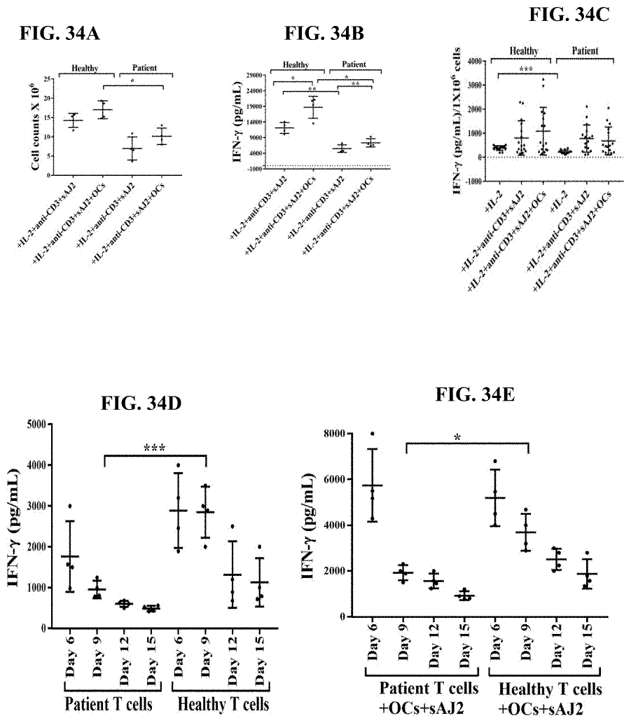

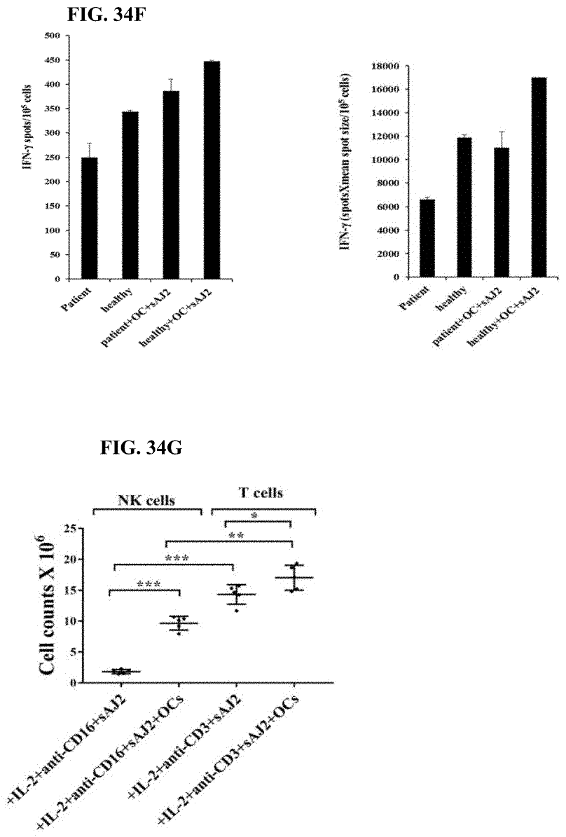

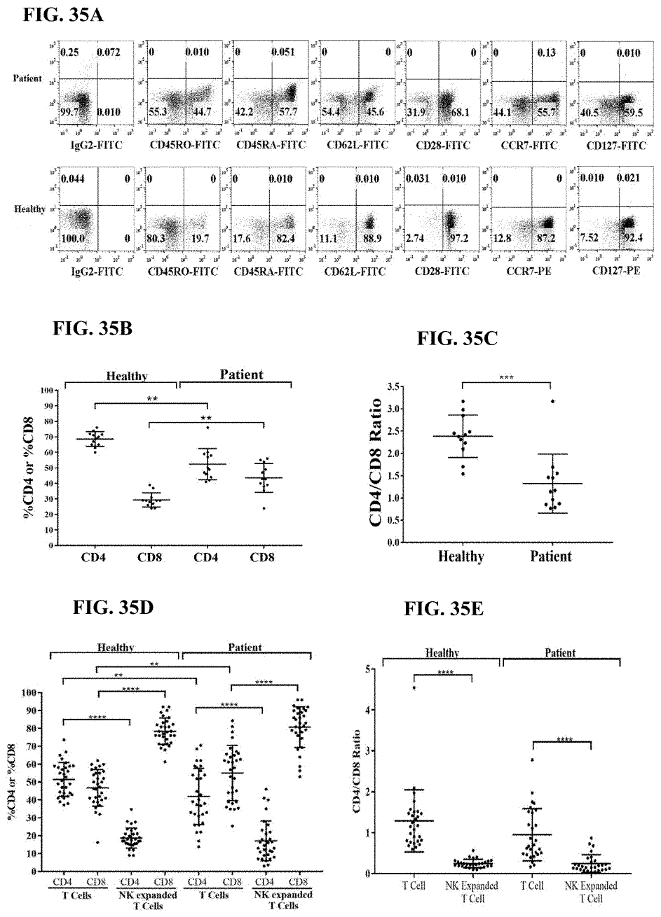

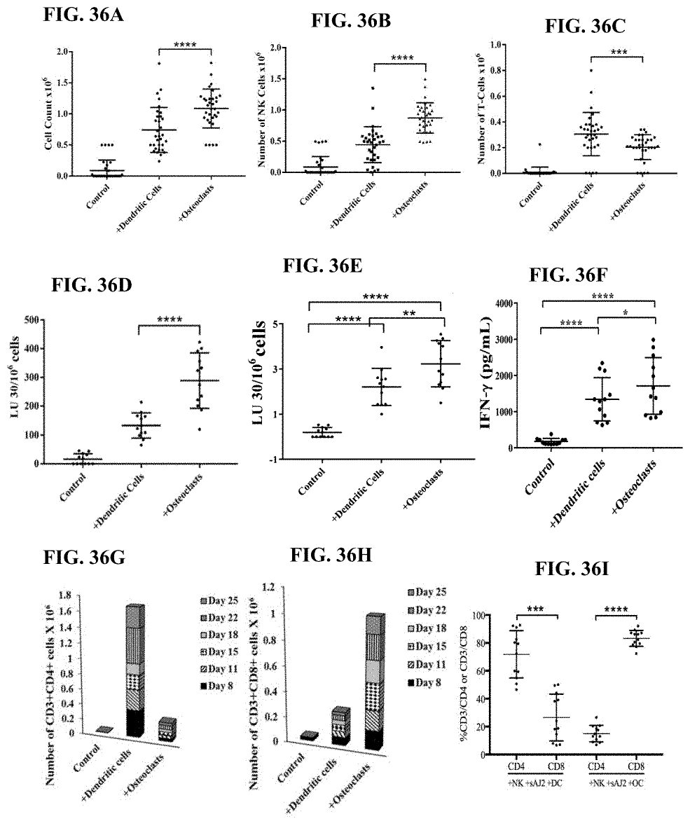

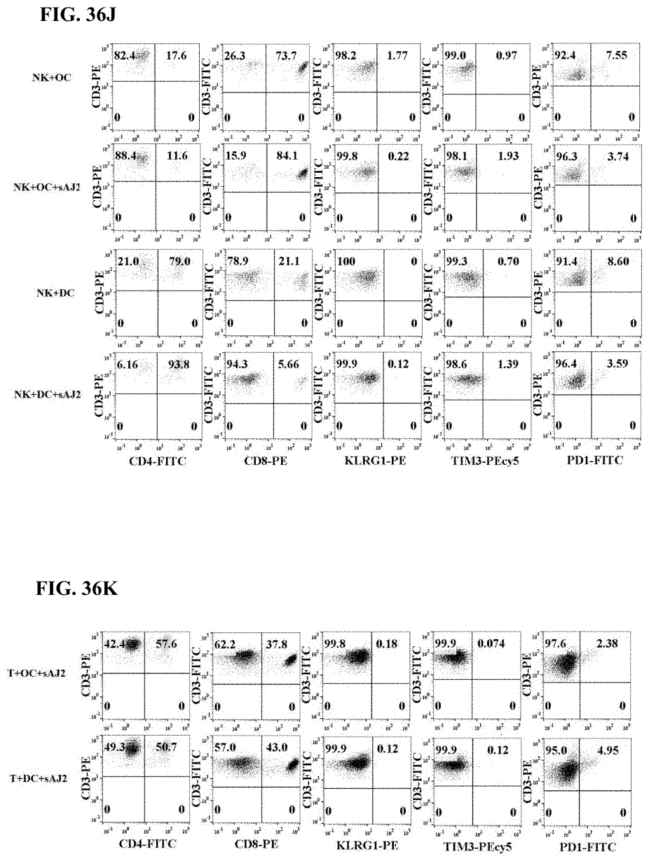

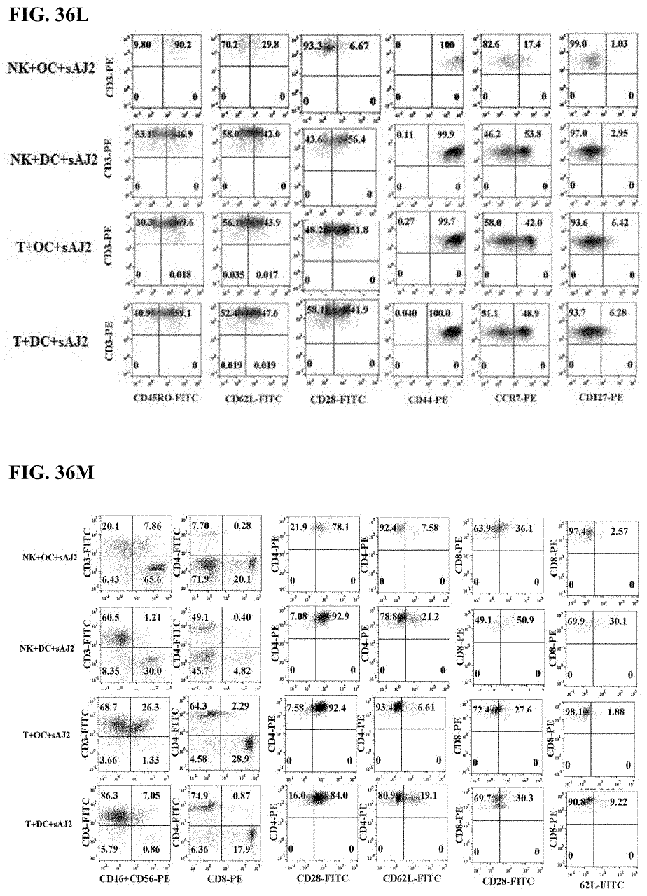

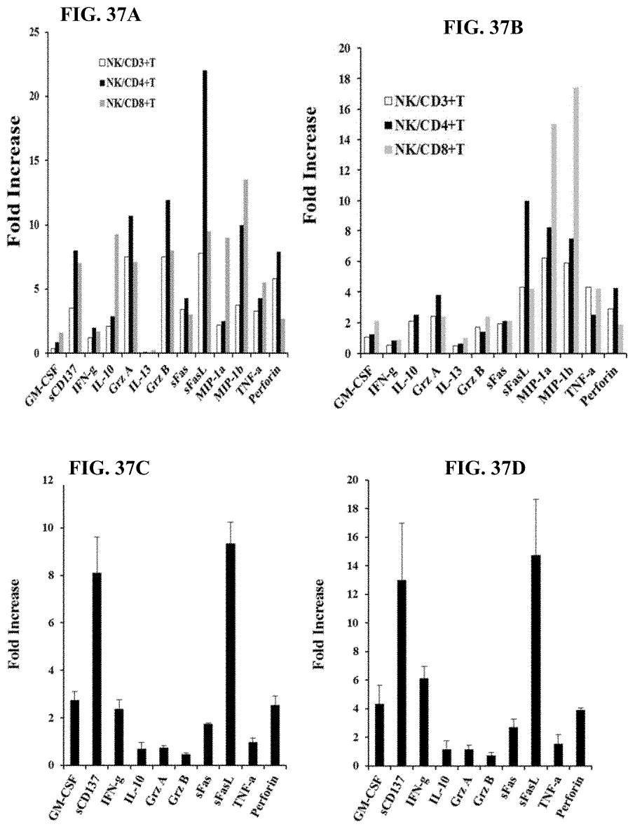

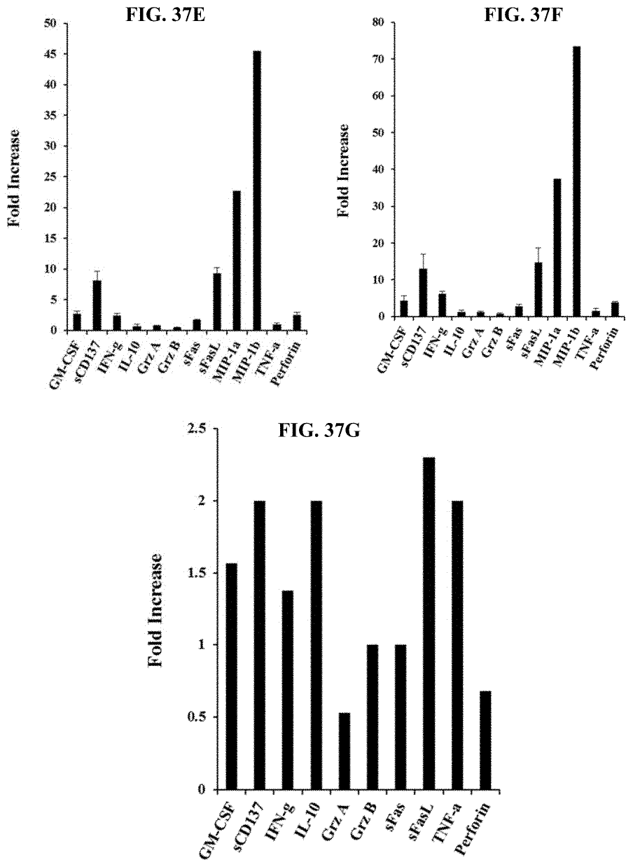

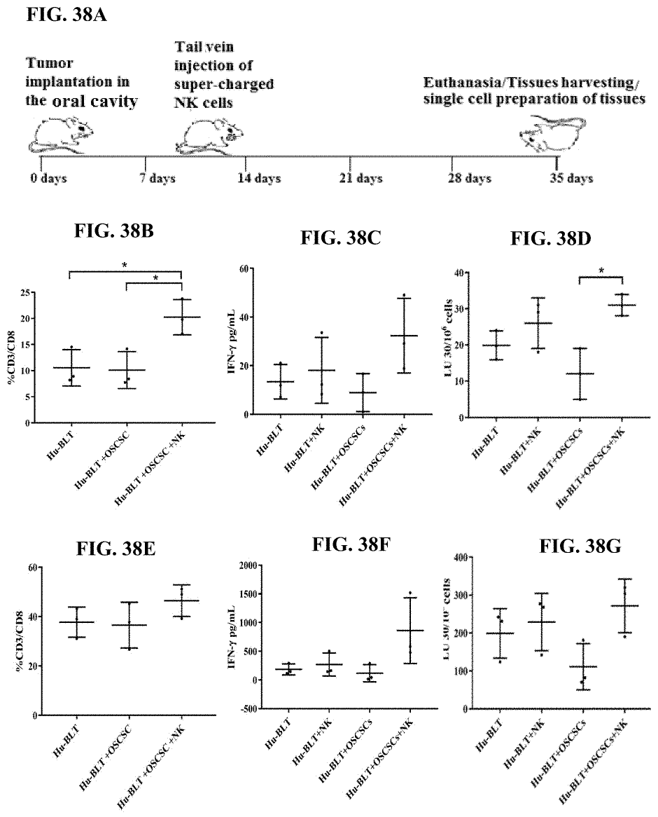

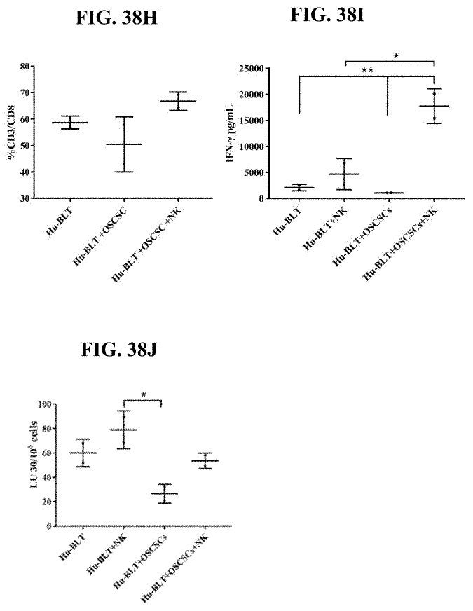

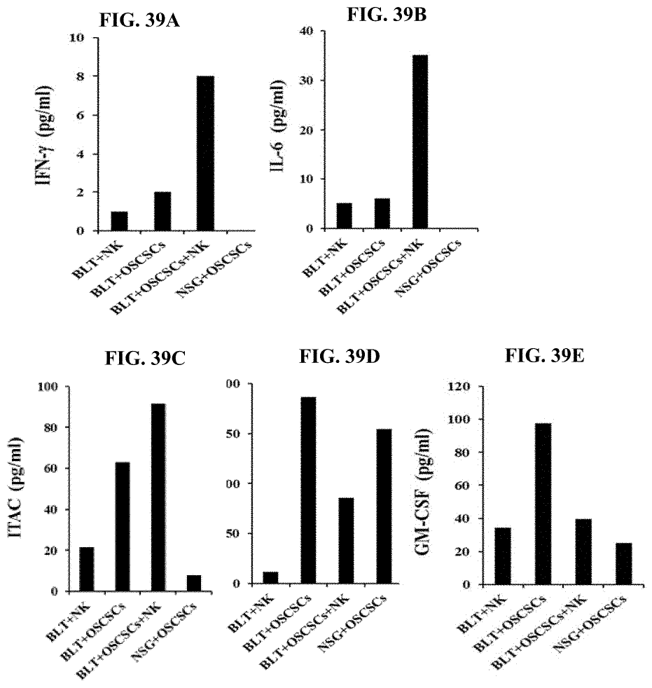

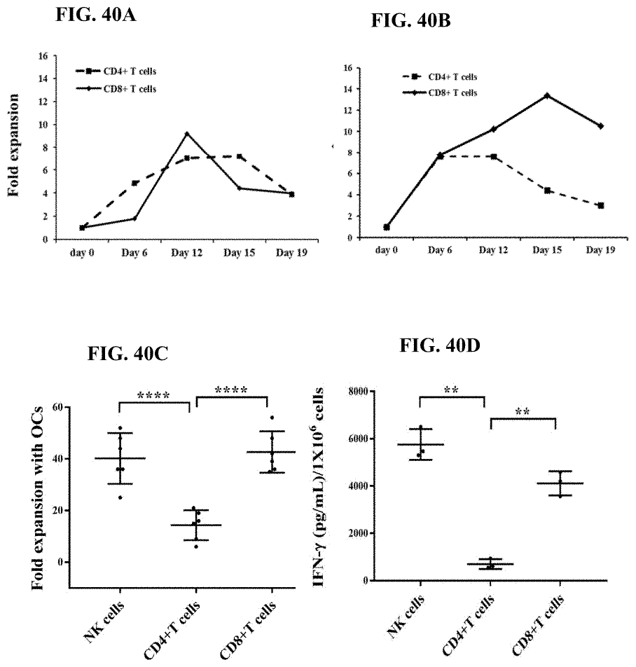

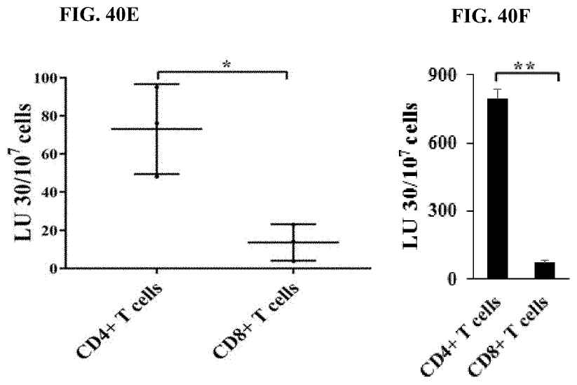

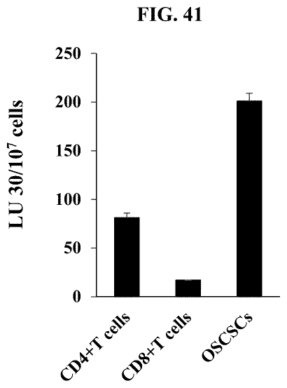

Procedures were carried out as depicted in this figure, and disease progression was monitored (FIG. 19A). Mice were sacrificed, and pancreatic tumors were harvested and weighed (n=4 per each experimental condition) (FIG. 19B). Procedures were carried out as described in FIG. 19A and multiplex array was used to determine IFN-γ in the peripheral-blood derived serum of mice. One of three representative experiments is shown (FIG. 19C). Procedures were carried out as described in FIG. 19A before pancreatic tumors were harvested and weighed. One of three representative experiments is shown (FIG. 19D). Procedures were carried out as described in FIG. 19A. Pancreas were removed, and single cell cultures were prepared after which percentages of human CD45+ immune cells were determined (n=4 per each experimental condition) (FIG. 19E). Procedures were carried out as described in FIG. 19A. Pancreas were removed, and single cell cultures were prepared and equal numbers of pancreatic cells were cultured until day 7 and 11 and the levels of IFN-γ (FIG. 19F) and IL-6 (FIG. 19G) were determined in culture supernatants (n=4 per each experimental condition). Procedures were carried out as described in FIG. 19A. Tumors were resected, and single cell cultures were prepared and surface expressions of MHC-class I, B7H1 and CD54 were determined on tumors after 11 days of culture (n=4) (FIG. 19H). NK cells (1×10⁶ cells/mL) from healthy human donors were treated with IL-2 (1000 U/mL) for 18 hours before they were added to ⁵¹Cr labeled tumors obtained from mice implanted with different tumors and/or injected with NK cells as described in FIG. 19A at various effector to target ratios. NK-mediated cytotoxicity was determined using 4-hour ⁵¹Cr release assay. The lytic units (LUs) 30/10⁶ cells were determined using inverse number of NK cells required to lyse 30% of the tumor-cells×100 (n=3) (FIGS. 19I and 19J).

FIG. 20A-FIG. 20L show that injection of super-charged NK-cells with/without feeding with AJ2 restored and increased IFN-γ secretion and/or cytotoxic function of NK cells from different tissues of tumor-bearing hu-BLT mice. Procedures were carried out as described in FIG. 19A. Upon sacrifice, PBMCs were isolated from blood and treated with IL-2 (1000 U/mL) before they were used in cytotoxicity assay against OSCSCs using 4-hour ⁵¹Cr release assay. LUs 30/10⁶ were determined, as described in FIG. 19I (n=3 per each experimental condition) (FIG. 20A). Procedures were carried out as described in FIG. 19A before the PBMCs were isolated and treated with (1000 U/mL) and the supernatants were harvested and IFN-γ secretion was determined using ELISA (n=4 per each experimental condition) (FIGS. 20B and 20C). Procedures were carried out as described in FIG. 19A before spleens were harvested, and single-cell suspensions were prepared. Splenocytes were treated with IL-2 (1000 U/mL) before they were used for cytotoxicity against OSCSCs using 4-hour ⁵¹Cr release assay. LU 30/10⁶ were determined, as described in FIG. 19I (n=4 per each experimental condition) (FIG. 20D). The supernatants were harvested from day 3 and 7 cultures of splenocytes, and IFN-γ secretion was determined using ELISA (n=5 per each experimental condition) (FIGS. 20E and 20F). NK-enriched cells were isolated from splenocytes and were cultured with IL-2 (1000 U/mL) before they were used for cytotoxicity against OSCSCs using 4-hour ⁵¹Cr release assay. LUs 30/10⁶ were determined, as described in FIG. 19I (n=4 per each experimental condition) (FIG. 20G). Supernatants were harvested from day 3 and 7 NK-enriched cultures and IFN-γ secretion was determined using single ELISA (n=6 per each experimental condition) (FIG. 20H). The CD3T-cells were isolated from splenocytes and were cultured with IL-2 (100 U/mL), and on day 3 and day 7 the supernatants were harvested and IFN-γ secretion was determined using ELISA (n=4 per each experimental condition) (FIG. 20I). Procedures were carried out as described in FIG. 19A. BM cells were harvested and treated with IL-2 (1000 U/mL) for 7 days before they were used for cytotoxicity against OSCSCs using 4-hour ⁵¹Cr release assay. LUs 30/10⁶ were determined, as described in FIG. 19I (n=4 per each experimental condition) (FIG. 20J). Supernatants were harvested on day 3 and day 7 of BM culture and IFN-γ secretion was determined using ELISA (n=6 per each experimental condition) (FIGS. 20K and 20L).

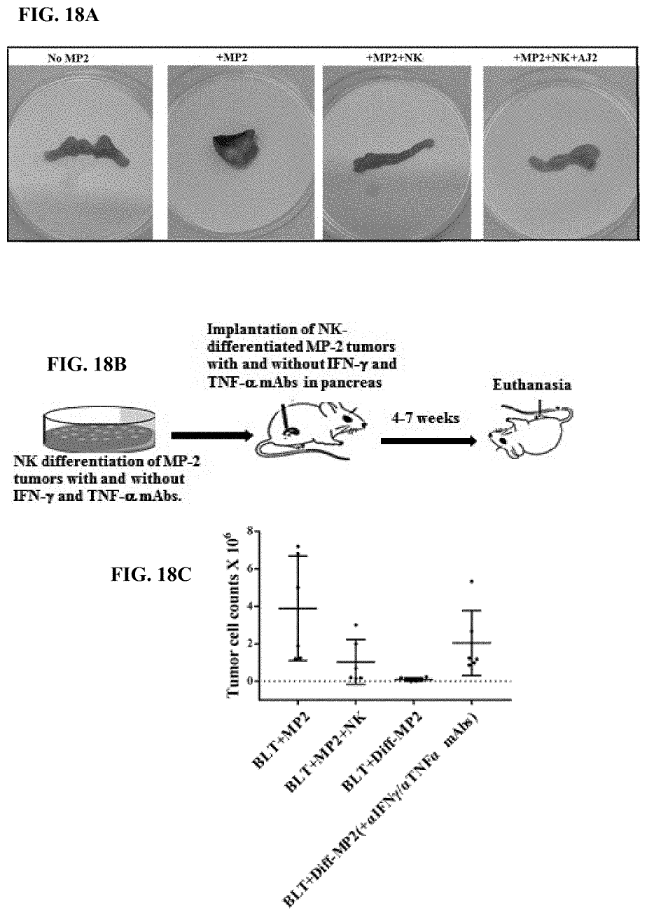

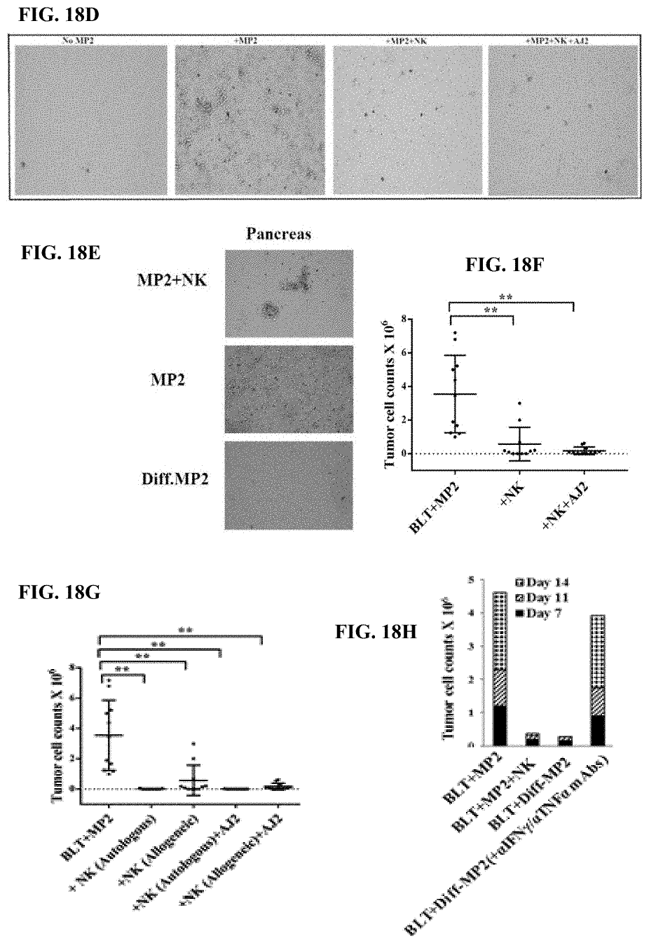

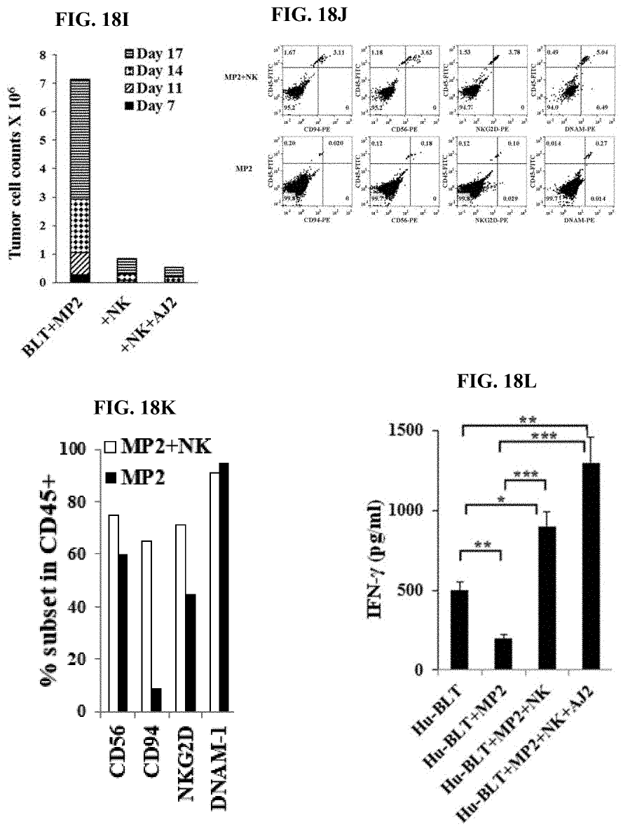

FIG. 21A-FIG. 21L show that injection of super-charged NK-cells with/without feeding with AJ2 restored and increased IFN-γ secretion in peripheral blood, spleen, BM, enriched-NK cells and purified CD3+ T-cells in tumor-bearing hu-BLT mice. PBMCs (FIG. 21A) and purified NK cells (FIG. 21B) from healthy human donors and pancreatic cancer patients were obtained and treated with IL-2 (1000 U/mL) for 18 hours before they were used for cytotoxicity against OSCSCs using 4-hour ⁵¹Cr release assay. The lytic units (LUs) 30/10⁶ cells were determined using inverse number of NK cells required to lyse 30% of the tumor-cells×100 (n=3 for each experimental condition) (FIGS. 21A and 21B). PBMCs (FIG. 21C) and purified NK cells (FIG. 21D) from healthy human donors and pancreatic cancer patients were treated with IL-2 (1000 U/mL) for 18 hours before the supernatants were harvested and IFN-γ secretion was determined using ELISA (n=4 for each experimental condition) (FIGS. 21C and 21D). Hu-BLT mice were implanted with tumors and injected with NK cells, and were fed with/without AJ2, as described in FIG. 18A. At the end of the experiment, PBMCs were isolated and treated with IL-2 (1000 U/mL) for 7 days before the supernatants were harvested and IFN-γ secretion was determined using single ELISA and the fold changes of IFN-γ release were determined using secreted amount of IFN-γ from PBMCs of tumor-alone injected hu-BLT as the base (n=4 for each experimental condition) (FIG. 21E). Hu-BLT mice were implanted with MP2 tumors and injected with NK cells as described in FIG. 18A and implanted with NK-differentiated MP2 and diff-MP2 treated with monoclonal antibodies against INF-γ and TNF-α as described in FIG. 18B. After euthanasia PBMCs were isolated and treated with (1000 U/mL) for 7 days before the supernatants were harvested and IFN-γ secretion was determined using single ELISA, and the fold changes of IFN-γ release were determined using secreted amount of IFN-γ from PBMCs of tumor-alone injected hu-BLT as the base (n=4 for each experimental condition) (FIG. 21F). Hu-BLT mice were implanted with tumors and injected with NK cells, and were fed with/without AJ2, as described in FIG. 18A. At the end of the experiment, spleens were harvested and splenocytes were isolated and treated with IL-2 (1000 U/mL) for 7 days before the supernatants were harvested and IFN-γ secretion was determined using single ELISA and the fold changes of IFN-γ release were determined using secreted amount of IFN-γ from splenocytes of tumor-alone injected hu-BLT as the base (n=4 for each experimental condition) (FIG. 21G). Hu-BLT mice were implanted with MP2 tumors and injected with NK cells as described in FIG. 18A and implanted with NK-differentiated MP2 and diff-MP2 treated with monoclonal antibodies against INF-γ and TNF-α as described in FIG. 18B. After euthanasia spleen were harvested and splenocytes were isolated and treated with (1000 U/mL) for 7 days before the supernatants were harvested and IFN-γ secretion was determined using single ELISA, and the fold changes of IFN-γ release were determined using secreted amount of IFN-γ from splenocytes of tumor-alone injected hu-BLT as the base (n=4 for each experimental condition) (FIG. 21H). NK-enriched cells were isolated from splenocytes, as described in Materials and Methods, and were cultured with IL-2 (1000 U/mL) for 7 days before the supernatants were harvested from day 3 and 7 NK-enriched cultures and IFN-γ secretion was determined using single ELISA and fold changes in the IFN-γ release were determined using the secreted amount of IFN-γ from NK-enriched cells of tumor-alone injected hu-BLT as the base (n=6 for each experimental condition) (FIG. 21I). The CD3T-cells were isolated from splenocytes, as described in Materials and Methods, and were cultured with IL-2 (100 U/mL), and on day 3 and day 7 the supernatants were harvested and IFN-γ secretion was determined using single ELISA and fold changes in IFN-γ release were determined using secreted amount of IFN-γ from CD3T-cells from tumor-alone injected hu-BLT as base (n=4 for each experimental condition) (FIG. 21J). Hu-BLT mice were implanted with tumors and injected with NK cells, and were fed with/without AJ2, as described in FIG. 18A. At the end of the experiment, bone marrow cells were isolated and treated with IL-2 (1000 U/mL) for 7 days before the supernatants were harvested and IFN-γ secretion was determined using single ELISA and the fold changes of IFN-γ release were determined using secreted amount of IFN-γ from bone marrow cells of tumor-alone injected hu-BLT as the base (n=4 for each experimental condition) (FIG. 21K). Hu-BLT mice were implanted with MP2 tumors and injected with NK cells as described in FIG. 18A and implanted with NK-differentiated MP2 and diff-MP2 treated with monoclonal antibodies against INF-γ and TNF-α as described in FIG. 18B. After euthanasia bone marrow cells were isolated and treated with (1000 U/mL) for 7 days before the supernatants were harvested and IFN-γ secretion was determined using single ELISA, and the fold changes of IFN-γ release were determined using secreted amount of IFN-γ from bone marrow cells of tumor-alone injected hu-BLT as the base (n=4 for each experimental condition) (FIG. 21L).

FIG. 22A-FIG. 22B show that injection of super-charged NK cells with/without feeding AJ2 restored and increased IFN-γ secretion and cytotoxic function of NK-cells in the peripheral blood of oral tumor injected hu-BLT mice. Hu-BLT mice were implanted with human OSCSCs and injected with NK cells, and fed AJ2 as described in Materials and Methods. Following sacrifice peripheral blood was collected, PBMCs were isolated were treated with IL-2 (1000 U/ml) for 7 days. On day 7, cytotoxicity assay was performed using standard 4-hour ⁵¹Cr release assay against OSCSCs, and the LU 30/10⁶ cells were determined using inverse number of cells required to lyse 30% of OSCSCs×100 (FIG. 22A). The supernatants were harvested and the level of IFN-γ was determined using specific ELISA. Fold changes in IFN-γ secretion in each tissue from each group of mice were determined over those obtained from mice injected with OSCSCs alone (FIG. 22B).

FIG. 23A-FIG. 2311 show that combination of super-charged NK cells with anti-PD1 antibody injection increased IFN-γ secretion substantially by PBMCs, splenocytes and bone marrow derived immune cells and halted growth of poorly differentiated MP2 tumors in hu-BLT mice. Successfully reconstituted hu-BLT mice were orthotopically injected with 1×10⁶ of human MP2 cells in the pancreas. One or two weeks after tumor implantation selected hu-BLT mice received 1.5×10⁶ super-charged NK cells via tail vein injection. Seven days later, anti-PD1 (50 μg/mice) was injected via tail vain injection. At the end of experiment, animals were sacrificed and pancreas were harvested and the single cells suspensions were prepared from the mice's pancreas as described in Materials and Methods, and were cultured for 7 days when the pictures of culture were taken (FIG. 23A). The freshly dissected pancreatic tumors was dissociated to recover single cells, cells were counted during the culture on day 7, 11, 14 and 18 (FIG. 23B). Pancreatic cells from the mice were cultured, on day 3, 7 and 11, while the supernatants were harvested from cultures and IFN-γ secretion was determined using single ELISA (FIG. 23C). Peripheral blood (FIG. 23D), spleen (FIG. 23E), bone marrow (FIG. 23F) were harvested from hu-BLT mice and single cell suspensions were obtained as described in Materials and Methods. Cells were treated with IL-2 (1000 U/ml), on days as specified in the figures and supernatants were harvested from cultures and IFN-γ secretion was determined using single ELISA (FIGS. 23D-23F). NK enriched cells (FIG. 23G) and CD3+ T cells (FIG. 23H) isolated from hu-BLT splenocytes were treated with IL-2 (1000 U/ml and 100 U/ml respectively), on days as specified in the figures and supernatants were harvested from cultured and IFN-γ secretion was determined using single ELISA (FIGS. 23G-23H). One of the two representative experiment is shown in the figure.

FIG. 24 shows increased induction of cell death by NAC in well differentiated PL12 and Capan and less in poorly differentiated stem-like MP2; effect on Paclitaxel mediated cell death.

MP2, PL12 and Capan cells were treated with or without NAC (20 nM) for 24 hours, followed by treatment with Paclitaxel (200 and 600 nM) for 18-24 hours. All samples start with the same number of cells (100,000 per well in a 24 well plate) treated with different treatment modalities. Afterwards, the viability of untreated and treated MP2, PL12 and Capan cells were determined using propidium iodide and analyzed with flow cytometry. One of the five representative experiment is shown in the figure.

FIG. 25A-FIG. 25B show that paclitaxel induced significant cell death in patient-derived differentiated PL12 and Capan and NK-differentiated MP2 tumors treated with and without NAC

MP2, PL12, and Capan tumors (1×10⁵ tumors/well) were treated with or without Paclitaxel for 18-20 hours before the viability of cells was determined using propidium iodide staining. P-values of <0.05 were obtained for differences between MP2 vs. PL12 and Capan at the concentrations of 10 nM, 200 nM, 600 nM, 1000 nM and 10 μg of Paclitaxel (n=2 per each experimental condition) (FIG. 25A). MP2 tumors were differentiated with NK supernatants in the presence and absence of anti-IFN-γ and anti-TNF-α as described in Materials and Methods for a period of 5 days before they were washed and treated with/without NAC (20 nM) for 24 hours, followed by paclitaxel treatment at 10 nM-600 nM range for 18-24 hours. The viability of cells was determined by staining with propidium iodide (n=3 per each experimental condition) (FIG. 25B).

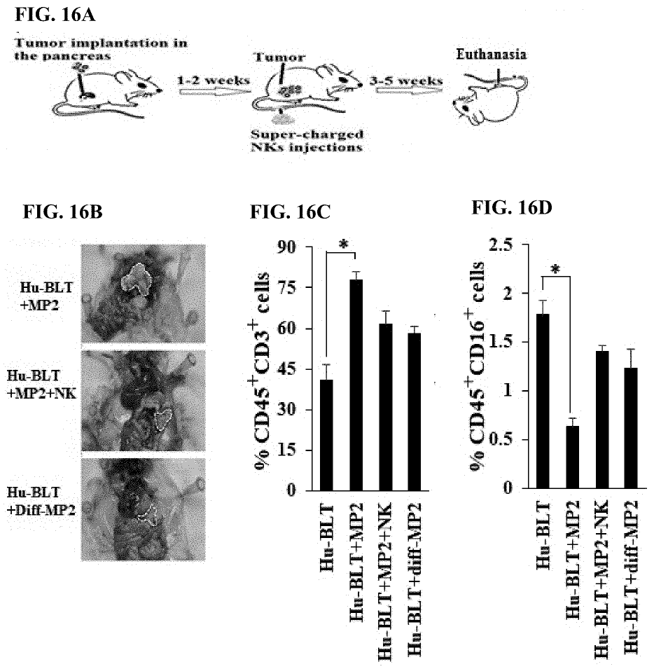

FIG. 26A-FIG. 26D show that monocytes or osteoclasts from tumor-bearing mice injected with NK cells or implanted with only NK-differentiated MP2 tumors triggered significantly more IFN-γ from NK cells when compared to those of tumor-alone implanted mice Hu-BLT mice were implanted with tumors and injected with NK cells, as described in FIG. 16A before spleen and BM were harvested and single cell suspensions were prepared after mice were euthanized. CD56+NK cells were positively selected from splenocytes, and monocytes were purified from BM cells, and co-cultured at (NK:Monocytes; 2:1 ratio) and treated with IL-2 (1000U/ml) alone or in combination with LPS (100 ng/mL) for 7 days before the supernatants were harvested and IFN-γ secretion was determined using ELISA and IFN-γ secretion was adjusted based on 1×10⁶ NK cells in the culture (FIG. 26A). One of three representative experiments is shown. OCs were generated from hu-BLT monocytes, as described in Material and Methods section. Allogeneic NK cells purified from healthy human-donors were pre-treated with IL-2 (1000 U/mL) and anti-CD16mAb (3 μg/mL) for 18 hours and then either cultured alone or with hu-BLT-OCs in the presence of sAJ2 (NK:OCs:sAJ2; 2:1:4) and the numbers of expanding NK cells were counted on days 6, 9, 12 and 15. At each day of culture, equal numbers of NK cells from each group were cultured and their cell growth was determined (FIG. 26B). On day 15 of the culture, cells were counted, and equal numbers of NK cells were used for cytotoxicity against OSCSCs using 4-hour ⁵¹Cr release assay. LUs 30/10⁶ cells were determined, as described in FIG. 19I (FIG. 26C). The supernatants from the NK and OC cultures in the presence of sAJ2, as described in FIG. 26C were harvested on days 6, 9, 12 and 15, the levels of IFN-γ were determined using ELISA (FIG. 26D). One of two representative experiments is shown.

FIG. 27A-FIG. 27E show that Hu-BLT NK cells expanded by hu-BLT and human derived osteoclasts secreted comparable levels of IFN-γ, and the levels were similar to those secreted by the NK cells from human donors expanded by the autologous osteoclasts on per cell basis.

Hu-BLT mice were implanted with tumors and injected with NK cells, as described in Materials and Methods, before spleen and BM were harvested and single cell suspensions were prepared after mice were euthanized. CD56+NK cells were positively selected from splenocytes, and monocytes were purified from BM cells, and co-cultured at (NK:Monocytes; 2:1 ratio) and treated with IL-2 (1000U/ml) alone or in combination with LPS (100 ng/mL) for 7 days before the supernatants were harvested and IFN-γ secretion was determined using ELISA (FIG. 27A). OCs were generated from the peripheral blood-derived monocytes of healthy human-donors and pancreatic cancer patients, as described in the Material and Methods section and were cultured with healthy human NK cells in the presence of sAJ2 and the numbers of NK cells were counted on days 6, 9, 12, 15, 18 and 22. On each day of culture, equal numbers of NK cells from each group were cultured and cell growth was determined (FIG. 27B). OCs were generated from the peripheral blood-derived monocytes of healthy human-donors and pancreatic cancer patients, as described in the Material and Methods section and were cultured with healthy human NK cells in the presence of sAJ2, and the numbers of NK cells were counted on days 6, 9, 12, 15, 18 and 22. On each day of culture, equal numbers of NK cells from each group were cultured. On day 15 of culture, NK cells were counted, and equal numbers of NK cells were used for cytotoxicity against OSCSCs using 4-hour ⁵¹Cr release assay. LUs 30/10⁶ cells were determined (FIG. 27C). Supernatants from the cultures were harvested on days 6, 9, 12, 15, 18 and 22 and the levels of IFN-γ were determined using single ELISA (FIG. 27D). One of three representative experiments is shown (FIGS. 27C and 27D). Surface expression of MHC-class I, CD54, KIR2, KIR3, KLRG1 and MICAS on the OCs of healthy donors and pancreatic cancer-patients were determined using antibody staining. One of three representative experiments is shown (FIG. 27E).

FIG. 28A-FIG. 28C show that identical amounts of IFN-γ secreted by cancer patient NK and T cells in comparison to healthy donor T cells induce lower levels of differentiation in oral cancer stem-like tumors. Supernatants containing equal amounts of IFN-γ from healthy donor and pancreatic patients' NK cells treated with IL-2 (1000 u/ml) and anti-CD16mAb (3 μg/ml) for 18 hours were added to OSCSCs for 4 days, to induce differentiation. Thereafter, expression of MHC-class I, B7H1 and CD54 were determined on the surface of OSCSCs (FIG. 28A). Allogeneic NK cells from healthy human donors were treated with IL-2 (1000 U/mL) for 18-24 hours before they were used in cytotoxicity against untreated and healthy and patient NK-supernatant differentiated OSCSCs, generated. Tumors were ⁵¹Cr labeled and used in the cytotoxicity assay, and LU 30/10⁶ cells were determined using inverse number of cells required to lyse 30% of OSCSCs×100 (FIG. 28B). One of three representative experiments is shown. Highly purified T cells from healthy donor and cancer patient were treated with IL-2 (100 U/mL) and anti-CD3/CD28 (1 μg/mL) for 18 hours before the supernatants were harvested and added to OSCSCs cultures for 4 days. Afterwards untreated OSCSCs and those treated with different T cell supernatants indicated in the figure, were detached from the tissue culture plates, extensively washed with 1×PBS, and labeled with ⁵¹Cr. Freshly isolated allogeneic NK from healthy human donor cells were treated with IL-2 (1000 U/mL) for 24 hours before the cells were used as effector cells in ⁵¹Cr release assay against OSCSCs treated with T cells supernatants. The lytic units 30/10⁶ cells were determined using inverse number of NK cells required to lyse 30% of the target cells×100 (FIG. 28C). One of the two representative experiment is shown in the figure.

FIG. 29A-FIG. 29E show that osteoclasts generated from Hu-BLT monocytes induced expansion and increased the cytokines secretion in the NK cells isolated from Hu-BLT splenocytes. Osteoclasts (OCs) were generated from human peripheral blood isolated monocytes as described in Material and Methods section. Allogeneic NK cells purified from healthy human-donors were pre-treated with IL-2 (1000 U/mL) and anti-CD16mAb (3 μg/mL) for 18 hours and then either cultured alone or with human OCs in the presence of sAJ2 (NK:OCs:sAJ2; 2:1:4) and the numbers of expanding NK cells were counted on days 6, 10, 14, 18 and 22. At each day of culture equal numbers of NK cells from each group were cultured and cell growth determined (FIG. 29A). One of the three representative experiment is shown in the figure. OCs were generated from hu-BLT bone marrow monocytes and human peripheral blood monocytes as described in Material and Methods section. NK cells purified from hu-BLT splenocytes were pre-treated with IL-2 (1000 U/mL) and anti-CD16mAb (3 μg/mL) for 18 hours and then either cultured alone or with hu-BLT-OCs or human OCs in the presence of sAJ2 (NK:OCs:sAJ2; 2:1:4) and the numbers of expanding NK cells were counted on days 6, 10, 14, 18 and 22. At each day of culture equal numbers of NK cells from each group were cultured and cell growth determined (FIG. 29B). One of the three representative experiment is shown in the figure. The supernatants from the NK cells and OC cultures in the presence of sAJ2, as described in FIG. 29A were harvested on days 6, 10, 14, 18 and 22, the levels of IFN-γ were determined using single ELISA (FIG. 29C). The supernatants from the NK cells and OCs cultures in the presence of sAJ2 were harvested on days 6, 10, 14, 18 and 22, the levels of IFN-γ were determined using single ELISA (FIG. 29D). IFN-γ secretion in FIGS. 29C and 29D was adjusted based on 1×10⁶NK cells (NK cells counts in FIGS. 29A and 29B in the culture) (FIG. 29E). One of the three representative experiment is shown in FIGS. 29D and 29E.

FIG. 30A-FIG. 30E show decreased numbers of PBMCs and functional loss of NK cells obtained from peripheral blood of cancer patients. Human PBMCs were isolated from the peripheral blood (30 ml) of the healthy individuals (n=14) and cancer patients (n=14), and the numbers of cells were determined using microscopy (FIG. 30A). Human PBMCs were isolated from the peripheral blood of the healthy individuals (n=9-12) and cancer patients (n=9-12), and equal numbers (2×10⁵ cells) of PBMCs were used to determine the percentages of CD16 (n=12), CD56 (n=12), CD3 (n=12), CD19 (n=9), CD14 (n=10) and CD11b (n=9) subsets within CD45+ immune cells using flow cytometric analysis (FIG. 30B). Human NK cells were isolated from healthy individuals (n=9) and cancer patient's (n=9) PBMCs as described in Materials and Methods. Purified NK cells (1×10⁶ cells/ml) were left untreated and treated with IL-2 (1000 U/ml) for 18 hours before the supernatants were harvested and IFN-γ secretion was determined using single ELISA (FIG. 30C). NK cells isolated and treated as described in FIG. 30C, and were added to ⁵¹Cr labeled oral squamous cell carcinoma stem cells (OSCSCs) at various effectors to target ratios. NK cell-mediated cytotoxicity using a standard 4-hour ⁵¹Cr release assay against the oral squamous cell carcinoma stem cell line (OSCSCs). The lytic units 30/10⁶ cells were determined using the inverse number of lymphocytes required to lyse 30% of OSCSCs×100 (n=9 for each experiment condition) (FIG. 30D). Highly purified NK cells isolated from individuals (n=4) and cancer patients' (n=4) PBMCs were treated (1×10⁶ cells/ml) with IL-2 (1000 U/ml) for 18 hours before the supernatants were harvested and ran with multiplex cytokine array kit to determine IFN-γ, IL-12p70, IL-6, TNF-α, IL-5 and IL-4 secretion (FIG. 30E).

FIG. 31 shows decreased cytokines, chemokine and growth factors secretions in the serum of cancer patients. Sera were obtained from peripheral blood of healthy donors (n=5) or cancer patients (n=8) and analyzed for the levels of cytokines, chemokine and growth factors using multiplex array kit.

FIG. 32A-FIG. 32I show that osteoclast-modulated NK cells from cancer patients have much lower capacity to expand, or mediate cytotoxicity and secrete IFN-γ compared to healthy individuals. Monocytes were purified from healthy individual PBMCs and cultured in alpha-MEM media containing M-CSF (25 ng/ml) and RANKL (25 ng/ml) for 21 days to generate osteoclasts (OCs). Purified NK cells (1×106 cells/ml) from healthy individuals (n=70) and cancer patients (n=70) were treated with the combination of IL-2 (1000 U/ml) and anti-CD16mAb (3 μg/ml) for 18 hours before they were cultured with OCs generated from healthy individual monocytes in the presence of sAJ2 at 1:2:4 ratios (OCs:NK:sAJ2). After 6, 9, 12 and 15 days of co-culture, the numbers of expanded lymphocytes were determined using microscopy (FIG. 32A). Freshly purified NK cells from the healthy individuals (n=16) and cancer patients (n=16) were treated and co-cultured with OCs as described in FIG. 32A. Cytotoxicity of day 15 cultured NK cells was determined using standard 4-hour ⁵¹Cr release assay against OSCSCs. The lytic units 30/10⁶ cells were determined using the method described in FIG. 30D (FIG. 32B). Freshly purified NK cells from the healthy individuals (n=63) and cancer patients (n=63) were treated and co-cultured with OCs as described in FIG. 32A, and the supernatants were harvested from the days 6, 9, 12 and 15 co-cultures and IFN-γ secretion was determined using single ELISAs (FIG. 32C). Freshly purified NK cells from the healthy individuals (n=21) and cancer patients (n=21) were treated and co-cultured with OCs as described in FIG. 32A. After 6, 9, 12 and 15 days of co-cultures, the numbers of expanded NK cells were counted using microscopy and the supernatants were harvested to determine the IFN-γ secretion using single ELISA. IFN-γ secretion was assessed based on per 1 million cell counts (FIG. 32D). Purified T cells (1×10⁶ cells/ml) from healthy individuals (n=70 and cancer patients (n=7) were treated with the combination of IL-2 (100 U/ml) and anti-CD3 (1 μg/ml)/CD28mAb (3 μg/ml) for 18 hours before they were co-cultured with OCs in the presence of sAJ2 at 1:2:4 ratios (OCs:T:sAJ2). After 6, 9, 12 and 15 days of co-culture, lymphocytes were manually counted using microscopy, the cumulative cell counts of lymphocytes from day 0-day 15 is shown in FIG. 32E. Purified T cells healthy individuals (n=42) and cancer patients (n=42) were treated and co-cultured with OCs as described in FIG. 32E. The supernatants were harvested from the day 6, 9, 12 and 15 co-cultures and IFN-γ secretion was determined using single ELISAs (FIG. 32F). Purified T cells from the healthy individuals (n=28) and cancer patients (n=28) were treated and cultured with OCs as described in FIG. 32E. After 6, 9, 12 and 15 days of co-culture, lymphocytes were manually counted using microscopy and the supernatants were harvested from the co-cultures to determine IFN-γ secretion using single ELISAs and normalized per million lymphocytes counts (FIG. 32G). Freshly purified NK cells from the healthy individuals (n=10) were treated and co-cultured with OCs as described in FIG. 32A. Purified T cells from the healthy individuals (n=10) were treated and cultured with OCs as described in FIG. 32E. After 6, 9, 12 and 15 days of co-culture, lymphocytes were manually counted using microscopy, the cumulative cell counts of lymphocytes from day 0-day 15 is shown in the figure (FIG. 32H). Freshly purified NK cells from the healthy individuals (n=10) were treated and co-cultured with OCs as described in FIG. 32A. Purified T cells from the healthy individuals (n=10) were treated and cultured with OCs as described in FIG. 32E. The supernatants were harvested on day 6, 9, 12 and 15 of the co-cultures, IFN-γ secretion was determined using single ELISAs, the cumulative IFN-γ secretion by the lymphocytes from day 0-day 15 is shown in figure (FIG. 32I).

FIG. 33A-FIG. 33C show significantly continuous decreases of IFN-γ secretion by osteoclast-expanded patient's NK cells when compared to those obtained from healthy individuals' NK cells in different days of expansion. Purified NK cells (1×10⁶ cells/ml) from healthy individuals (n=8) and cancer patients (n=8) were treated with the combination of IL-2 (1000 U/ml) and anti-CD16mAb (3 μg/ml) for 18 hours before they were treated with sAJ2 at 1:2 ratios (NK:sAJ2). The supernatants were harvested from the day 6, 9, 12 and 15 co-cultures and IFN-γ secretion was determined using single ELISA (FIG. 33A). Monocytes were purified from healthy individuals PBMCs and, were cultured in alpha-MEM media containing M-CSF (25 ng/ml) and RANKL (25 ng/ml) for 21 days to generate osteoclasts. Purified NK cells (1×10⁶ cells/ml) from the healthy individuals (n=8) and cancer patients (n=8) were treated with the combination of IL-2 (1000 U/ml) and anti-CD16mAb (3 μg/ml) for 18 hours before they were co-cultured with sAJ2 and OCs at 1:2:4 ratios (OCs:NK:sAJ2). The supernatants were harvested from the day 6, 9, 12 and 15 co-cultures and IFN-γ secretion was determined using single ELISA (FIG. 33B). Purified NK cells from the healthy individuals and cancer patients were treated and cultured with OCs as described in FIG. 33B. IFN-γ secretion was determined using ELISPOT assay on day 21 of co-culture. One of the 3 representative experiments is shown in the figure (FIG. 33C).

FIG. 34A-FIG. 34G show significantly decreased IFN-γ secretion by primary, non-osteoclast expanded and osteoclast-expanded patient's T cells when compared to T cells from the healthy individuals' T cells in different days of expansion. Monocytes were purified from human PBMCs and cultured in alpha-MEM media containing M-CSF (25 ng/ml) and RANKL (25 ng/ml) for 21 days to generate osteoclasts. Positively selected T cells (1×10⁶ cells/ml) from healthy individuals (n=4) and cancer patients (n=4) were treated with the combination of IL-2 (100 U/ml) and anti-CD3 (1 μg/ml) for 18 hours before they were treated with sAJ2 in the absence or presence of OCs at 1:2:4 ratios (OCs:T:sAJ2). After 6, 9, 12 and 15 days of co-cultures, the numbers of expanded T cells were counted using microscopy, the cumulative cell counts of lymphocytes from day 0-day 15 is shown in the figure (FIG. 34A). Purified T cells from the healthy individuals (n=4) and cancer patients (n=4) were treated and cultured with OCs as described in FIG. 34A. The supernatants were harvested on day 6, 9, 12 and 15 of the co-cultures, IFN-γ secretion was determined using single ELISAs, the cumulative IFN-γ secretion by the lymphocytes from day 0-day 15 is shown in figure (FIG. 34B). Purified T cells from the healthy individuals (n=15) and cancer patients (n=15) were treated and cultured with OCs as described in FIG. 34A. The supernatants were harvested on day 6, 9, 12 and 15 of the co-cultures, IFN-γ secretion was determined using single ELISAs, the cumulative IFN-γ secretion by the lymphocytes from day 0-day 15 is IFN-γ was assessed based on 1 million cell counts of cumulative cell counts of lymphocytes from day 0-day 15 (FIG. 34C). Purified T cells from healthy individuals (n=4) and cancer patients (n=4) were treated with the combination of IL-2 (100 U/ml) and anti-CD3 (1 μg/ml) for 18 hours before they were treated with sAJ2 at 1:2 ratios (T:sAJ2). The supernatants were harvested from the day 6, 9, 12 and 15 co-cultures and IFN-γ secretion was determined using single ELISA (FIG. 34D). Purified T cells from the healthy individuals (n=4) and cancer patients (n=4) were treated and cultured with OCs as described in FIG. 34A. The supernatants were harvested from the day 6, 9, 12 and 15 co-cultures and IFN-γ secretion was determined using single ELISA (FIG. 34E). Purified T cells from the healthy individuals (n=3) and cancer patients (n=3) were treated and cultured with OCs as described in FIG. 34A. IFN-γ secretion was determined using ELISPOT assay on day 21 of co-culture (FIG. 34F). Freshly purified NK cells from the healthy individuals (n=4) were treated and co-cultured with and without OCs as described in FIGS. 33A and 33B. Positively selected T cells from the healthy individuals (n=4) were treated and cultured with and without OCs as described in FIGS. 34A and 34D. After 6, 9, 12 and 15 days of co-culture, lymphocytes were manually counted using microscopy, the cumulative cell counts of lymphocytes from day 0-day 15 is shown in the figure (FIG. 34G).

FIG. 35A-FIG. 35E shows decreased percentages of CD4+ T cells and an increase in percentages of CD8+ T cells when determined within cancer patients' PBMCs as compared to healthy individuals' PBMCs. Freshly isolated T cells from the healthy individuals (n=12) and cancer patients (n=12) PBMCs were analyzed for the surface expressions of CD45RO, CD45RA. CD62L, CD28, CCR7, and CD127 using flow cytometry. IgG2 isotype was used as control (FIG. 35A). Human PBMCs were isolated from peripheral blood of the healthy individuals (n=12) and cancer patients (n=12), surface expression of CD4 and CD8 were analyzed within CD3+ immune cells using flow cytometry (FIG. 35B). Human PBMCs were isolated from peripheral blood of the healthy individuals (n=12) and cancer patients (n=12), surface expression of CD4 and CD8 were analyzed within CD3+ immune cells using flow cytometry. The proportion of CD4+ T cells were compared with the proportion of CD8+ T cells and ratio is shown in figure (FIG. 35C). Freshly purified NK cells from the healthy individuals (n=28) and cancer patients (n=28) were treated and co-cultured with OCs as described in FIG. 32A. Purified T cells from the healthy individuals (n=28) and cancer patients (n=28) were treated and cultured with OCs as described in FIG. 32E. After 6, 9, 12 and 15 days of co-culture, surface expression of CD4 and CD8 were analyzed within CD3+ immune cells using flow cytometry (FIG. 35D), and the proportion of CD4+ T cells were compared with the proportion of CD8+ T cells and ratio is shown in figure (FIG. 35E).

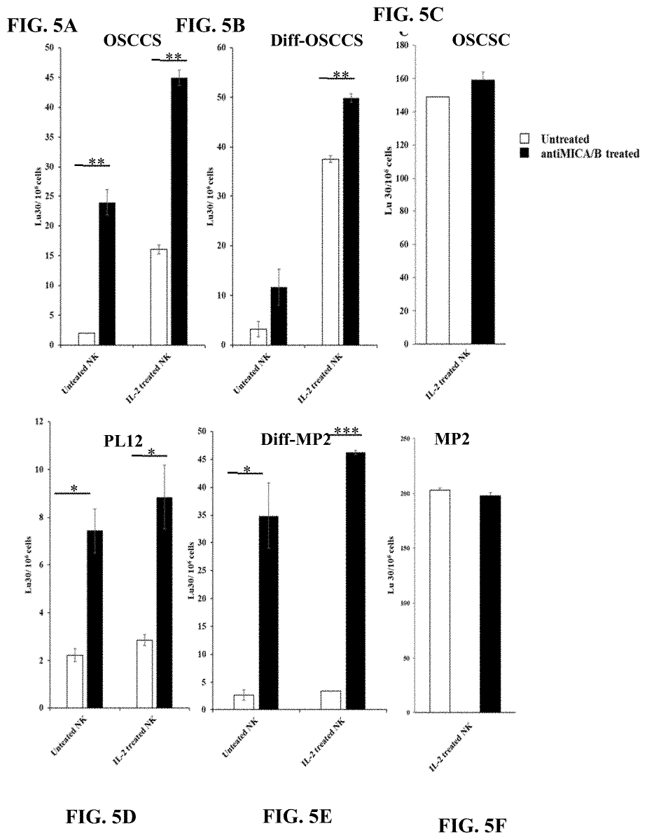

FIG. 36A-FIG. 36M shows that osteoclast-modulated NK cells promote NK cell expansion, whereas dendritic cells-modulated NK cells promote T cells expansion; OC-expanded NK cells exhibit higher cytotoxic function compared to DC-expanded NK cells. Monocytes were purified from healthy individual PBMCs and were cultured with GM-CSF (150 ng/ml) and IL-4 (50 ng/ml) for 8 days to generate DCs. To generate osteoclasts, monocytes were cultured as described in FIG. 32A. For expansion, purified NK cells from healthy individuals (1×10⁶ cells/ml) were treated with the combination of IL-2 (1000 U/ml) and anti-CD16mAb (3 μg/ml) for 18 hours before they were co-cultured with autologous DCs or OCs in the presence of sAJ2 at 1:2:4 ratios (DCs or OCs:NK:sAJ2). The numbers of expanded lymphocytes were assessed at days 8, 11, 15 and 18 using microscopic determination (n=30 for each experiment condition) (FIG. 36A). NK cells were co-cultured with OCs or DCs as described in FIG. 38A, surface expression of CD3, CD16, and CD56 was analyzed at days 8, 11, 15 and 18. The numbers of NK cells (FIG. 36B) and T cells (FIG. 36C) cells were determined using the percentages of CD16+ and CD3+ surface expression respectively within the total expanding cells in FIG. 36A (n=30 for each experiment condition) (FIGS. 36B and 36C). NK cells were co-cultured with OCs or DCs as described in FIG. 38A and cytotoxicity were determined on the day 15 using a standard 4-hour 51Cr release assay against OSCSCs. The lytic units 30/10⁶ cells were determined using the method described in FIG. 30D (n=12 for each experiment condition) (FIG. 36D). NK cells were co-cultured with OCs or DCs as described in FIG. 38A, surface expression of CD16 on day 15. The LUs from FIG. 36D was used to assess the LUs based on 1% of NK cells (n=12 for each experiment condition) (FIG. 36E). NK cells were co-cultured with OCs or DCs as described in FIG. 38A, the supernatants were harvested on day 8, 11, 15 and 18 of the co-cultures, IFN-γ secretion was determined using single ELISA (n=12 for each experiment condition) (FIG. 36F). NK cells were co-cultured with OCs or DCs as described in FIG. 5A, the numbers of CD3+CD4+ cells (FIG. 36G) and CD3+CD8+ cells (FIG. 36H) cells were determined using the surface expression percentages of CD4+ and CD8+ in CD3+ cells respectively within the total expanding cells on days as shown in the figures (n=12 for each experiment condition) (FIGS. 36G and 36H). NK cells were co-cultured with OCs or DCs as described in FIG. 38A, the surface expression of CD3, CD4, and CD8 was analyzed at days 8, 11, 15 and 18, CD4 and CD8 percentages are shown in figure (n=12 for each experiment condition) (FIG. 36I). NK cells were co-cultured with OCs or DCs as described in FIG. 38A. Surface expressions of CD4, CD8, KLRG1, TIM3, and PD-1 were analyzed within CD3+ cells on day 27 of the co-culture (n=8 for each experiment condition) (FIG. 36J). DCs and OCs were generated as described in FIG. 38A. Purified T cells (1×10⁶ cells/ml) from the healthy individuals were treated with the combination of IL-2 (100 U/ml) and anti-CD3 (1 μg/ml)/CD28mAb (3 μg/ml) for 18 hours before they were co-cultured with autologous DCs or OCs in the presence of sAJ2 at 1:2:4 ratios (DCs or OCs:T:sAJ2). Surface expressions of CD4, CD8, KLRG1, TIM3 and PD-1 were analyzed within CD3+ cells on day 27 of the co-culture (n=8 for each experiment condition) (FIG. 36K). DCs and OCs were generated, NK Cells were co-cultured with OCs or DCs as described in FIG. 38A, and T cells were treated and co-cultured with OCs or DCs. Surface expressions of CD45RO, CD62L, CD28, CD44, CCR7 and CD127 were analyzed within CD3+ cells on day 12 of the co-culture (n=8 for each experiment condition) (FIG. 36L) and surface expressions of CD3, CD16. CD56, CD4, CD8, CD28 and CD62L were analyzed day 12 of the co-culture (n=8 for each experiment condition) (FIG. 36M).

FIG. 37A-FIG. 37G show that osteoclast expanded NK cells secrete more cytokines and chemokines when compared to OC-expanded T cells either modulated by NK cells or expanded alone with OCs. Freshly purified NK cells from the healthy individuals were treated and co-cultured with OCs as described in Fig. S2B. On day 12 of the culture, NK cells, CD3+ T cells, CD4+ T cells and CD8+ T cells were isolated using specific isolation kits for each cell types. Isolated NK cells treated with the combination of IL-2 (1000 U/ml) and anti-CD16mAb (3 μg/ml), and each T cells subtype were treated with IL-2 (100 U/ml) and anti-CD3 (1 μg/ml)/CD28mAb (3 μg/ml) for 18 hours before the supernatants were harvested and analyzed for the levels of cytokines, chemokines and growth factors using multiplex array kit. The levels of secretions from each cell types were adjusted based on 1 million cell counts, fold increase of secretion levels of CD3+, CD4+, and CD8+ T cells from the secretion levels of NK cells is shown in the figures (FIGS. 37A and 37B). Freshly purified NK cells from the healthy individuals were treated and co-cultured with OCs as described in FIG. 32B, and freshly purified T cells from the healthy donors were treated and co-cultured with OCs as described in FIG. 34A. Day 6 after the culture, the supernatants were harvested and analyzed for the levels of cytokines, chemokines and growth factors using multiplex array kit, fold increase of secretion levels of T cells from the secretion levels of NK cells is shown in the figures (FIGS. 37C and 37E). The levels of secretions in FIGS. 37C and 37E were adjusted based on 1 million cell counts, fold increase of secretion levels of T cells from the secretion levels of NK cells is shown in the figures (FIGS. 37D and 37F). Freshly purified NK cells from the healthy individuals were treated and co-cultured with OCs as described in FIG. 33B, on day 12 of the culture, CD8+ T cells were isolated using isolation kits, and were treated with IL-2 (100 U/ml) and anti-CD3 (1 μg/ml)/CD28mAb (3 μg/ml) for 18 hours (culture A). In separate culture freshly isolated CD8+ T cells purified from healthy individuals PBMCs were treated with IL-2 (100 U/ml) and anti-CD3 (1 μg/ml)/CD28mAb (3 μg/ml) for 18 hours before they were cultured with OCs (1:2:4; OCs:T:sAJ2). On day 12 of culture, CD8+ T cells were isolated from the culture using isolation kits, and were treated with IL-2 (100 U/ml) and anti-CD3 (1 μg/ml)/CD28mAb (3 μg/ml) for 18 hours (culture B). The supernatants were harvested from both culture A and culture B, CD8+ T cell cultures at same time and analyzed for the levels of cytokines, chemokines and growth factors using multiplex array kit, and the fold increase of secretion levels of CD8+ T cells sorted from OCs expanded NK cells from the secretion levels of CD8+ T cells cultured with OCs is shown in the figures (FIG. 37G). One of the 3 representative experiments is shown in FIG. 37.

FIG. 38A-FIG. 38J show that immunotherapy with OC-expanded NK cells in tumor-bearing hu-BLT mice increased CD8+ T cells in BM, spleen, and peripheral blood and, resulted in increased IFN-γ secretion and elevated NK cell-mediated cytotoxicity in those tissue compartments. Reconstituted BLT (levels and lineages of T cells comparable to the healthy donors) were orthotopically injected with 1×10⁶ of human OSCSCs into the floor of the mouth. 1-2 weeks after the tumor implantation, mice were i.v injected with day 12 OC-expanded human NK cells, NK cells were treated and co-cultured with OCs as described in FIG. 32A. Disease progression and mice weight loss was monitored for another 3-4 weeks (n=3) (FIG. 38A). At the end of experiment hu-BLT mice were sacrificed, the spleens, BM and peripheral blood were harvested, and single cell suspensions were obtained as described in Materials and Methods, and cells obtained from each tissue were treated with IL-2 (1000 U/ml) and cultured for 7 days. Surface expression of CD3 and CD8 was analyzed at days 7 of BM culture (n=3 for each experimental condition) (FIG. 38B), spleen culture (n=3 for each experimental condition) (FIG. 38E), PBMC culture (n=2 for each experimental condition) (FIG. 38H). The supernatants were harvested from the cultures on day 7 of BM culture (n=3 for each experimental condition) (FIG. 38C), spleen culture (n=3 for each experimental condition) (FIG. 38F), PBMC culture (n=2 for each experimental condition) (FIG. 38I), and IFN-γ secretion was determined using single ELISA (FIGS. 38C, 38F, and 38I). NK-cells mediated cytotoxicity against OSCSCs of 7 days BM culture (n=3 for each experimental condition) (FIG. 38D), spleen culture (n=3 for each experimental condition) (FIG. 38G), PBMC culture (n=2 for each experimental condition) (FIG. 38J) was determined using standard 4-hour ⁵¹Cr release assay against OSCSCs. The lytic units 30/10⁶ cells were determined using the method described in FIG. 30D (FIGS. 38D, 38G, and 38J).

FIG. 39A-FIG. 39E show that immunotherapy with OC-expanded NK cells in tumor-bearing hu-BLT mice increased cytokine secretion in the serum. Reconstituted hu-BLT (levels and lineages of T cells comparable to the healthy donors) were orthotopically injected with 1×10⁶ of human OSCSCs into the floor of the mouth. 1-2 weeks later hu-BLT mice were injected i.v with 1.5×10⁶ of OC-expanded human super-charged NK cells, NK cells were treated and co-cultured with OCs as described in FIG. 33B, day 12 expanded NK cells were used for mice injections. Disease progression and weight loss was monitored for another 3-4 weeks, after which mice were sacrificed, peripheral blood was collected in heparin-free vials post-mortem by cardiac puncture and serum samples were harvested and analyzed for IFN-γ (FIG. 39A), IL-6 (FIG. 39B), ITAC (FIG. 39C), IL-8 (FIG. 39D) and GM-CSF (FIG. 39E) secretion using multiplex arrays (FIGS. 39A-39E). One of the 3 representative experiments is shown in FIG. 39.

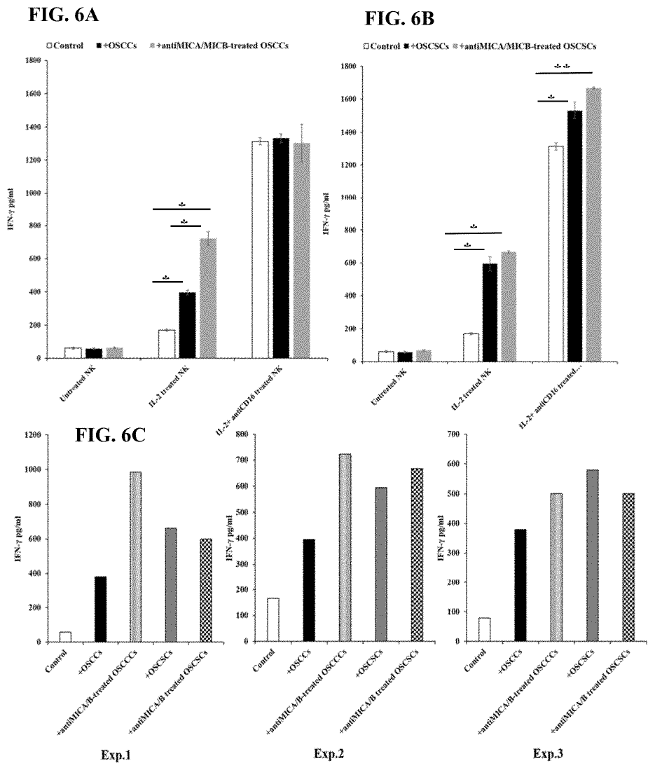

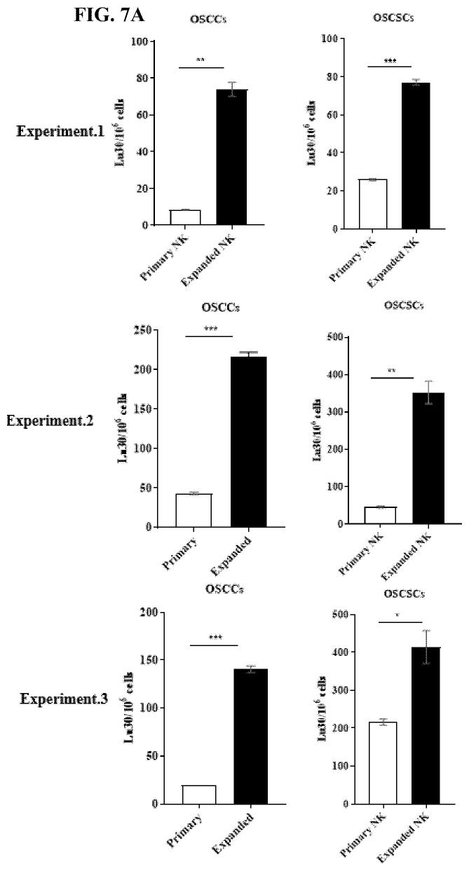

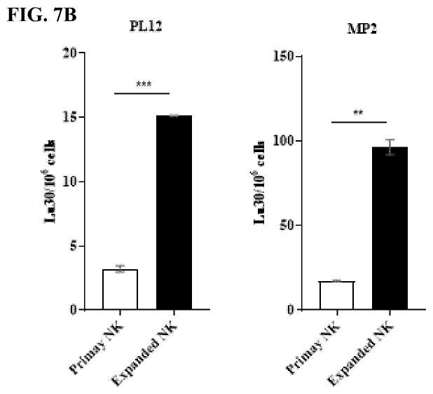

FIG. 40A-FIG. 40F show that the gradual increase in the numbers of CD8+ T cells when they were cultured with OCs whereas the number of CD4+ T cells decreases substantially indicating that OCs preferentially supports the expansion of CD8+ T cells. Freshly purified CD8+ T cells and CD4+ T cells (1×10⁶ cells/ml) from the healthy individuals were treated with the combination of IL-2 (100 U/ml) and anti-CD3 (1 μg/ml)/CD28mAb (3 μg/ml) for 18 hours before they were co-cultured with sAJ2 (T:sAJ2; 1:2). After 6, 12, 15 and 19 days of co-culture, lymphocytes were manually counted using microscopy, fold expansion for each time points are shown as indicated in figure (n=6 for each experiment condition) (FIG. 40A). Purified CD8+ T cells and CD4+ T cells from the healthy individuals were treated as described in FIG. 6A for 18 hours before they were co-cultured with OCs in the presence of sAJ2 at 1:2:4 ratios (OCs:CD4TorCD8T:sAJ2). After 6, 12, 15 and 19 days of co-culture, lymphocytes were manually counted using microscopy, fold expansion for each time points are shown as indicated in figure (n=6 for each experiment condition) (FIG. 40B). Purified NK cells from the healthy individuals were treated and co-cultured with OCs as described is FIG. 32A. Purified CD8+ T cells and CD4+ T cells from the healthy individuals were treated as described in FIG. 40A. After 6, 9, 12 and 15 days of co-culture, lymphocytes were manually counted using microscopy, the numbers of OC expanded NK, CD4+ T and CD8+ T cells were subtracted from the number of non-OC expanded control cells, and fold expansion of the cells were determined by dividing it to the initial input cells (n=6 for each experiment condition) (FIG. 40C). Purified NK cells from the healthy individuals were treated and co-cultured with OCs as described is FIG. 32A. Purified CD8+ T cells and CD4+ T cells from the healthy individuals were treated as described in FIG. 40A. The supernatants were harvested from the day 6, 9, 12 and 15 co-cultures, IFN-γ secretion was determined using single ELISA and IFN-γ secreted was adjusted per 1 million of lymphocytes (n=3 for each experiment condition) (FIG. 40D). Freshly purified CD4+ T and CD8+ T cells from healthy individuals were treated with anti-CD3 (1 μg/ml) and IL-2 (100 U/ml) for 18 hours, and freshly purified NK cells from healthy individuals were treated with the combination of IL-2 (1000 U/ml) and anti-CD16mAb (3 μg/ml) for 18 hours before NK cells were used to determine NK-cells mediated cytotoxicity against CD4+ T cells and CD8+ T cells using TVA assay and, the lytic units 30/107 cells were determined using inverse number of NK cells required to lyse 30% of target cells×100 (FIG. 40E). Freshly purified CD4+ T and CD8+ T cells from healthy individuals were treated with IL-2 (100 U/ml) for 18 hours, and freshly purified NK cells from healthy individuals were treated as described in FIG. 40E before NK cells were used to determine NK-cells mediated cytotoxicity against CD4+ T cells and CD8+ T cells using TVA assay and, the lytic units 30/107 cells were determined using inverse number of NK cells required to lyse 30% of target cells×100 (FIG. 40F).

FIG. 41 shows that NK cells preferentially lyse CD4+ T cells and not CD8+ T cells. Freshly purified CD4+ T and CD8+ T cells from healthy individuals were treated with anti-CD3 (1 μg/ml) and IL-2 (100 U/ml) for 18 hours, and freshly purified NK cells from healthy individuals were treated with the combination of IL-2 (1000 U/ml) for 18 hours before NK cells were used to determine NK-cells mediated cytotoxicity against CD4+ T cells, CD8+ T cells AND OSCSCs using TVA assay and, the lytic units 30/10⁷ cells were determined using inverse number of NK cells required to lyse 30% of target cells×100.

### DETAILED DESCRIPTION OF THE INVENTION

The present invention relates, in part, to immunological compositions and methods of using them to treat a subject afflicted with a cancer, and/or to kill or inhibit proliferation of cancer cells. The compositions comprises multiple immune cell types that can target different populations of cancer cells. Such compositions can also benefit diseases other than cancer, as they can generally strengthen the immune system of a subject.

### I. Definitions

The articles “a” and “an” are used herein to refer to one or to more than one (i.e. to at least one) of the grammatical object of the article. By way of example, “an” element means one element or more than one element.

The term “administering” is intended to include routes of administration which allow an agent to perform its intended function. Examples of routes of administration for treatment of a body which can be used include injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier.

The term “activating” or “activation” refers to an enhancement of the function of a target. For example, the instant disclosure provides a method of activating a NK cell in vitro, ex vivo, and/or in vivo. In the instant disclosure, the activation of a cell refers to an enhancement of the function of such cell, including at least an enhancement of activity and/or at least one cellular function (e.g., cytotoxicity, cell division and/or growth rate, etc.). In some embodiments, the agent used herein activates at least one cell, such as NK cell(s).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. Unless otherwise stated, the terms include metaplasias. In some embodiments, such cells exhibit such characteristics in part or in full due to at least one genetic mutations. Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is oral cancer, oral squamous carcinoma, breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The term “control” refers to any suitable reference standard, such as a normal patient, cultured primary cells/tissues isolated from a subject such as a normal subject, adjacent normal cells/tissues obtained from the same organ or body location of the patient, a tissue or cell sample isolated from a normal subject, or a primary cells/tissues obtained from a depository. In other preferred embodiments, the control may comprise an expression level, numbers of a certain cell type (e.g., NK cells or monocytes), and/or a cellular function of a certain cell type for a set of subject, such as a normal or healthy subject.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

The term “cytokine” refers to a broad and loose category of small proteins (˜5-20 kDa) that are important in cell signaling. Their release has an effect on the behaviour of cells around them. cytokines are involved in autocrine signaling, paracrine signaling and endocrine signaling as immunomodulating agents. Cytokines include chemokines, interferons, interleukins, lymphokines, and tumour necrosis factors, and may additionally include hormones or growth factors in the instant disclosure. Cytokines are produced by a broad range of cells, including immune cells like macrophages, B lymphocytes, T lymphocytes and mast cells, as well as endothelial cells, fibroblasts, and various stromal cells. Preferred cytokines are exemplified in the specification and the Figures of the instant disclosure.

The term “cytokine/chemokine activity,” includes the ability of a cytokine or a chemokine to modulate at least on of cellular functions. Generally, cytokines or chemokines modulate the balance between humoral and cell-based immune responses, and they regulate the maturation, growth, and responsiveness of particular cell populations. Thus, the term “cytokine/chemokine activity” includes the ability of a cytokine or chemokine to bind its natural cellular receptor(s), the ability to modulate cellular signals, and the ability to modulate the immune response.

The term “immune response” includes NK-mediated, T cell mediated, and/or B cell mediated immune responses. Exemplary immune responses include T cell responses, e.g., cytokine production and cellular cytotoxicity. In addition, the term immune response includes immune responses that are indirectly affected by NK cell or T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The instant disclosure provides methods to activate NK cells. The term “activate”, “activation,” or “activating” refers to activating NK cell functions. The term “NK cell function(s)” refers to any function of NK cells, such as cytotoxicity and/or cytokine/chemokine production/secretion activities, especially secretion of IFN-γ.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “inhibit” includes the reduce, decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

The term “subject” refers to any healthy animal, mammal or human, or any animal, mammal or human afflicted with a cancer, e.g., brain metastasis, oral cancer, lung, ovarian, pancreatic, liver, breast, prostate, colon carcinomas, melanoma, multiple myeloma, and the like. The term “subject” is interchangeable with “patient.”

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

Unless otherwise specified here within, the terms “antibody” and “antibodies” refers to antigen-binding portions adaptable to be expressed within cells as “intracellular antibodies.” (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

Antibodies may be polyclonal or monoclonal; xenogeneic, allogeneic, or syngeneic; or modified forms thereof (e.g., humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized”, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

Since it is well-known in the art that antibody heavy and light chain CDR3 domains play a particularly important role in the binding specificity/affinity of an antibody for an antigen, the recombinant monoclonal antibodies of the present invention prepared as set forth above preferably comprise the heavy and light chain CDR3s of variable regions of the antibodies described herein and well-known in the art. Similarly, the antibodies can further comprise the CDR2s of variable regions of said antibodies. The antibodies can further comprise the CDR1s of variable regions of said antibodies. In other embodiments, the antibodies can comprise any combinations of the CDRs.

The CDR1, 2, and/or 3 regions of the engineered antibodies described above can comprise the exact amino acid sequence(s) as those of variable regions of the present invention described herein. However, the ordinarily skilled artisan will appreciate that some deviation from the exact CDR sequences may be possible while still retaining the ability of the antibody, especially an introbody, to bind a desired target, either alone or in combination with an immunotherapy, such as the one or more biomarkers, the binding partners/substrates of such biomarkers, or an immunotherapy effectively (e.g., conservative sequence modifications). Accordingly, in other embodiments, the engineered antibody may be composed of one or more CDRs that are, for example, 50%, 60%, 70%, 80%, 85%, 90%, 91%, 92%, 93%, 94%, 95%, 96%, 97%, 98%, 99%, or 99.5% identical to one or more CDRs of the present invention described herein or otherwise publicly available.

For example, the structural features of non-human or human antibodies (e.g., a rat anti-mouse/anti-human antibody) can be used to create structurally related human antibodies, especially introbodies, that retain at least one functional property of the antibodies of the present invention, such as an immune checkpoint. Another functional property includes inhibiting binding of the original known, non-human or human antibodies in a competition ELISA assay.

Antibodies, immunoglobulins, and polypeptides of the invention can be used in an isolated (e.g., purified) form or contained in a vector, such as a membrane or lipid vesicle (e.g. a liposome). Moreover, amino acid sequence modification(s) of the antibodies described herein are contemplated. For example, it may be desirable to improve the binding affinity and/or other biological properties of the antibody. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce binding activity and can be corrected by replacing the amino acids with amino acid residues of the original antibody derived from a non-human animal.

Similarly, modifications and changes may be made in the structure of the antibodies described herein, and in the DNA sequences encoding them, and still obtain a functional molecule that encodes an antibody and polypeptide with desirable characteristics. For example, antibody glycosylation patterns can be modulated to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagines-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr et al. (1987) and by Edge et al. (1981). Enzymatic cleavage of carbohydrate moieties on antibodies can be achieved by the use of a variety of endo- and exo-glycosidases as described by Thotakura et al. (1987).

The term “immunotherapy” or “immunotherapies” refer to any treatment that uses certain parts of a subject's immune system to fight diseases such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

The term “immunogenic chemotherapy” refers to any chemotherapy that has been demonstrated to induce immunogenic cell death, a state that is detectable by the release of one or more damage-associated molecular pattern (DAMP) molecules, including, but not limited to, calreticulin, ATP and HMGB1 (Kroemer et al. (2013), Annu. Rev. Immunol., 31:51-72). In addition, the term “immunogenic chemotherapy” further refers to any chemotherapy that results in priming the immune system such that it leads to enhanced immune activity towards cancer. Specific representative examples of consensus immunogenic chemotherapies include 5′-fluorouracil, anthracyclines, such as doxorubicin, and the platinum drug, oxaliplatin, among others.

In some embodiments, immunotherapy comprises inhibitors of one or more immune checkpoints. The term “immune checkpoint” refers to a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICO S, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR (see, for example, WO 2012/177624). The term further encompasses biologically active protein fragments, as well as nucleic acids encoding full-length immune checkpoint proteins and biologically active protein fragments thereof. In some embodiments, the term further encompasses any fragment according to homology descriptions provided herein. In certain embodiments, the immune checkpoint is PD-1.

Immune checkpoints and their sequences are well-known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

The nucleic acid and amino acid sequences of a representative human PD-1 biomarker is available to the public at the GenBank database under NM_005018.2 and NP_005009.2 and is shown in Table 1 (see also Ishida et al. (1992) 20 EMBO J 11:3887; Shinohara et al. (1994) Genomics 23:704; U.S. Pat. No. 5,698,520). PD-1 has an extracellular region containing immunoglobulin superfamily domain, a transmembrane domain, and an intracellular region including an immunoreceptor tyrosine-based inhibitory motif (ITIM) (Ishida et al. (1992) EMBO J. 11:3887; Shinohara et al. (1994) Genomics 23:704; and U.S. Pat. No. 5,698,520) and an immunoreceptor tyrosine-based switch motif (ITSM). These features also define a larger family of polypeptides, called the immunoinhibitory receptors, which also includes gp49B, PIR-B, and the killer inhibitory receptors (KIRs) (Vivier and Daeron (1997) Immunol. Today 18:286). It is often assumed that the tyrosyl phosphorylated ITIM and ITSM motif of these receptors interacts with SH2-domain containing phosphatases, which leads to inhibitory signals. A subset of these immunoinhibitory receptors bind to MHC polypeptides, for example the KIRs, and CTLA4 binds to B7-1 and B7-2. It has been proposed that there is a phylogenetic relationship between the MHC and B7 genes (Henry et al. (1999) Immunol. Today 20(6):285-8).

The term “response to immunotherapy” or “response to inhibitor(s) of one or more biomarkers listed in Table 1, in combination with an immunotherapy” relates to any response of the hyperproliferative disorder (e.g., cancer) to an anti-cancer agent, such as an inhibitor of one or more biomarkers listed in Table 1, and an immunotherapy, preferably to a change in tumor mass and/or volume after initiation of neoadjuvant or adjuvant therapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for which biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 25% or more, for example, 30%, 40%, 50%, 60%, 70%, 80%, or more, to 2-fold, 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

As used herein, the term “unresponsiveness” includes refractivity of cancer cells to therapy or refractivity of therapeutic cells, such as immune cells, to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134).

### NK Cells

Natural killer cells or NK cells are a type of cytotoxic lymphocyte critical to the innate immune system. The role NK cells play is analogous to that of cytotoxic T cells in the vertebrate adaptive immune response. NK cells provide rapid responses to viral-infected cells, acting at around 3 days after infection, and respond to tumor formation. Typically, immune cells detect major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. They were named “natural killers” because of the initial notion that they do not require activation to kill cells that are missing “self” markers of MHC class 1. This role is especially important because harmful cells that are missing MHC I markers cannot be detected and destroyed by other immune cells, such as T lymphocyte cells.

NK cells (belonging to the group of innate lymphoid cells) are defined as large granular lymphocytes (LGL) and constitute the third kind of cells differentiated from the common lymphoid progenitor-generating B and T lymphocytes. NK cells are known to differentiate and mature in the bone marrow, lymph nodes, spleen, tonsils, and thymus, where they then enter into the circulation. NK cells differ from natural killer T cells (NKTs) phenotypically, by origin and by respective effector functions; often, NKT cell activity promotes NK cell activity by secreting IFNγ. In contrast to NKT cells, NK cells do not express T-cell antigen receptors (TCR) or pan T marker CD3 or surface immunoglobulins (Ig) B cell receptors, but they usually express the surface markers CD16 (FcγRIII) and CD56 in humans, NK1.1 or NK1.2 in C57BL/6 mice. The NKp46 cell surface marker constitutes, at the moment, another NK cell marker of preference being expressed in both humans, several strains of mice (including BALB/c mice) and in three common monkey species.

NK cells are negatively regulated by major histocompatibility complex (MHC) class I-specific inhibitory receptors (Karre et al., 1986; Ohlen et al, 1989). These specific receptors bind to polymorphic determinants of MHC class I molecules or HLA present on other cells and inhibit NK cell lysis. In humans, certain members of a family of receptors termed killer Ig-like receptors (KIRs) recognize groups of HLA class I alleles.

KIRs are a large family of receptors present on certain subsets of lymphocytes, including NK cells. The nomenclature for KIRs is based upon the number of extracellular domains (KIR2D or KIR3D) and whether the cytoplasmic tail is either long (KIR2DL or KIR3DL) or short (KIR2DS or KIR3DS). Within humans, the presence or absence of a given KIR is variable from one NK cell to another within the NK population present in a single individual. Within the human population there is also a relatively high level of polymorphism of the KIR molecules, with certain KIR molecules being present in some, but not all individuals. Certain KIR gene products cause stimulation of lymphocyte activity when bound to an appropriate ligand. The confirmed stimulatory KIRs all have a short cytoplasmic tail with a charged transmembrane residue that associates with an adapter molecule having an immunostimulatory motif (ITAM). Other KIR gene products are inhibitory in nature.

Natural killer cells constitute about 10% of peripheral blood mononuclear cells in human blood, and are identified by their lack of surface expression of CD3 and expressions of CD16 and CD56. NK cells mediate both direct and antibody-dependent cellular cytotoxicity (ADCC) against tumor cells and virally infected cells. They can recognize these cells without prior sensitization. NK cells mediate direct cytotoxicity by releasing pre-formed granules known as perforin and granzyme B, which can induce necrosis and apoptosis. When NK cell recognize its target cells and forms the lytic immunological synapse, the secretory lysosome polarizes towards the synapse and move into close proximity to the plasma membrane. Perforin, a membrane-disrupting protein, facilitates delivery of the Granzyme, a serine protease, which cleaves a variety of targets, such as caspases, resulting in cell death. NK cells can also mediate direct cytotoxicity via death receptors on the target cells through surface expression of their ligands such as Fas Ligand, Trail and TNF-alpha. Fas (CD95/AP0-1/TNFRSF6), a cell surface protein that belongs to the tumor necrosis factor receptor family, can mediate apoptosis when bound to its natural ligand, CD95L (CD178/TNFSF6) or stimulated with agonistic antibodies. NK cells can mediate antibody dependent cellular cytotoxicity (ADCC) against tumors and regulate the function of other cells through the secretion of cytokines and chemokines.

Two major subsets of NK cells have been identified, one with the surface expression of CD16⁺⁺⁺CD56+, which is the predominant subset in the circulating blood with high cytotoxicity, whereas the other is CD16⁻CD56⁺⁺⁺ subset residing in the mucosa known as the regulatory subset. Our Laboratory has established four different stages of NK cell maturation. Stage one NK cells are CD16⁺⁺⁺, CD56⁺, CD69⁻, and CD107a⁻ found to select and kill cancer stem-like cells/undifferentiated tumors. Upon IL-2 activation and CD16 receptor triggering NK cells express CD16^{+/-}CD56⁺⁺CD69⁺CD107a⁺ and increase secretion of IFN-γ and TNF-α while exhibiting decreased cytotoxicity. This is the second stage and NK cells in this stage are known as split-anergized NK cells. Without further activation NK cells move towards stage three where they become non-functional and lose their cytotoxicity and cytokine secretion ability. Finally, NK cells may undergo apoptosis giving rise to stage 4.

### Probiotic Bacteria

In some embodiments, the instant invention is drawn to a composition comprising at least one probiotic bacterial strain, capable of regulating NK cell function. Such probiotic bacteria induce significant split anergy in activated NK cells, leading to a significant induction of IFN-γ and TNF-α. In addition, such probiotic bacteria induce significant expansion of NK cells. Exemplary probiotic bacteria useful for this purpose are disclosed in International Patent Application WO18/112366, hereby incorporated herein by reference, in particular for the probiotic bacteria it discloses.

Many commercial probiotics are available, having various effects of reducting gastrointestinal discomfort or strengthening of the immune system. Preferred probiotic bacteria species for use in the compositions and methods described herein include those commercially available strains of probiotic bacteria (such as AJ2 bacteria), especially those from the Streptococcus (e.g., S. thermophiles), Bifidobacterium (e.g., B. longum, B. breve, B. infantis, B. breve, B. infantis), and Lactobacillus genera (e.g., L. acidophilus, L. helveticus, L. bulgaricus, L. rhamnosus, L. plantarum, and L. casei). The instant disclosure comprising methods of administering at least one probiotic bacterial strain, preferably a combination of two or more different bacterial strains, to a subject, preferably a mammal (e.g., a human). Such administration may be systemically or locally (e.g., directly to intestines) performed. A preferably administration route is oral administration. Other routes (e.g., rectal) may be also used. For administration, either the bacteria (e.g., in a wet, sonicated, grounded, or dried form or formula), the bacterial culture medium containing the bacteria, or the bacterial culture medium supernatant (not containing the bacteria), may be administered.

AJ2 is a combination of eight strains of gram positive probiotic bacteria with the ability to induce synergistic production of IFN-γ when added to IL-2-treated or IL-2+anti-CD16 monoclonal antibody-treated NK cells (anti-CD16mAb). The combination of strains was used to provide bacterial diversity in addition to synergistic induction of a balanced pro and anti-inflammatory cytokine and growth factor release NK cells. Moreover, the quantity of each bacteria within the combination of strains was adjusted to yield a closer ratio of IFN-γ to IL-10 to that obtained when NK cells are activated with IL-2+anti-CD16mAb in the absence of bacteria. The rationale behind such selection was to obtain a ratio similar to that obtained with NK cells activated with IL-2+anti-CD16mAb in the absence of bacteria since such treatment provided significant differentiation of the cells.

### Antibody-Dependent Cell-Cytotoxicity

Antibody-dependent cellular-cytotoxicity (ADCC), is a mechanism by which immune cells bearing the Fc receptor can kill the cells coated with the antibody upon binding of the Fc receptor to the Fc portion of the antibody. NK cells are one the subset of immune cells that can mediate ADCC through FcγRIIIA receptor also known as CD16. The mechanism by which NK cells mediate ADCC is not fully understood. When the effector cell recognizes the target by cross-linking of the Fc receptor and the antibody coating the target cell, the immunoreceptor tyrosine-based activation motifs (ITAMs) gets phosphorylated in the effector cells and leading to triggering of main downstream signaling pathways in the effector cell to kill the target cell. One of the mechanisms by which NK cells mediated ADCC can be through perforin-granzyme mediate cytotoxicity. The role of FAS ligand in ADCC is unknown but It has been shown that cross-linking of the CD16 receptor on NK cells can upregulate FAS ligand on them which may be indicative an important role of Fas/Fas-L in ADCC.

### Fucoidan

Fucoidan is a sulfated polysaccharide found on different species of brown algae and brown seaweed such as Mozuko, Mekabue, Limi moui, bladderwrack, and hijiki. Based on the source of extraction, Fucoidan may have different chemical compositions. For example, beside the polysaccharide and the sulfate, they might also contain other monosaccharides (mannose, galactose, glucose, xylose, etc.) uranic acids, acetyl groups, and protein. Undaria pinnatifida, also known as Mekabue seaweed is another source of Fucoidan composing fucose, galactose, and sulfate.

### Split Anergy

Split anergy is a maturation stage of NK cells, wherein NK cells show reduced cytotoxicity andaugmented secretion of IFN-γ. Split-anergized NK cells promote differentiation of target cells via secreted and membrane-bound factors, increase tumor cell resistance to NK cell-mediated cytotoxicity, as well as inhibit inflammation due to the reduction of cytokine and chemokine production after tumor differentiation.

### Cancer Stem Cells

Cancer stem cells (CSCs) are stem cells which can create various populations of differentiated cells that define the tumor mass. CSCs are like normal stem cells, and have self-renewal capacity and also can be differentiated, but in a dysregulated manner. The existence of CSCs is described in many tumors including, but not limited to, acute myeloid leukemia, breast, prostate, melanoma, lung, colon, brain, liver, gastric and pancreatic cancer.

### Osteoclasts

Osteoclast are the bone cells responsible for the bone homeostasis by resorbing the bone. Osteoclast matures via RANKL stimulation and the process is regulated by ICAM-1. Proinflammatory signals can induce expression of ICAM-1 and RANKL on osteoclasts. These signals are mediated by subsets of immune cells. It has been shown that osteoclasts express multiple ligands for both activating and inhibitory NK cell receptors.

### MICA/MICB

Major Histocompatibility Complex Class I-Related Chains A and B (MICA/MICB) are proteins known to be induced upon stress, damage, viral infection or transformation of cells which act as a ‘kill me’ signal through the cytotoxic lymphocytes. In contrast to classical MHC class-I molecules, this protein is not involved in antigen presenting but they are known to be a ligand for a natural killer group 2D (NKG2D) receptor, a receptor on cytotoxic cells. Engagement of NKG2D receptors triggers natural killer (NK) cell-mediated cytotoxicity and provides a costimulatory signal for CD8 T cells and γδ T cells. MICAS were not thought to be constitutively expressed by healthy normal cells, but recently studies have shown that this protein is also expressed on surface of healthy cells such as breast, colon, liver, pancreas, stomach, bronchus, bladder and ureter in smooth muscle cells and/or myofibroblasts within stomach, small intestine, colon, bladder, cervix, fallopian tube, prostate and ureter. The differential expression of MICA/MICB based on the differentiation status of the tumor cells have not be studied. In this study, we will evaluate the expression of MICA/MICB on the undifferentiated/stem-like and differentiated oral and pancreatic tumors.

### II. Subjects

In certain embodiments, the subject suitable for the compositions and methods disclosed herein is a mammal (e.g., mouse, rat, primate, non-human mammal, domestic animal, such as a dog, cat, cow, horse, and the like), and is preferably a human.

In certain embodiments, the subject is an animal model of cancer. For example, the animal model can be an orthotopic xenograft animal model of a human-derived cancer.

In various embodiments of the methods of the present invention, the subject has not undergone treatment, such as chemotherapy, radiation therapy, targeted therapy, and/or immunotherapies. In other embodiments, the subject has undergone treatment, such as chemotherapy, radiation therapy, targeted therapy, and/or immunotherapies.

In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

### III. Anti-Cancer Therapies

In one aspect, other anti-cancer therapies and/or immunotherapies combination or combinations of therapies (e.g., one or more PI3Kbeta-selective inhibitors, such as KIN193, in combination with one or more immune checkpoint inhibitors, such as an anti-PD-1 antibody, either alone or in combination with yet additional anti-cancer therapies, such as targeted therapy) can be administered (e.g., conjointly).

The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in separate formulations, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

Combination therapies are also contemplated and can comprise, for example, one or more chemotherapeutic agents and radiation, one or more chemotherapeutic agents and immunotherapy, or one or more chemotherapeutic agents, radiation and chemotherapy, each combination of which can be with a therapy as disclosed herein. As described below, agents can be administered in combination therapy with, e.g., chemotherapeutic agents, hormones, antiangiogens, radiolabelled compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, these modulatory agents can be administered with a therapeutically effective dose of chemotherapeutic agent. In other embodiments, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular melanoma, being treated, the extent of the disease and other factors familiar to the physician of skill in the art and can be determined by the physician.

The term “targeted therapy” refers to administration of agents that selectively interact with a chosen biomolecule to thereby treat cancer. One example includes immunotherapies such as immune checkpoint inhibitors, which are well-known in the art. For example, anti-PD-1 pathway agents, such as therapeutic monoclonal blocking antibodies, which are well-known in the art and described above, can be used to target tumor microenvironments and cells expressing unwanted components of the PD-1 pathway, such as PD-1, PD-L1, and/or PD-L2.

For example, the term “PD-1 pathway” refers to the PD-1 receptor and its ligands, PD-L1 and PD-L2. “PD-1 pathway inhibitors” block or otherwise reduce the interaction between PD-1 and one or both of its ligands such that the immunoinhibitory signaling otherwise generated by the interaction is blocked or otherwise reduced. Anti-immune checkpoint inhibitors can be direct or indirect. Direct anti-immune checkpoint inhibitors block or otherwise reduce the interaction between an immune checkpoint and at least one of its ligands. For example, PD-1 inhibitors can block PD-1 binding with one or both of its ligands. Direct PD-1 combination inhibitors are well-known in the art, especially since the natural binding partners of PD-1 (e.g., PD-L1 and PD-L2), PD-L1 (e.g., PD-1 and B7-1), and PD-L2 (e.g., PD-1 and RGMb) are known.

For example, agents which directly block the interaction between PD-1 and PD-L1, PD-1 and PD-L2, PD-1 and both PD-L1 and PD-L2, such as a bispecific antibody, can prevent inhibitory signaling and upregulate an immune response (i.e., as a PD-1 pathway inhibitor). Alternatively, agents that indirectly block the interaction between PD-1 and one or both of its ligands can prevent inhibitory signaling and upregulate an immune response. For example, B7-1 or a soluble form thereof, by binding to a PD-L1 polypeptide indirectly reduces the effective concentration of PD-L1 polypeptide available to bind to PD-1. Exemplary agents include monospecific or bispecific blocking antibodies against PD-1, PD-L1, and/or PD-L2 that block the interaction between the receptor and ligand(s); a non-activating form of PD-1, PD-L1, and/or PD-L2 (e.g., a dominant negative or soluble polypeptide), small molecules or peptides that block the interaction between PD-1, PD-L1, and/or PD-L2; fusion proteins (e.g. the extracellular portion of PD-1, PD-L1, and/or PD-L2, fused to the Fc portion of an antibody or immunoglobulin) that bind to PD-1, PD-L1, and/or PD-L2 and inhibit the interaction between the receptor and ligand(s); a non-activating form of a natural PD-1, PD-L2, and/or PD-L2 ligand, and a soluble form of a natural PD-1, PD-L2, and/or PD-L2 ligand.

Indirect anti-immune checkpoint inhibitors block or otherwise reduce the immunoinhibitory signaling generated by the interaction between the immune checkpoint and at least one of its ligands. For example, an inhibitor can block the interaction between PD-1 and one or both of its ligands without necessarily directly blocking the interaction between PD-1 and one or both of its ligands. For example, indirect inhibitors include intrabodies that bind the intracellular portion of PD-1 and/or PD-L1 required to signal to block or otherwise reduce the immunoinhibitory signaling. Similarly, nucleic acids that reduce the expression of PD-1, PD-L1, and/or PD-L2 can indirectly inhibit the interaction between PD-1 and one or both of its ligands by removing the availability of components for interaction. Such nucleic acid molecules can block PD-L1, PD-L2, and/or PD-L2 transcription or translation.

Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In certain embodiments, immunotherapy comprises adoptive cell-based immunotherapies. Well-known adoptive cell-based immunotherapeutic modalities, including, without limitation, Irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (MET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, patient-specific neoantigen vaccines, and the like.

In other embodiments, immunotherapy comprises non-cell-based immunotherapies. In certain such embodiments, compositions comprising antigens with or without vaccine-enhancing adjuvants are used. Such compositions exist in many well-known forms, such as peptide compositions, oncolytic viruses, recombinant antigen comprising fusion proteins, and the like. In still other embodiments, immunomodulatory interleukins, such as IL-2, IL-6, IL-7, IL-12, IL-17, IL-23, and the like, as well as modulators thereof (e.g., blocking antibodies or more potent or longer lasting forms) are used. In yet other embodiments, immunomodulatory cytokines, such as interferons, G-CSF, imiquimod, TNFalpha, and the like, as well as modulators thereof (e.g., blocking antibodies or more potent or longer lasting forms) are used. In other embodiments, immunomodulatory chemokines, such as CCL3, CCL26, and CXCL7, and the like, as well as modulators thereof (e.g., blocking antibodies or more potent or longer lasting forms) are used. In other embodiments, immunomodulatory molecules targeting immunosuppression, such as STAT3 signaling modulators, NFkappaB signaling modulators, and immune checkpoint modulators, are used. The terms “immune checkpoint” and “anti-immune checkpoint therapy” are described above.

In still other embodiments, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C (indole-3-carbinol)/DIM (di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet other embodiments, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, agents and therapies other than immunotherapy or in combination thereof can be used with in combination with inhibitors of one or more biomarkers listed in Table 1, with or without immunotherapies to stimulate an immune response to thereby treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

Alternatively, immunotherapy may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In certain embodiments, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In certain embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1, 8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In other embodiments, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In yet other embodiments, surgical intervention can physically remove cancerous cells and/or tissues.

In still other embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In yet other embodiments, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still other embodiments, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells.

In yet other embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. The laser light then raises the temperature of the tumor, which damages or destroys cancer cells.

The duration and/or dose of treatment with therapies may vary according to the particular therapeutic agent or combination thereof. An appropriate treatment time for a particular cancer therapeutic agent will be appreciated by the skilled artisan. The present invention contemplates the continued assessment of optimal treatment schedules for each cancer therapeutic agent, where the phenotype of the cancer of the subject as determined by the methods of the present invention is a factor in determining optimal treatment doses and schedules.

In other embodiments, recombinant biomarker polypeptides, and fragments thereof, can be administered to subjects. In some embodiments, fusion proteins can be constructed and administered which have enhanced biological properties. In addition, the biomarker polypeptides, and fragment thereof, can be modified according to well-known pharmacological methods in the art (e.g., pegylation, glycosylation, oligomerization, etc.) in order to further enhance desirable biological activities, such as increased bioavailability and decreased proteolytic degradation.

### IV. Treatment Methods

The therapeutic compositions described herein can be used in a variety of in vitro and in vivo therapeutic applications using the formulations and/or combinations described herein. In various embodiments, the therapeutic agents can be used to treat cancers determined to be responsive thereto.

Modulatory methods of the present invention involve contacting a cell, such as an immune cell with an agent that inhibits or blocks the expression and/or activity of such one or more biomarkers and an immunotherapy, such as an immune checkpoint inhibitor (e.g., PD-1). Exemplary agents useful in such methods are described above. Such agents can be administered in vitro or ex vivo (e.g., by contacting the cell with the agent) or, alternatively, in vivo (e.g., by administering the agent to a subject). As such, the present invention provides methods useful for treating an individual afflicted with a condition that would benefit from an increased immune response, such as an infection or a cancer like colorectal cancer.

Agents that upregulate immune responses can be in the form of enhancing an existing immune response or eliciting an initial immune response. Thus, enhancing an immune response using the subject compositions and methods is useful for treating cancer, but can also be useful for treating an infectious disease (e.g., bacteria, viruses, or parasites), a parasitic infection, and an immunosuppressive disease.

Exemplary infectious disorders include viral skin diseases, such as Herpes or shingles, in which case such an agent can be delivered topically to the skin. In addition, systemic viral diseases, such as influenza, the common cold, and encephalitis might be alleviated by systemic administration of such agents. For example, agents that upregulate the immune response described herein are useful for modulating the arginase/iNOS balance during Trypanosoma cruzi infection in order to facilitate a protective immune response against the parasite.

Immune responses can also be enhanced in an infected patient through an ex vivo approach, for instance, by removing immune cells from the patient, contacting immune cells in vitro with an agent described herein and reintroducing the in vitro stimulated immune cells into the patient.

In certain instances, it may be desirable to further administer other agents that upregulate immune responses, for example, forms of other B7 family members that transduce signals via costimulatory receptors, in order to further augment the immune response. Such additional agents and therapies are described further below.

Agents that upregulate an immune response can be used prophylactically in vaccines against various polypeptides (e.g., polypeptides derived from pathogens). Immunity against a pathogen (e.g., a virus) can be induced by vaccinating with a viral protein along with an agent that upregulates an immune response, in an appropriate adjuvant.

Additionally, upregulation or enhancement of an immune response function, as described herein, is useful in the induction of tumor immunity.

Furthermore, the immune response can be stimulated by the methods described herein, such that preexisting tolerance, clonal deletion, and/or exhaustion (e.g., T cell exhaustion) is overcome. For example, immune responses against antigens to which a subject cannot mount a significant immune response, e.g., to an autologous antigen, such as a tumor specific antigens can be induced by administering appropriate agents described herein that upregulate the immune response. Similarly, an autologous antigen, such as a tumor-specific antigen, can be coadministered. In addition, the subject compositions can be used as adjuvants to boost responses to foreign antigens in the process of active immunization.

In certain embodiments, immune cells are obtained from a subject and cultured ex vivo in the presence of an agent as described herein, to expand the population of immune cells and/or to enhance immune cell activation. The immune cells may then be administered to a subject. Immune cells can be stimulated in vitro by, for example, providing to the immune cells a primary activation signal and a costimulatory signal, as is known in the art. Various agents can also be used to costimulate proliferation of immune cells. Immune cells may be cultured ex vivo according to the method described in PCT Application No. WO 94/29436. The costimulatory polypeptide can be soluble, attached to a cell membrane, or attached to a solid surface, such as a bead.

### V. Administration of Agents

The immune modulating agents of the invention are administered to subjects in a biologically compatible form suitable for pharmaceutical administration in vivo, to enhance immune cell mediated immune responses. By “biologically compatible form suitable for administration in vivo” is meant a form to be administered in which any toxic effects are outweighed by the therapeutic effects. The term “subject” is intended to include living organisms in which an immune response can be elicited, e.g., mammals. Examples of subjects include humans, dogs, cats, mice, rats, and transgenic species thereof. Administration of an agent as described herein can be in any pharmacological form including a therapeutically active amount of an agent alone or in combination with a pharmaceutically acceptable carrier.

Administration of a therapeutically active amount of the therapeutic composition of the present invention is defined as an amount effective, at dosages and for periods of time necessary, to achieve the desired result. For example, a therapeutically active amount of an agent may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

The therapeutic agents described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well-known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Pharmaceutical compositions of this invention suitable for parenteral administration comprise one or more therapeutic agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the therapeutic agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

In certain embodiments, a component of the compositions of the invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays.

The therapeutic compositions of the present invention may also include known antioxidants, buffering agents, and other agents such as coloring agents, flavorings, vitamins or minerals.

### EXAMPLES

### Example 1: Materials and Methods for Examples 2-10

### Cell Lines, Reagents, and Antibodies

RPMI 1640 supplemented with 10% fetal bovine serum (FBS) (Gemini Bio-Products, CA, USA) was used for the cultures of human NK cells and monocytes. OSCCs and stem-like OSCSCs were isolated from oral cancer patient tongue tumors at UCLA, and cultured in RPMI 1640 supplemented with 10% FBS (Gemini Bio-Products, CA, USA), 1.4% antibiotic antimycotic, 1% sodium pyruvate, 1.4% non-essential amino acids, 1% L-glutamine, 0.2% gentamicin (Gemini Bio-Products, CA, USA), and 0.15% sodium bicarbonate (Fisher Scientific, PA, USA). Mia-Paca-2 (MP2) were cultured in DMEM with 10% FBS and 1% penicillin and streptomycin (Gemini Bio-Products, CA, USA). Recombinant IL-2 was obtained from NIH-BRB. Recombinant TNF-α and IFN-γ were obtained from BioLegend (San Diego, Calif., USA). Anti-MHC class I was prepared and 1:100 dilution was found to be the optimal concentration to use. PE conjugated anti-CD54, anti-CD44, anti-B7H1, anti-MICA/MICB antibody were obtained from BioLegend (San Diego, Calif., USA). Antibody against MICA/MICB was a generous gift from Dr. Jennifer Wu from Feinberg school of medicine. The human NK and monocyte purification kits were obtained from Stem Cell Technologies (Vancouver, BC, Canada).

Purification of NK Cells and T Cells from the Human Peripheral Blood

Written informed consents, approved by UCLA Institutional Review Board (IRB), were obtained from healthy blood donors, and all procedures were approved by the UCLA-IRB. Peripheral blood was separated using Ficoll-Hypaque centrifugation, after which the white, cloudy layer, containing peripheral blood mononuclear cells (PBMC), was harvested, washed and resuspended in RPMI 1640 (Invitrogen by Life Technologies, CA) supplemented with 10% FBS and plated on plastic tissue culture dishes. After 1-2 hours of incubation, non-adherent, human peripheral blood lymphocytes (PBL) were collected. NK cells were negatively selected and isolated from PBLs using the EasySep® Human NK cell enrichment kit and T cells isolation kit, respectively purchased from Stem Cell Technologies (Vancouver, BC, Canada). Isolated NK cells were stained with anti-CD16 and anti-CD3 antibody, respectively, to measure the cell purity using flow cytometric analysis. Purified NK cells were cultured in RPMI Medium 1640 supplemented with 10% FBS (Gemini Bio-Products, CA), 1% antibiotic/antimycotic, 1% sodium pyruvate, and 1% MEM non-essential amino acids (Invitrogen, Life Technologies, CA).

### Expansion of NK Cells

Human purified and hu-BLT enriched NK cells were activated with rh-IL-2 (1000 U/ml) and anti-CD16mAb (3 ug/ml) for 18-20 hours before they were co-cultured with feeder cells and sAJ2. The culture media was refreshed with rh-IL-2 every three days [43].

### NK Cells Supernatants Used for Stem Cell Differentiation

As described above, human NK cells were purified from PBMCs of healthy donors. NK cells were treated with a combination of anti-CD16mAb (3 μg/mL) and IL-2 (1,000 U/mL) for 18 hours before supernatants were removed and used for differentiation experiments. The amounts of IFN-γ produced by activated NK cells were assess with IFN-γ ELISA (BioLegend, CA, USA). OSCSCs were differentiated with gradual daily addition of increasing amounts of NK cell supernatants. On average, to induce differentiation, a total of 3,500 pg. of IFN-γ containing supernatants were added for 5 days to induce differentiation and resistance of OSCSCSs to NK cell-mediated cytotoxicity and a total of 7000 pg. of IFN-γ containing supernatants were added for 7 days to induce differentiation and resistance of MP2 to NK cell-mediated cytotoxicity. Afterwards, target cells were washed with PBS, detached and used for experiments.

Treating NK Cell with Mekabue

The Fucoidan extracted from the Mekabue seaweed was purchased from NatureMedic. 12.5 g of the Mekabue extracted fucoidan (Mekabue) was solubilized in 1 ml of PBS.1 and then added to cultures.

### Sonicating AJ2

AJ2 was weighed and resuspended in RPMI Medium 1640 containing 10% FBS at a concentration of 10 mg/mL. The bacteria were thoroughly vortexed, then sonicated on ice for 15 seconds, at 6 to 8 amplitudes. Sonicated samples were then incubated for 30 seconds on ice. After every five pulses, a sample was taken to observe under the microscope until at least 80 percent of cell walls were lysed. It was determined that approximated 20 rounds of sonication/incubation on ice, were conducted to achieve complete sonication. Finally, the sonicated samples (sAJ2) were aliquoted and stored in a −80° C. freezer.

### Generation of Osteoclasts

Osteoclasts were generated from PBMC-purified monocytes and cultured in alpha-MEM medium, containing M-CSF (25 ng/mL) and RANK Ligand (RANKL) (25 ng/mL), for 21 days. 14 Medium was refreshed every 3 days with fresh alpha-MEM, containing M-CSF (25 ng/mL) and RANKL (25 ng/mL).

### ADCC Induction

The target cells (5×10⁵) were labeled with 50 μCi ⁵¹Cr (Perkin Elmer, Santa Clara, Calif.) and chromated for 1 hour. Following incubation, target cells were washed once to remove excess unbound ⁵¹Cr. Cells were resuspended in 1×10⁶/mL and the treated with the anti-MICA/MICB antibody or Cetaximab (3 μg/mL) and incubated for 30 minutes. Following incubation, target cells were washed again to remove excess unbound antibody and ⁵¹Cr. Labeled target cells were culture with effector cells and the cytotoxicity against target cells were assessed using ⁵¹Cr release cytotoxicity assay.

⁵¹Cr Release Cytotoxicity Assay ⁵¹Cr was purchased from Perkin Elmer (Santa Clara, Calif.). Standard ⁵¹Cr release cytotoxicity assays were used to determine NK cell cytotoxic function in the experimental cultures. The effector cells (1×10⁵ cells/well) were aliquoted into 96-well round-bottom micro-well plates (Fisher Scientific, Pittsburgh, Pa.) and titrated at 4 to 8 serial dilutions. Target cells (5×10⁵) were labeled with 50 μCi ⁵¹Cr (Perkin Elmer, Santa Clara, Calif.) and chromated for 1 hour. Following incubation, target cells were washed twice to remove excess unbound ⁵¹Cr. ⁵¹Cr-labeled target cells were aliquoted into the 96-well round bottom microwell plates containing effector cells at a concentration of 1×10⁴ cells/well at a top effector:target (E:T) ratio of 5:1. Plates were centrifuged and incubated for a period of 4 hours. After a 4-hour incubation period, the supernatants were harvested from each sample and counted for released radioactivity using the gamma counter. Total (containing ⁵¹Cr labeled target cells) and spontaneous (supernatants of target cells alone) release values were measured and used to calculate the percentage specific cytotoxicity. The percentage specific cytotoxicity was calculated using the following formula:

% Cytotoxicity=[Experimental cpm−spontaneous cpm]/[Total cpm−spontaneous cpm]

Lu30/10⁶ is calculated by using the inverse of the number of effector cells needed to lyse 30% of target cells×100.

### Enzyme-Linked Immunosorbent Assays (ELISAs)

ELISA kit for IFN-γ was purchased from BioLegend (San Diego, Calif.). ELISA was performed to detect the level of IFN-γ produced from cell cultures. The assay was conducted as described in the manufacturer's protocol. Briefly, 96-well EIA/RIA plates were coated with diluted capture antibody corresponding to target cytokine and incubated overnight at 4° C. After 16-18 hours of incubation, the plates were washed 4 times with wash 18 buffer (0.05% Tween in 1×PBS) and blocked with assay diluent (1% BSA in 1×PBS). The plates were incubated for 1 hour at room temperature, on a plate shaker at 200 rpm; plates were washed 4 times following incubation. Then, 100 μL of standards and samples collected from each culture were added to the wells and incubated for 2 hours at room temperature, on the plate shaker at 200 rpm. After incubation, plates were washed 4 times, loaded with detection antibody, and incubated for 1 hour at room temperature, on the plate shaker at 200 rpm. After 1 hour of incubation, the plates were washed 4 times; wells were loaded with Avidin-HRP solution and incubated for 30 minutes at room temperature, on the plate shaker at 200 rpm. After washing the plates 5 times with wash buffer; 100 uL of TMB substrate solution was added to the wells and plates were incubated in the dark until they developed a desired blue color (or up to 30 minutes). Then, 100 μL of stop solution (2N H₂SO₄) was added per well to stop the reaction. Finally, plates were read in a microplate reader, at 450 nm to obtain absorbance values (BioLegend, ELISA manual).

### Surface Staining

1×10⁵ cells from each condition were stained in 100 μL of cold 1% BSA-PBS with predetermined optimal concentration of PE conjugated antibodies, as detailed in the experiments, and incubated at 4° C. for 30 minutes. Then, cells were washed and resuspended in 1% BSA-PBS. The Epics C (Coulter) flow cytometer was used for cellular surface analysis.

### Statistical Analysis

An unpaired or paired two-tailed Student's t-test were performed to compare different groups depending on the experimental design. The p-values were expressed within the figures as follows: ***p-value<0.001, **p-value: 0.001-0.01, *p-value: 0.01-0.05. The GraphPad Prism software was used to analyze the data.

### Example 2: NK Cells Mediate Higher Cytotoxicity Against Undifferentiated/Stem-Like Oral and Pancreatic Tumor Cells in Comparison to their Differentiated Compartments

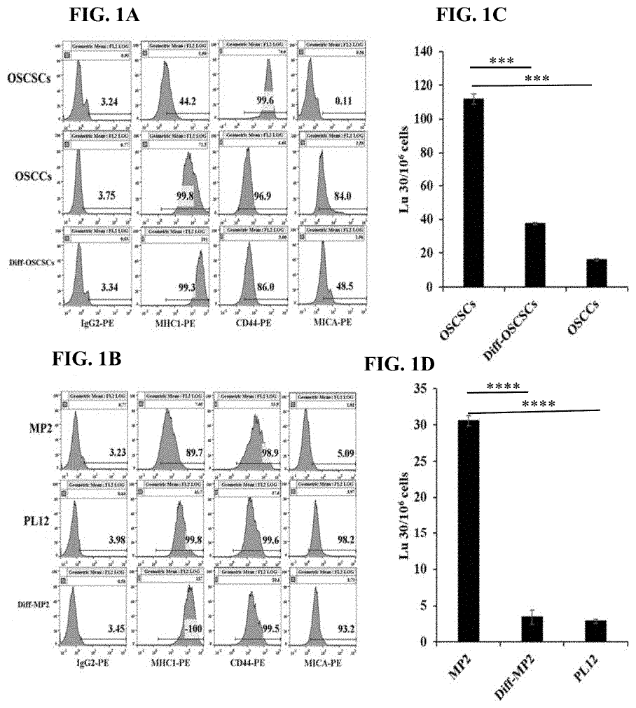

OSCSCs and MP2 displayed higher expression of CD44, and lower expression of MHC-I, MICA and CD54, while the reverse profile was seen in their differentiated compartments. To differentiate the OSCSCs, and MP2 the tumor cells were treated with supernatants from split-anergized NK cells as described in Example 1. Treatment of OSCSCs and MP2 with split-anergized NK cells supernatant decreased the CD44 surface expression and increased the MHC-I, MICA and CD54 surface expression (FIGS. 1A and 1B). NK cells mediated much higher cytotoxicity against oral squamous carcinoma stem-like cells (OSCSCs), and undifferentiated/stem-like pancreatic tumor cells (MP2), when compared to the differentiated oral squamous carcinoma cells (OSCCs) and pancreatic tumor cells (PL12). (FIGS. 1C and 1D). The treatment of OSCSCs with split-anergized NK cell supernatants significantly decreased their sensitivity to IL-2-treated NK cell-mediated lysis.

### Example 3: Differentiated Pancreatic and Oral Tumors Expressed Higher Level of MICA/MICB in Comparison to their Undifferentiated Compartments

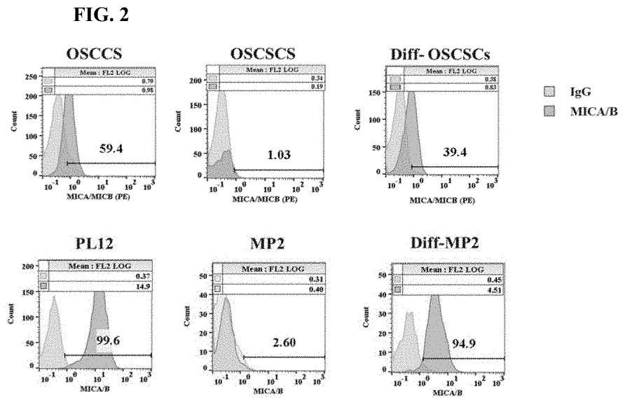

OSCSCs and MP2 displayed lower expression of MICA/MICB, while the reverse profile was seen in their differentiated compartments, OSCCs and PL12. Treatment of OSCSCs and MP2 with split-anergized NK cells supernatant increase the MICA/MICB surface expression showing that well-differentiated tumors express higher level of MICA/MICB in comparison to undifferentiated/stem-like oral and pancreatic tumors (FIG. 2).

### Example 4: Antibodies Specific to MICA/MICB Increased NK Cell-Mediated ADCC Against OSCC, while OSCSCs were not Targeted

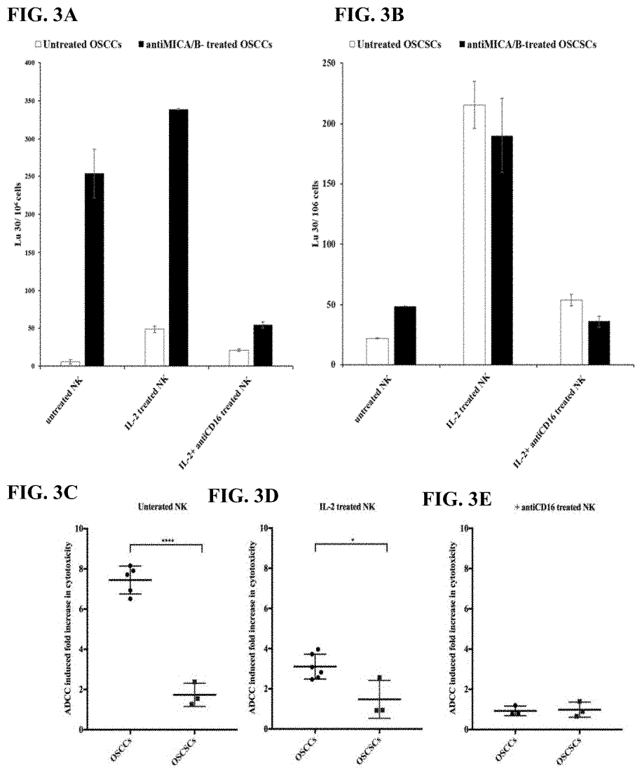

To study the antibody dependent mediated cytotoxicity (ADCC) in NK cells, against differentiated tumor cells expressing high level of MICA/MICB and their undifferentiated compartments expressing low level of MICA/MICB, NK cells were purified from healthy donors. NK cells were left untreated, treated with IL-2, or the combination of IL-2 and anti-CD16mAb. Their cytotoxicity against the OSCSCs and OSCCs untreated or treated with the antibody against MICA/MICB was determined using the ⁵¹Cr release assay. Untreated and IL-2 treated NK cells mediated higher cytotoxicity against anti-MICAS treated OSCCS in comparison to untreated OSCCS (FIG. 3A). The fold increase in cytotoxicity against antiMICA/MICB treated OSCCs was significantly higher in untreated (mean=6.9 fold increase) and IL-2 treated NK (mean=3.1 fold increase) than untreated OSCCs (FIGS. 3C and 3D), while the differences in cytotoxicity of untreated and IL-2 treated NK cells against untreated and anti-MICAS treated OSCSCS were not significantly different (FIGS. 3B-3D). NK cells treated with combination of IL-2 and anti-CD16mAb did not mediate significant levels of ADCC against OSCSCs and OSCCS. (FIGS. 3A-3D).

### Example 5: Antibodies Specific to MICA/MICB Increased NK Cell-Mediated ADCC Against PL12, while MP2 Cells were not Targeted

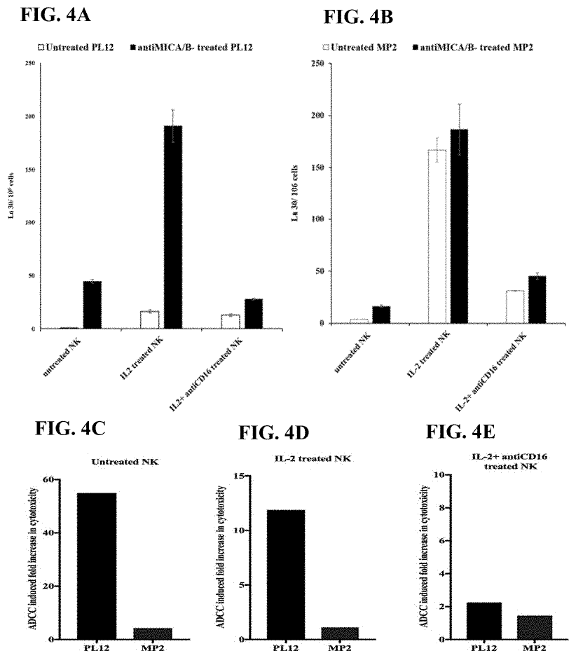

To further confirm that the same observation can be seen in pancreatic tumor, the same experiment in Example 4 was conducted with MP2 and PL12. The cytotoxicity against anti-MICA/MICB treated PL12 was 55-fold higher in untreated and 4.3-fold higher in IL-2 treated NK than untreated PL12. (FIGS. 4C and 4D) while the differences in cytotoxicity against untreated and anti-MICAS treated MP2 were not significant.

### Example 6: Differentiation of OSCSCs with Split-Anergized NK Cells Supernatants Results in their Susceptibility to NK Cell-Mediated ADCC Through Anti MICA/MICB Antibody

Treatment of OSCSCs and MP2 with split-anergized NK cells supernatant increases the surface expression of differentiation markers including MICA/MICB. To determine if differentiating oral and pancreatic tumors with split-anergized NK cells supernatants make them susceptible to NK cell mediated ADCC, OSCSCs, and MP2 were differentiated as described in Example 1, and cytotoxicity of untreated and IL-2 treated NK cells against untreated and MICA/MICB treated undifferentiated, split-anergized NK cells supernatant-differentiated, and undifferentiated/stem-like oral and pancreatic tumors were measured. The untreated and IL-2 treated NK cells mediated ADCC against OSCCSs and PL12 (FIGS. 5A and 5D). The undifferentiated OSCSCs and PL12 were highly susceptible to IL-2 treated NK cell mediated cytotoxicity, while the cytotoxicity against their supernatant-differentiated compartments showed 4-fold decrease in OSCSCs and 67-fold decrease in MP2. IL-2 treated NK cells showed higher level of cytotoxicity against anti-MICA/MICB treated differentiated OSCSCs and MP2 in comparison to untreated tumors, while the ADCC-mediated lysis did not happen against MP2 or OSCSCs (FIGS. 4B, 4C, 4E, and 4F) showing that differentiation of OSCSCs and MP2 with split-anergized NK cells supernatant, make them susceptible to NK cell mediated ADCC through anti-MICA/MICB antibody.

### Example 7: Antibodies Specific to MICA/MICB Increased IFN-γ Secretion by NK Cells when Cultured with Differentiated Oral Tumors Expressing MICA/MICB

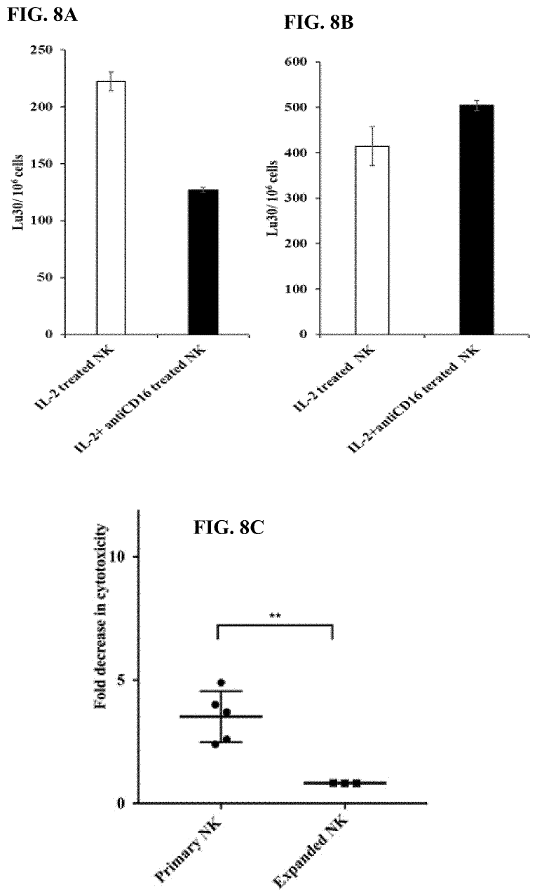

To determine whether anti-MICA/MICB antibody can increase the secretion of IFN-γ, NK cells were cultured with untreated or antiMICA/MCB treated OSCCs and OSCSCs. Untreated NK cells did not induce IFN-γ secretion without or with being co-cultured with tumor cells. As we shown previously combination of IL-2 and anti-CD16 mAb induced the highest level of IFN-γ by NK cells. When IL-2 treated NK cells were co-cultured with OSCCs and OSCSCs they secreted higher level of IFN-γ in comparison to the control groups (NK cells alone, with no tumor), and OSCSCs caused more secretion of IFN-γ than OSCCs. When IL-2 treated NK cells were cultured with antiMICA/MICB treated OSCCs they secreted more IFN-γ than NK cells cultured with untreated OSCCs while secreted IFN-γ in NK cells co-cultured with untreated OSCSCs and antiMICA/MICB treated OSCCs was not significantly different. The same trend was seen in three different separate experiment (FIGS. 8A-8C). The amount of IFN-γ secreted by NK cells treated with the combination of IL-2 and anti-CD16mAb was not significantly different in untreated and anti-MICA/MICB antibody treated OSCCs and OSCSCS.

### Example 8: Expanded NK Cells Target Both Undifferentiated and Differentiated Tumor Cells while Primary NK Cells Preferentially Target Undifferentiated/Stem-Like Population

Super-charged NK cells (“expanded NK cells”) have both high cytotoxicity and cytokine secretion abilities. When comparing the function and surface expression of primary and supercharged NK cells, they show different characteristics. Super-charged NK cells have high cytotoxicity against undifferentiated tumor cells, but their function against differentiated tumors was not studied. To compare the cytotoxicity of primary NK and expanded NK against differentiated and undifferentiated tumor cells, NK cells were expanded for 15 days and their cytotoxicity against OSCCs, OSCSCS, PL12 and MP2 was measured. While undifferentiated tumor cells were more susceptible to primary NK cell-mediated lysis, in comparison to differentiated compartments, expanded NK cells from the same donor were able to significantly target both differentiated and undifferentiated tumor cells. The cytotoxicity of expanded NK cells was 3 to 15-fold higher against OSCCs and 4.7-fold higher against PL12 in comparison to primary NK cells.

### Example 9: Combination of IL-2 and antiCD16 Treatment Induces Split Anergy in Primary NK Cells but not in Super-Charged NK Cells

A stage of NK cells maturation named “split anergy” that indicate reduced NK cell cytotoxicity in the presence of significant secretion of cytokines. Treatment of NK cells with IL-2 and anti-CD16 mAb, can induce split anergy in primary NK cells. To determine whether combination of IL-2 and anti-CD16 mAb treatment will decrease the cytotoxicity in expanded NK, freshly purified NK from healthy donors were expanded. After being in culture for 15 days, NK cells were purified from the same donors and primary and expanded NK cells were treated with IL-2 and the combination of IL-2 and anti-CD16 mAb for 18 hours and their cytotoxicity against OSCSCs was measured using standard 4-hours ⁵¹Cr release assay. The cytotoxicity of IL-2 and anti-CD16 treated primary NK cells against OSCSCs decrease 2.4 to 4.9-fold while the cytotoxicity in Expanded of IL-2 and anti-CD16 treated Expanded NK cells were almost the same as IL-2 treated expanded NK (FIG. 8).

### Example 10: Primary NK Cells Mediate Higher Levels of ADCC than Expanded NK Cells

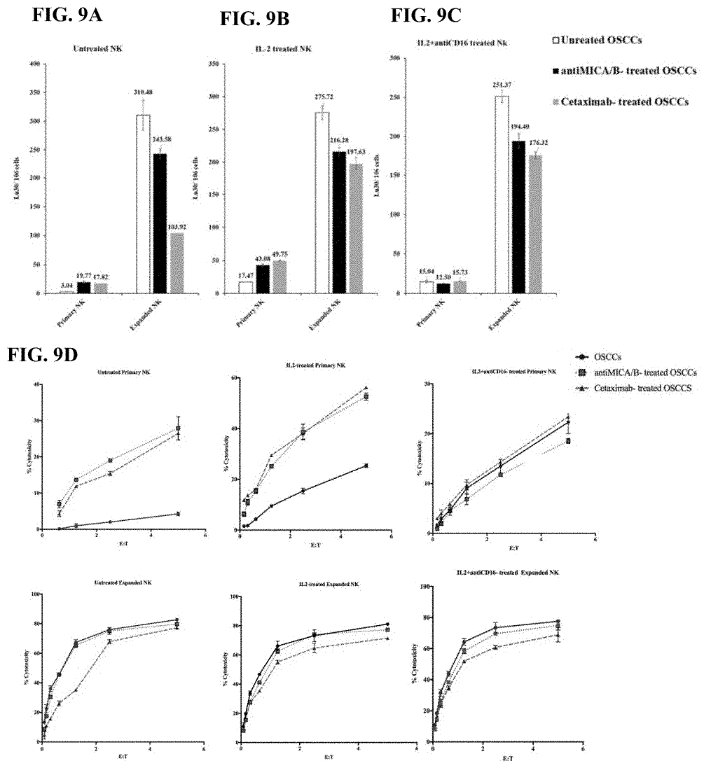

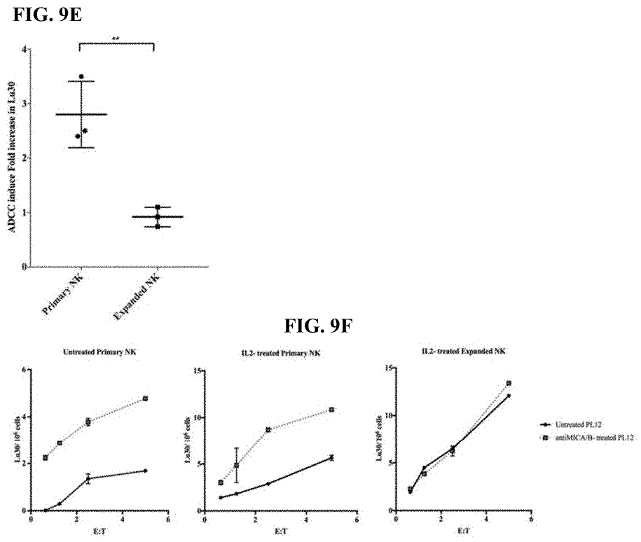

CD16 gets downmodulated on expanded NK cells. To study the ability of expanded NK cells in mediating ADCC, cytotoxicity of primary NK cells and expanded NK cells on day 15, from the same donor were measured against untreated, anti-MICA/MICB antibody, and Cetaximab treated OSCCs. While untreated and IL-2 treated primary NK cells mediated higher levels of cytotoxicity against anti-MICA/MICB antibody and Cetaximab treated OSCCs, than untreated tumors, expanded NK and IL2-reactivated NK expanded NK cells did not mediate ADCC against anti-MICA/MICB antibody or Cetaximab treated OSCCs. (FIGS. 9A, 9B, 9D and 9E). When the experiment repeated with PL12 tumors, the same results were seen (FIG. 9D). Both primary and expanded NK cells treated with the combination of IL-2 treated and anti-CD16 mAb, were not able to mediate ADCC against anti-MICA/MICB antibody treated OSCCs and PL12 (FIGS. 9C and 9F).

### Example 11: Treatment of NK Cells with Each AJ2 Probiotic Bacteria and D-Fucoidan Extracted from Mekabue Increased NK Cells IFN-γ Secretion Ability but does not Elevate NK Cell Mediated Cytotoxicity

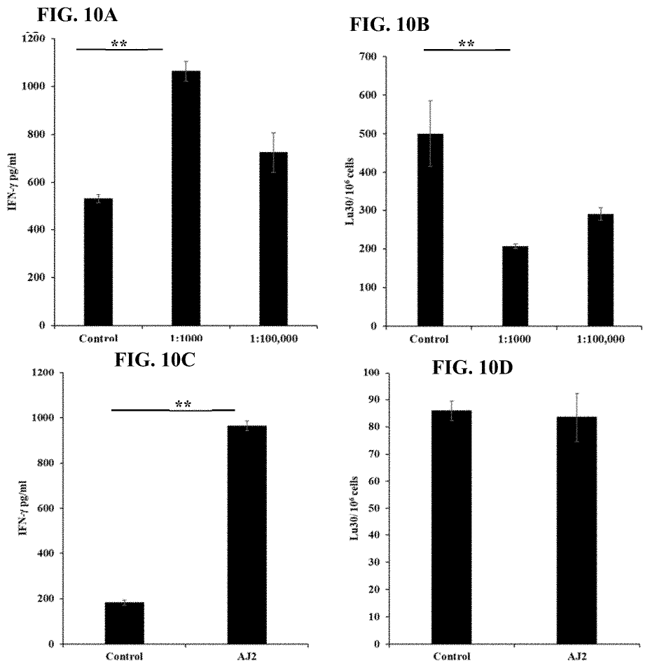

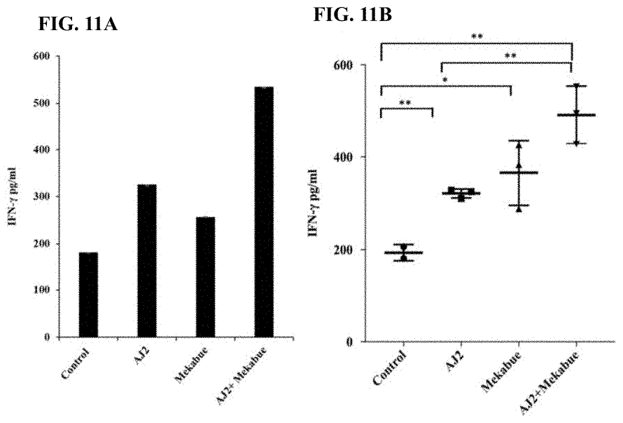

IFN-γ secreted by NK cells have a significant role in differentiation of tumor cells. To look at the strategy to increase the increase NK cell mediated production of IFN-γ, the effect of Fucoidan and AJ2 probiotic bacteria on function of NK cells was studied. To study the effect of Fucoidan on NK cells function, purified NK cells form healthy donors were treated with IL-2 and different concentrations of D-fucoidan extracted from Undaria pinnatifida known as Mekabue for 24 hours. Treatment of NK cells with Mekabue for 24 hours significantly increased their ability to secrete IFN-γ but a decreased their cytotoxicity, pushing the NK cells to a stage known as split-anergy (FIGS. 10A and 10B).

To study the effect of AJ2 probiotic bacteria on the function of NK cells, purified NK cells were treated with IL-2 for 18 hours and they were treated with the AJ2 probiotic bacteria for 24 hours. Activated NK cells with AJ2 induced higher level of IFN-γ, while their ability to mediated cytotoxicity was not changed. (FIGS. 10C and 10D).

### Example 12: AJ2 Probiotic Bacteria and Mekabue can Synergistically Induce IFN-γ Secretion in IL2-Treated NK Cells

To study the synergistic effect of AJ2 and Mekabue on IFN-γ induction by NK cells, purified NK cells form healthy donors were treated with IL-2 for 18 hours and then they were left untreated, treated with AJ2, or Mekabue, or both. The supernatant from the cultures was collected after 24 hours and IFN-γ as measured. NK cells treated with AJ2 or Mekabue induce significantly higher level of IFN-γ in comparison to control group (FIGS. 11A and 11B). Combination of AJ2 and Mekabue, significantly induced higher IFN-γ in comparison to the control and the NK cells treated with only AJ2 or Mekabue. (FIGS. 11A and 11B).

NK cells target poorly differentiated cells or stem cells with lower expression of key differentiation antigens. The link between the stage of maturation and differentiation of tumors and their sensitivity to NK cell-mediated lysis is discovered in this study. It is demonstrated herein that stem-like/poorly differentiated oral and pancreatic tumor cells were significantly more susceptible to NK cell-mediated cytotoxicity whereas, their differentiated counterparts were significantly more resistant. Furthermore, it is demonstrated herein that differentiated oral, pancreatic tumor cells, and cancer stem cells/poorly differentiated tumor cells differentiated in vitro with supernatants from split-anergized NK cells became resistant to NK cell-mediated cytotoxicity. Unlike the cancer stem cells (CSCs)/poorly differentiated tumor cells, both patient-derived differentiated tumor cells and split-anergized NK supernatant-differentiated tumor cells exhibited upregulated CD54, B7H1, and MHC class I surface expression and demonstrated decreased CD44 expression.

When it comes to NK cells immunity, MICA/MICB antigen plays an important role since it is a well-known ligand for NKG2D and it gets expressed upon stress, damage, viral infection or transformation of cells which act as a ‘kill me’ signal through the cytotoxic lymphocytes. It is demonstrated herein that MICA/MICB does not get upregulated on all tumor cells, but it is correlated with the differentiation stages of the cells. It is presented herein for the first time that differentiated oral, pancreatic tumor cells, and cancer stem cells/poorly differentiated tumor cells differentiated in vitro with supernatants from split-anergized NK cells express a higher level of MICA/MICB in comparison to their stem-like/undifferentiated counterpart. Primary NK cells preferentially target stem-like/undifferentiated cells. Accordingly, the surprising observation that well-differentiated cells express a higher level of MICA/MICB, which is a ligand for an activator receptor on NK cells, indicates the novel roles of MICA/MICB ligands and its receptors. Since there was a difference in the pattern of MICA/MICB expression in stem-like/undifferentiated and well-differentiated oral and pancreatic tumor, it was studied whether NK cells mediate ADCC differentially against these tumors based on the antibody specific to MICA/MICB. It is demonstrated herein that antibodies specific to MICA/MICB increased NK cell-mediated ADCC against well-differentiated and cancer stem cells/poorly differentiated tumor cells differentiated in vitro with supernatants from split-anergized NK cells while stem-like/undifferentiated were not targeted by untreated and IL-2 treated primary NK cells through ADCC which correlated with the expression level of MICA/MICB. When NK cells were treated with antiCD16 mAb, NK cells were not able to mediate ADCC since the CD16 receptor was masked by the antibody. It is further presented herein that antibody specific to MICA/MICB increased IFN-γ secretion by NK cells when cultured with differentiated Oral tumors expressing MICA/MICB but not stem-like/undifferentiated Oral tumor cells. When NK cells were cultured with target cells and the antibody specific to the antigen they present, the cytokines such as TNFα, IL-6, IFN-γ or chemokines such as IL-8 and MCP-1 were found to be significantly enhanced.

It was also delineated herein the underlying differences between the functions of primary and Expanded NK cells in direct cytotoxicity and ADCC. NK cells were expanded by IL-2, antiCD16 mAb, and AJ2 probiotic bacteria activation and using Osteoclast as feeder cells. This strategy leads to NK cells called “Super Charged NK cell”, having high cytotoxicity and high level of IFN-γ secretion. When the cytotoxicity of NK cells against stem-like/undifferentiated oral and pancreatic tumors with their well-differentiated counterpart were compared, it was discovered that Expanded NK cells target both undifferentiated and differentiated tumor cells while primary NK cells preferentially target undifferentiated/Stem-like population. The cytotoxicity of expanded NK cells was 3 to 15-fold higher against OSCCs and 4.7-fold higher against PL12 in comparison to primary NK cells showing that the extend well-differentiated tumor cells can become sensitive to Expanded NK cell-mediated cytotoxicity can be different based on the biology of these cells and maybe their resistance to primary NK cells mediated cytotoxicity. Expanded NK cells express a higher level of NKG2D than primary NK cells. As presented above, MICA/MICB, one of the NKG2D ligands, expresses highly on well-differentiated cells, which also express a high level of MHC-I. Therefore, since Expanded NK cells express a higher level of NKG2D, the NKG2D-MICA/MICB mediated lysis become the dominant mechanism of cytotoxicity in expanded NK cells leading to targeting both undifferentiated and differentiated tumor cells.

The stage of maturation in NK cells called split anergy can be initiated by targeting different receptors on NK cells including the CD16 receptor. As described above, when NK cells were treated with the combination of IL-2 and antiCD16 mAb, their cytotoxicity decrease but their ability to secrete IFN-γ significantly increase in comparison to IL-2 treated NK cells. Furthermore, it is presented herein that spilt anergy occurs in the primary NK cells, but not in the Expanded NK cells. Treating Expanded NK cells with the combination of IL-2 and antiCD16 did not result in a decrease in their cytotoxicity. CD16 gets downregulated on the surface of expanded NK cells and this explains why antiCD16 treatment in Expanded NK cells cannot push them towards the split anergy stage. Since the level of CD16 is different on primary and Expanded NK cells, ADCC in primary and Expanded NK cells was compared. When the level of ADCC was measured in untreated and IL-2 treated NK cells against well-differentiated Oral tumors treated with antiMICA/MICB antibody and also Cetaximab (antibody against EGFR receptor), despite primary NK cells, Expanded NK cells were not able to mediate ADCC and even some levels of inhibition was seen in antibody treated tumor cells in comparison to the untreated cells. The lower level of ADCC of Expanded NK cells is due to the downmodulation of CD16 receptor on their surface. Although Expanded NK cells express some levels of CD16, a higher level of CD16 is required for NK cells to be able to mediate ADCC as it is known that during HIV-1 infection, NK cells are known to express low levels of CD16 and exhibit reduced ADCC.

Induction of split anergy in NK cell effector function drives differentiation of healthy, as well as transformed stem-like cells. Differentiation of tumor cells is one of the important role of NK cells since the differentiated tumor micro environment become less invasive and more susceptible to chemotherapeutics. Cytokines that are secreted by NK cells, primarily IFN-γ and TNF-α, are responsible for the differentiation of cancer stem cells (CSCs) and result in the increase in differentiation antigens such as MHC class I, CD54, B7H1, and MICA and decrease in CD44. Accordingly, exposing oral and pancreatic cancer stem cells to IFN-γ secreted by NK cells activated with IL-2 and CD16, resulted in upregulation of differentiation the same differentiation markers a respectively the differentiated tumor cells, lose their cytotoxicity against NK cells. These results and the work on AJ2 probiotic bacteria illustrate the profound capability probiotic bacteria has on NK cells to induce a significant increase in cytokine secretion, known as split anergy. AJ2 is a combination of 8 strains of probiotic bacteria for their ability to induce significant secretion of IFN-γ when added to IL-2 or IL-2+anti-CD16mAb treated NK cells. The ratio of bacteria added to create sAJ2 was adjusted to yield a ratio of IFN-γ to IL-10 for when cells are activated with IL-2 or IL-2+anti-CD16mAb without bacteria. This ratio was established to obtain a similar ratio when NK cells are activated with IL-2+anti-CD16mAb without bacteria since this NK treatment provided increased differentiation of stem cells. IL-10, an anti-inflammatory cytokine, was taken into consideration to balance the significant amount of IFN-γ secreted by cells during the process of differentiation. This combination of bacterial strains was selected due to its optimal induction of pro- and anti-inflammatory cytokine and growth factors by the NK cells.

Fucoidan is a sulfated polysaccharide, can be extracted from different species of brown algae and brown seaweed. This compound has been known to have immunomodulatory effects on immune cells. Although studies have shown that Fucoidan can decrease tumor size, have antitumor activities, and lead to higher survival in tumor-induced mouse, the exact role of Fucoidan on NK cells function has not been well studied. It is presented herein that Mekabue extracted Fucoidan increased NK cells IFN-γ secretion ability but decreased NK cell-mediated cytotoxicity which is a profile of split anergized NK cells. Seeking strategies to push NK cells to secrete the highest level of IFN-γ, it was investigated herein the synergistic effect of Mekabue and AJ2 probiotic bacteria on NK cell-mediated IFN-γ secretion. NK cells treated with AJ2 or Mekabue induce significantly higher level of IFN-γ in comparison to untreated, sAJ2, or Mekabue treated IL-2 treated NK cells. NK cells express different families of Toll-like receptors (TLRs). The gram-positive bacteria in the probiotic bacteria can trigger NK cells TLRs via their cell wall components. A study showed that cytokine induction by both B. breve and the lactobacilli is strongly dependent on TLR9 since blocking of TLR9 resulted in decreased production of IL-10 and IFN-γ in PBMCs. Fucoidan from seaweeds is independent ligands for TLR-2 and TLR-4. Accordingly, NK cells produce a higher level of IFN-γ in presence of both AJ2 probiotic bacteria and Mekabue because these compound target different family of TLRs on NK cells.

In conclusion, differentiation stages of pancreatic cancer cells correlated directly with the resistance to NK cell-mediated cytotoxicity and expression of key surface antigens. Differentiation by NK cells is very important in effective targeting of cancer stem cells/undifferentiated tumor cells. As IFN-γ plays a critical rule in differentiation, treatment strategy to push NK cells to produce higher levels of IFN-γ is a critical step in eliminating tumors. Combination of probiotic AJ2 bacteria with fucoidan extracted from Mekabue seaweed can cause higher secretion of IFN-γ by NK cells. Oral and pancreatic tumor cells have a specific pattern of MICA/MICB antigen expression as differentiated tumor cells express higher levels of MICA/MICB than stem-like/undifferentiated tumor cells. Since well differentiated cells express higher levels of MICA/MICB, NK cells mediate higher levels of ADCC through antibody specific to MICA/MICB against these cells than their undifferentiated compartments. Furthermore, Primary and Expanded NK cells have very different characteristic and biological functions. Accordingly, all diverse functions of different subsets NK cells provide a novel way of developing NK cell-immunotherapeutic approaches.

### Example 13: Materials and Methods for Examples 14-23

### Cell Lines, Reagents, and Antibodies

RPMI 1640 supplemented with 10% Fetal Bovine Serum (FBS) (Gemini Bio-Products, CA) was used for the cultures of human NK cells, and oral squamous carcinoma stem-like cells (OSCSCs). RPMI 1640 supplemented with 10% Fetal Bovine Serum (FBS) (Gemini Bio-Products, CA) was used for the cultures the cells isolated from hu-BLT mice tissues. MiaPaCa-2 (MP2), PL12, BXPC3, HPAF, and Capan were cultured with DMEM supplemented with 10% FBS. DMEM supplemented with 10% FBS was used to culture pancreatic tumor cells isolated from hu-BLT mice pancreas. Recombinant IL-2 (rhlL-2) was obtained from NIH-BRB. Flow cytometry and other antibodies used in the study were obtained from Biolegend (San Diego, Calif.). Monoclonal antibodies to TNF-α and IFN-γ were prepared and 1:100 dilutions were found to be the optimal concentration to use for blocking experiments. NAC at 20 mM was prepared using sterilized distilled water at pH 7-7.2 and, was diluted using DMEM media to have final concentration of 20 nM.

Human pancreatic cancer cell lines Panc-1, MIA PaCa-2 (MP2), BXPC3, HPAF, Capan were generously provided by Dr. Guido Eibl (UCLA David Geffen School of Medicine) and PL12 was provided by Dr. Nicholas Cacalano (UCLA Jonsson Comprehensive Cancer Center). Panc-1, MP2 and BXPC3 were cultured with DMEM in supplement with 10% FBS and 2% Penicillin-Streptomycin (Gemini Bio-Products, CA). HPAF, Capan and PL12 were cultured in RMPI 1640 medium supplemented with 10% FBS and 2% Penicillin-Streptomycin. Recombinant human IL-2 was obtained from NIH-BRB. Recombinant human TNF-α rand IFN-γ were obtained from Biolegend (San Diego, Calif.). Antibodies to CD16 were purchased from Biolegend (San Diego, Calif.). Anti-MHC class I was prepared and 1:100 dilution was found to be the optimal concentration to use. Fluorochrome-conjugated human and mouse antibodies for flow cytometry were obtained from Biolegend (San Diego, Calif.). Monoclonal antibodies to TNF-α were prepared from ascites of mice injected with TNF-α hybridomas, after which the antibodies were purified and specificity determined by both ELISA and functional assays against rh TNF-α. Monoclonal IFN-γ antibodies were prepared in rabbits, purified and specificity determined with ELISA and functional assays against rIFN-γ. 1:100 dilution of anti-TNF-α and anti-IFN-γ antibodies was found to be the optimal concentration to block rhTNF-α and rhIFN-γ function. The human NK, CD3+ T cells and monocytes purification kits were obtained from Stem Cell Technologies (Vancouver, Canada). Propidium iodide and N-Acetyl Cysteine (NAC) were purchased from Sigma Aldrich (St. Louis, Mo.). Cisplatin and paclitaxel were purchased from Ronald Reagan UCLA Medical Center Pharmacy (Los Angeles, Calif.).

### Purification of Human NK Cells and Monocytes

Written informed consents approved by UCLA Institutional Review Board (IRB) were obtained and all procedures were approved by the UCLA-IRB. NK cells and monocytes were negatively selected and isolated from PBMCs using the EasySep® Human NK cell enrichment kit and monocyte isolation kit, respectively purchased from Stem Cell Technologies (Vancouver, BC, Canada). Isolated NK cells and monocytes were stained with anti-CD16 and anti-CD14 antibody, respectively, to measure the cell purity using flow cytometric analysis.

### Analysis of Human Pancreatic Cancer Cells Growth in Immune-Deficient (NSG) and Humanized-BLT Mice

Animal research was performed under the written approval of the UCLA Animal Research Committee (ARC). Humanized-BLT (hu-BLT; human bone marrow/liver/thymus) mice were prepared as previously described.

In vivo growth of pancreatic tumors were done by orthotopic cell implantation into 8-10 week-old NSG mice or hu-BLT mice pancreas. To establish orthotopic tumors, mice were anesthetized using isoflurane followed by 2 cm of the incision on the lower right abdomen. Once the spleen was exposed, spleen was pulled out as pancreas in lying under the spleen. Spleen was holded using sterilized forceps and the pancreas was exposed (laparotomy). Tumor cells were then transferred by direct injection with 10 μl HC Matrigel (Corning, N.Y., USA) using insulin syringe with 28 G needle in the pancreas. Mice were monitored for tumor growth by palpating the abdominal site. 7 to 10 days after the surgery mice received 1.5×10⁶ super-charged NK cells via tail vein injection. Mice were fed AJ2 (5 billion/dose) orally, similar to how humans ingest probiotics. The first dose of AJ2 was given one or two weeks before tumor implantation and was continued throughout the experiment every 48 hours. Mice were euthanized when signs of morbidity were evident. Pancreas, pancreatic tumors, bone marrow, spleen, and peripheral blood were harvested from mice at the end of the experiment or when tumor size reached 2 cm diameter.

### Analysis of Human Oral Cancer Cells Growth in Immunodeficient and Humanized Mice

To establish orthotopic tumors, mice were anesthetized using isoflurane and oral tumor cells were then injected in oral floor by direct injection with 10 μl HC Matrigel (Corning, N.Y., USA). 7 to 10 days after the oral tumor injections, mice received 1.5×10⁶ super-charged NK cells via tail vein injection. Mice were fed AJ2 (5 billion/dose) orally, similar to how humans ingest probiotics. The first dose of AJ2 was given one or two weeks before tumor implantation and was continued throughout the experiment every 48 hours. Mice were euthanized when signs of morbidity were evident. Peripheral blood was harvested from mice at the end of the experiment or when tumor size reached 2 cm diameter.

Cell Dissociation and Cell Culture of Tissues from Hu-BLT and NSG Mice

The pancreas and/or pancreatic tumor harvested from NSG and hu-BLT mice were immediately cut into 1 mm³ pieces and placed into a digestion buffer containing 1 mg/ml collagenase IV, 10 U/ml DNAse I, and 1% bovine serum albumin (BSA) in DMEM media, and incubated for 20 minutes at 37° C. oven on a 150 rpm shaker. After digestion, the sample was filtered through a 40 mm cell strainer and centrifuged at 1500 rpm for 10 minutes at 4° C. The pellet was re-suspended in DMEM media and cells were counted. To obtain single-cell suspensions from BM, femurs were cut from both ends and were flushed from one end to other using RPMI media, BM cells was filtered through a 40 mm cell. To obtain single-cell suspensions from spleen, spleen was smashed until no big piece was left and sample was filtered through a 40 mm cell and centrifuged at 1500 rpm for 5 minutes at 4° C. The pellet was re-suspended in ACK buffer to remove the red blood cells for 2-5 mins followed re-suspension in RMPI media and centrifuged at 1500 rpm for 5 minutes at 4° C. Peripheral blood mononuclear cells (PBMCs) were isolated using ficoll-hypaque centrifugation of heparinized blood specimens. The buffy coat containing PBMCs were harvested, washed and re-suspended in RPMI 1640 medium.

Purification of NK Cells, CD3+ T Cells, and Monocytes from Hu-BLT Mice

NK cells from hu-BLT mice splenocytes were isolated using the human CD56+ selection kit (Stem Cells Technologies, Canada). Monocytes from hu-BLT mice BM cells were positively selected from BM using human CD14 isolation kit (eBioscience, San Diego, Calif.). Isolated NK cells and monocytes were stained with anti-CD16 and anti-CD14 antibody, respectively, to measure the cell purity using flow cytometric analysis.

### Generation of Osteoclasts and Expansion of Human and Hu-BLT NK Cells

Purified monocytes both form human peripheral blood and hu-BLT mice BM cells were cultured in alpha-MEM medium containing M-CSF (25 ng/mL) and RANKL (25 ng/mL) for 21 days, or otherwise specified. The medium was refreshed every 3 days with fresh alpha-MEM containing M-CSF and RANKL. Human purified and hu-BLT NK cells were activated with rh-IL-2 (1000 U/ml) and anti-CD16mAb (3 ug/ml) for 18-20 hours before they were co-cultured with osteoclasts and sonicated AJ2 for NK cells expansion. The medium was refreshed every 3 days with RMPI containing rh-IL-2 (1500 U/ml).

### Enzyme-Linked Immunosorbent Assays (ELISAs) and Multiplex Cytokine Assay

Human ELISA kits for IFN-γ were purchased from Biolegend (San Diego, Calif.). The assay was conducted as described in the manufacturer's protocol. The plates were read in a microplate reader, at 450 nm to obtain absorbance values (Biolegend, ELISA manual). To analyze and obtain the cytokine and chemokine concentration, a standard curve was generated by either two or three-fold dilution of recombinant cytokines provided by the manufacturer.

The levels of cytokines and chemokines were examined by multiplex assay, which was conducted as described in the manufacturer's protocol for each specified kit. Analysis was performed using a Luminex multiplex instrument (MAGPIX, Millipore, Billerica, Mass.) and data was analyzed using the proprietary software (xPONENT 4.2, Millipore, Billerica, Mass.).

### Surface Staining and Cell Death Assays

Staining was performed by labeling the cells with antibodies as described previously. Briefly, the cells were washed twice with ice-cold PBS/1% BSA. Predetermined optimal concentrations of specific human flow cytometric antibodies were added to 1×10⁴ cells in 50 μl of cold-PBS/1% BSA and cells were incubated on ice for 30 min. Thereafter cells were washed in cold PBS/1% BSA and brought to 500 μl with PBS/1% BSA. Flow cytometry analysis was performed using Beckman Coulter Epics XL cytometer (Brea, Calif.) and results were analyzed in FlowJo vX software (Ashland, Oreg.).

### ⁵¹Cr Release Cytotoxicity Assay

The ⁵¹Cr release assay was performed as described previously. OSCSCs were used as target cells to assess NK cell-mediated cytotoxicity because these cells are the most susceptible cells to NK cell-mediated cytotoxicity. Briefly, different numbers of effector cells were incubated with ⁵¹Cr-labeled target cells. After a 4-hour incubation period the supernatants were harvested from each sample and counted for released radioactivity using the gamma counter. The percentage specific cytotoxicity was calculated using the following formula:

$$\% Cytotoxicity = \frac{Experimental cpm - spontaneous cpm}{Total cpm - spontaneous cpm}$$

Lytic unit 30/106 is calculated by using the inverse of the number of effector cells needed to lyse 30% of tumor target cells×100.

### In-Vitro MP2 Cancer Stem Cell Differentiation

Differentiation of MP2 tumors was conducted as described previously. NK cells were treated with a combination of anti-CD16mAb (3 μg/mL) and IL-2 (1,000 U/mL) for 18 hours before supernatants were removed and used for differentiation experiments. The amounts of IFN-γ produced by activated NK cells were assess with IFN-γ ELISA (Biolegend, CA, USA). MP2 cells were differentiated with gradual daily addition of increasing amounts of NK cell supernatants (of corresponding treatments). On average, to induce differentiation, a total of 3,500 pg of IFN-γ containing supernatants were added for 4 days to induce differentiation and resistance of MP2 tumor cells to NK cell-mediated cytotoxicity. Afterwards, target cells were washed with 1×PBS, detached and used for experiments.

### Statistical Analysis

An unpaired, two-tailed student t-test was performed for the statistical analysis. One-way ANOVA using Prism-7 software was used to compare different groups. (n) denotes the number of mice used for each condition in the experiment. The following symbols represent the levels of statistical significance within each analysis, ***(p-value<0.001), **(p-value 0.001-0.01), *(p-value 0.01-0.05).

### Example 14: The Stage of Differentiation in Pancreatic Tumors Correlates with Susceptibility to NK Cell-Mediated Cytotoxicity; Loss of NK Cell Cytotoxicity after NK Cell Receptor Triggering

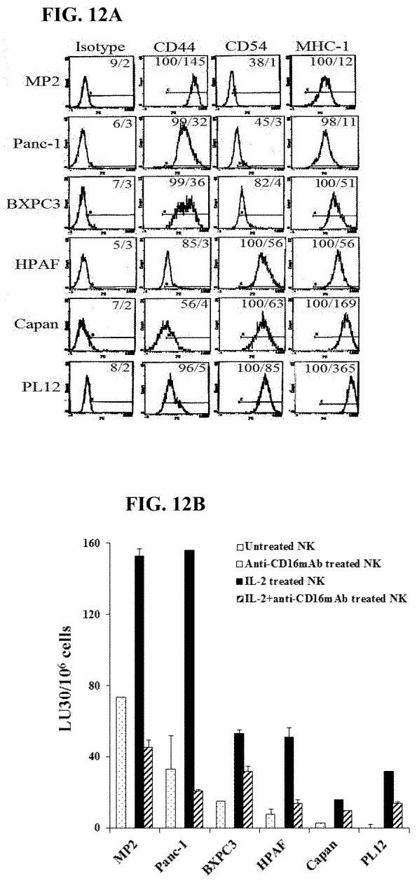

Six pancreatic tumor cells were used to determine surface expression, susceptibility to NK cell mediated cytotoxicity and secretion of cytokines when cultured with NK cells. Poorly differentiated MP-2 and Panc-1 expressed higher amounts of CD44 and moderate or low levels of MHC class I and CD54. Moderately differentiated BXPC3 and HPAF expressed moderate to high levels of CD44 and CD54 and higher levels of MHC-class I. Well differentiated Capan and PL12 had much lower levels of CD44 and higher levels of CD54 and MHC class I (FIG. 12A). A direct correlation between the stage of differentiation and susceptibility to NK cell mediated cytotoxicity was observed in pancreatic tumor cells. Undifferentiated MP2 and Panc-1 exhibited the highest whereas PL-12 and Capan well differentiated tumors demonstrated the lowest sensitivity to NK mediated lysis (FIG. 12B). Moderately differentiated BXPC3 and HPAF demonstrated intermediate sensitivity to NK cell lysis (FIG. 12B). Thus, there was a direct correlation between increased susceptibility to NK-mediated cytotoxicity and poor differentiation of pancreatic tumors.

Among six different pancreatic tumor types (FIGS. 12A and 12B), each identified and characterized previously to be at different stages of differentiation morphologically and pathologically; MiaPaCa-2 (MP2) at stem-like/poorly differentiated stage and PL12 at well differentiated stage representing two extreme stages of differentiation were chosen for use in our in vivo experiments described below (FIGS. 12A and 12B). The results indicated that MP2 tumors expressed higher CD44 and lower MHC-class I and CD54 (FIG. 12A), and were highly susceptible to NK cell-mediated cytotoxicity (FIG. 12B), whereas PL12 tumors had lower CD44 but higher CD54 and MHC-class I surface expressions (FIG. 12A), and were resistant to NK cell-mediated cytotoxicity (FIG. 12B). Similar trends were also seen with other pancreatic tumor types (FIGS. 12A and 12B). It is also shown herein the abovementioned criteria for tumor differentiation in oral and glioblastoma, and more recently in Melanoma and Lung tumors.

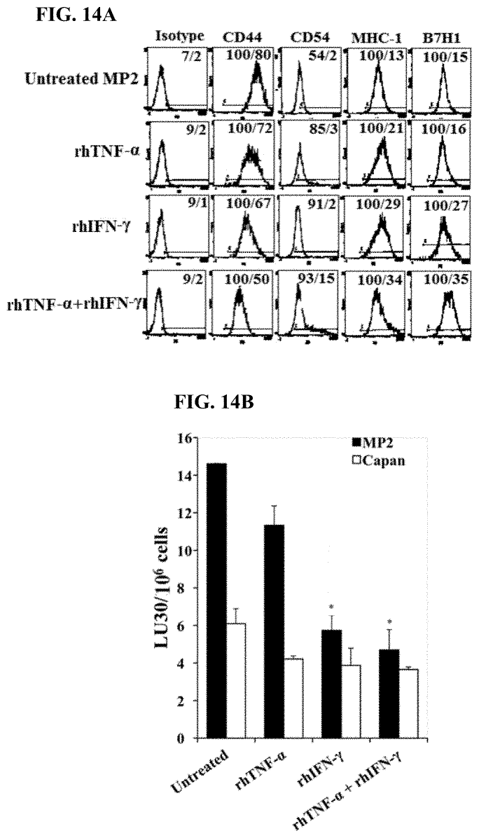

NK-differentiated MP2 tumors exhibited similar surface phenotype to well-differentiated PL12 and Capan tumors, and they too were resistant to NK cell mediated cytotoxicity (FIGS. 12A and 12B). NK cells mediated differentiation of MP2 tumors through the functions of secreted and membrane-bound IFN-γ and TNF-α. Moreover, the significance of IFN-γ and TNF-α receptors, as well as rhIFN-γ and/or rhTNF-α in mediating MP2 tumor differentiation is shown (FIGS. 14A and 14B).

### Example 15: Curtailed Pancreatic Tumor Growth and Lack of Metastasis and Long-Term Survival of NSG Mice after Implantation of NK-Supernatant Differentiated MP2 Tumors and Patient-Derived Differentiated PL12 Tumors

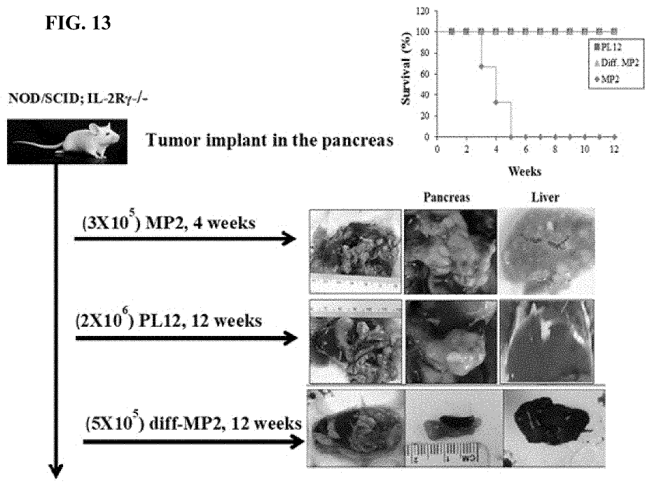

MP2 tumors (3×10⁵ tumors) implanted in the pancreas of NSG mice grew within 3-4 weeks and metastasized to liver and caused death of the mice (FIG. 13) (n=3), whereas mice injected with greater numbers of PL12 tumors (2×10⁶) generated no or very small tumors within 12 weeks and tumors did not metastasize nor caused death of the mice (FIG. 13) (n=3). Injection of NK-differentiated MP2 tumors (5×10⁵) to pancreas did not exhibit visible tumor growth nor tumors metastasized to the liver and all mice survived at 12 weeks when the experiments were terminated (FIG. 13) (n=3).

### Example 16: Combination of rhTNF-α and rhIFN-γ Induce Differentiation and Resistance of MP2 Cells to NK Cell Mediated Cytotoxicity

Stem-like/undifferentiated MP2 and well differentiated Capan pancreatic tumor cells were treated with rhTNF-α and rhIFN-γ and their susceptibility to NK cell mediated lysis was assessed in a standard 4-hour 51Cr release assay. As shown in FIG. 14A, the combination of rhTNF-α and rhIFN-γ were able to upregulate CD54, MHC-1 and B7H1 and down modulate CD44 in MP-2 tumors. Both rhTNF-α and rhIFN-γ were able to increase surface expression of CD54 and MHC-class I, however, only rhIFN-γ was able to upregulate B7H1 (FIG. 14A). The addition of rhTNF-α to MP2 was able to induce moderate resistance against NK cell mediated cytotoxicity whereas rhIFN-γ induced significant resistance (FIG. 14B). There was less lysis of Capan tumors by the NK cells and treatment with rhIFN-γ and rhTNF-α induced moderate resistance in these cells (FIG. 14B).

### Example 17: Differences in the Frequencies of NK Cells in Humans and Hu-BLT Mice

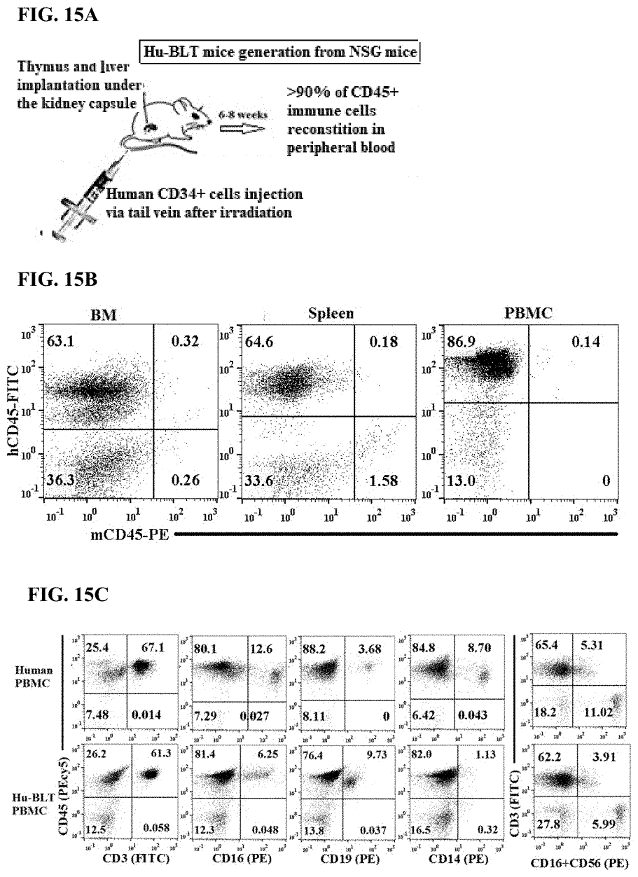

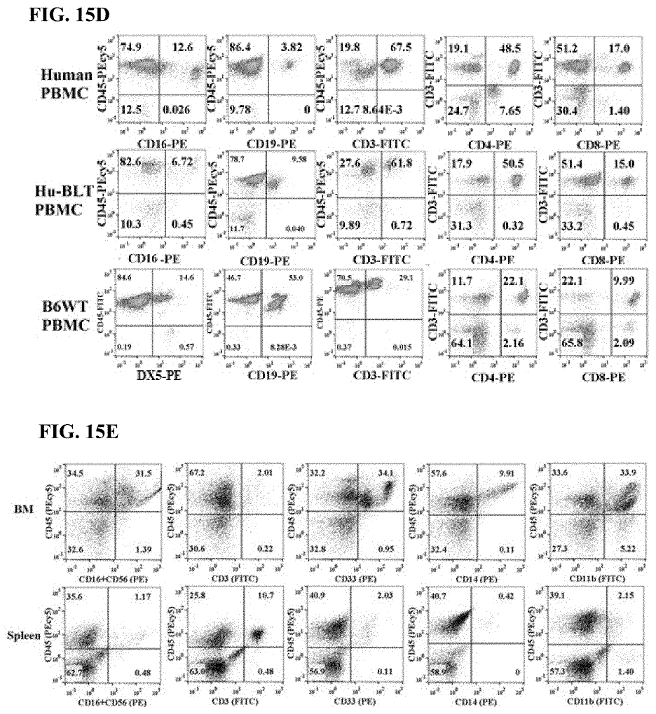

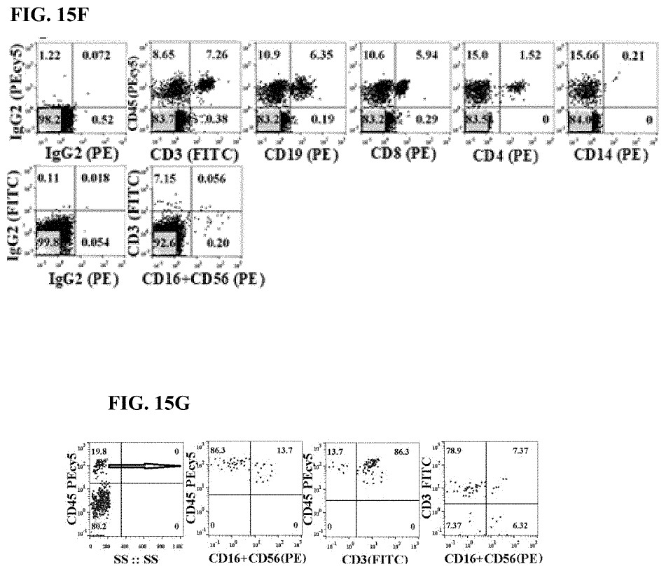

Hu-BLT mice that were reconstituted with the human immune system, exhibited greater than 90% reconstitution with huCD45+ immune cells in different tissue compartments (FIGS. 15A and 15B). Similar to humans in which a range of frequencies can be seen in peripheral blood NK cells between donors, there are also variable percentages of NK cells in peripheral blood of hu-BLT mice reconstituted with different donor immune cells. Based on the number of hu-BLT mice tested so far, on average there may be a lower percentage of NK cells in peripheral blood of hu-BLT mice as compared to human donor peripheral blood, however, because of the limited availability of thymus and liver tissues, in addition to cost and labor involved in generating these mice, they are not as readily available as the human donor blood to establish a population base range for the percentages of peripheral blood NK cells (FIGS. 15C and 15D; Table 1). However, based on the analysis of different immune cell subsets in different tissue compartments in hu-BLT mice, it was discovered herein great reconstitution and infiltration of human immune subsets in peripheral blood (FIGS. 15C and 15D; Table 1), BM (FIG. 15E), spleen (FIG. 15E), pancreas (FIG. 15F) and even gingiva (FIG. 15G) which is a very difficult compartment to assess for immune infiltration. Indeed, when gingival cells were cultured for 7 days, great infiltration of huCD45+ immune cells and different immune subsets could be ascertained clearly (FIG. 15G). Identical percentages of T cell subsets between human and hu-BLT mice peripheral blood were found (FIG. 15D; Table 1). When comparing immune cell subsets between hu-BLT, human and murine peripheral blood, similar but not identical frequencies of subsets of lymphocytes can be found between hu-BLT and human donors, and both were very different from those obtained from murine peripheral blood (FIG. 15D; Table 1).

TABLE 1

| | CD45⁺CD16⁺CD56⁺ | CD45⁺CD19⁺ | CD45⁺CD3⁺ | CD45⁺CD3⁺CD4⁺ | CD45⁺CD3⁺CD8⁺ |
|---|---|---|---|---|---|
| Hu-PBMC | 14.4% | 4.2% | 77% | 72% | 24.9% |
| Hu-BLT PBMC | 7.5% | 10.8% | 69% | 60% | 22.6% |
| B6WT PBMC | 14.7% | 53% | 27% | 65% | 17.4% |

### Example 18: NK-Differentiated MP2 Tumors Did not Grow Visible Tumors in the Pancreas of Hu-BLT Mice

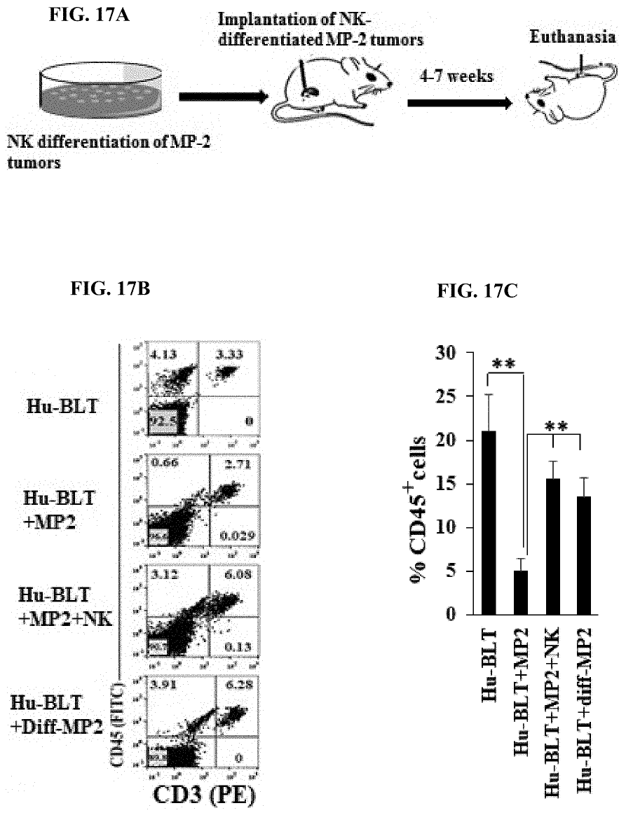

Hu-BLT mice (FIGS. 15A-15G show the details of the immune subsets in different tissue compartments of hu-BLT and their comparison with the human and mouse immune subsets in peripheral blood) were implanted with undifferentiated MP2 tumors (FIG. 16A) and those differentiated with NK-supernatants as described before and here (FIG. 17A) in the pancreas, and their growth dynamics and overall effect on mice were studied. MP2 tumors grew rapidly and formed palpable tumors in the pancreas, and mice exhibited all the signs of morbidity within 6-7 weeks, and upon sacrifice at week 7, they exhibited tumors which spanned the entire abdomen and enveloped the spleen, stomach and a portion of intestines (FIG. 16B-top panel). When NK-differentiated MP2 tumors were implanted in mice, no tumors were palpable or visible up until the day of sacrifice, and mice did not exhibit any signs of morbidity, and upon sacrifice at week 7, no tumors could be seen visually (FIG. 16B-bottom panel). In in vitro cell cultures, NK-differentiated MP2 tumors similar to patient derived PL12 differentiated tumors grew slower when compared to undifferentiated MP2 tumors.

### Example 19: Frequencies of Immune Subsets in the Pancreas

The majority of infiltrating human immune cells in the pancreas was CD3+ T (54%) and B cells (43.3%), with CD8+ T cells constituting the larger proportions of the T cells (approximately 80%) than CD4+ T cells (approximately 20%) (Table 2; FIG. 15F). NK and CD14+ cells constituted minor subpopulations of immune cells in the pancreas of healthy hu-BLT mice (Table 2; FIG. 15F).

TABLE 2

| Immune cells in the Hu-BLT pancres | % in CD45+ immune cells |
|---|---|
| CD3+ cells (T cells) | 53.7% |
| CD8+ cells | 42.24% |
| CD4+ cells | 10.76% |
| CD19+ cells (B cells) | 43.32% |
| CD14+ cells (Monocytes) | 1.5% |
| CD16+ cells (NK cells) | 1.44% |

### Example 20: Single Injection of NK Cells Inhibited Tumor Growth in Mice Implanted with MP2 Tumors

Mice implanted with MP2 tumors and injected with 1 to 1.5×10⁶ super-charged NK cells with potent cytotoxic and cytokine secretion capabilities (FIG. 16A) exhibited no or substantially smaller tumors, without the involvement of other organs or signs of morbidity (FIG. 16B-middle panel). Due to the death of tumor bearing mice by fast growing tumors in 7 weeks after tumor implantation, the time of sacrifice was shortened to 4-5 weeks after tumor implantation to be able to study the pancreas and the dynamics of immune infiltration in the pancreas. Greater than 2 fold huCD45+ immune cells were seen in the pancreas of either NK-injected tumor-bearing mice or NK-differentiated tumor-implanted mice or in the healthy control mice when compared to those from tumor-bearing mice (FIGS. 17B and 17C). In addition, most of the pancreatic huCD45+ immune cells in MP2 implanted mice were CD3+ T cells (FIGS. 16C and 17B), whereas tumor-bearing mice which received NK cells or mice with implanted NK-differentiated tumors or control healthy pancreas exhibited relatively lower percentages of CD3+ T cells (FIGS. 16C and 17B) and higher percentages of NK cells in the pancreas (FIG. 16D). Likewise, greater than 2 fold increases in CD16+CD56+NK cells within the pancreas was seen in either NK-injected tumor-bearing mice or in the pancreas of healthy control mice with no tumor implantation, when compared to those from tumor-bearing mice (FIG. 16D).

Unlike tumor-bearing mice, when mice were fed AJ2 1-2 weeks before tumor implantation, and injected with allogeneic or autologous super-charged NK cells (FIG. 19A and please see below), tumors were not palpable (FIG. 18A), and their tumor weights remained substantially low (FIG. 19B). No statistically significant differences in tumor weight could be observed between NK or NK injected/AJ2 fed mice, even though a slight decrease in the average tumor weight could be seen between the two (FIG. 19B). This is likely due to the significant decrease already seen with NK injection alone in tumor bearing mice. Indeed, sera from the peripheral blood of NK-injected or NK injected/AJ2 fed tumor-bearing mice exhibited 2.73 and 4.8-fold more IFN-γ respectively when compared to tumor-alone bearing mice (FIG. 19C). Similarly, mice implanted with NK-differentiated MP2 tumors (FIG. 17A) did not have palpable tumors, and blocking MP2 differentiation with anti-IFN-γ and anti-TNF-α antibodies (FIG. 18B) resulted in the inhibition of tumor differentiation and generation of palpable tumors with higher tumor weights (FIG. 19D).

When pancreas were removed, dissociated and equal numbers of cells were cultured from tumor-bearing mice which did not receive NK injection, attached colonies of tumors could be seen in 24-48 hours and they grew rapidly thereafter, whereas those injected with allogeneic NK cells (FIGS. 18C-18I) or autologous NK cells (FIG. 18G) did not exhibit colonies initially, but a few were visible after day 5 or 6 and those colonies grew very slowly, and the numbers of tumors recovered remained substantially lower in comparison to those which did not receive NK injection (FIGS. 18C-18I). Similarly, NK-differentiated tumors when implanted in mice and their pancreas were dissociated after sacrifice, tumors did not grow or grew very few colonies at later days and their growth remained extremely slow (FIGS. 16B, 18E, and 18H), however, blocking differentiation with anti-IFN-γ and anti-TNF-α antibodies allowed attachment and growth of the tumors at 24-48 hours with an increased kinetics of growth (FIG. 18H). Tumor growth after dissociation and plating was slightly less in mice fed with AJ2 and injected with NK cells in comparison to NK alone injected mice, and both were substantially less than those which only received implantation of the MP2 tumors (FIGS. 18D, 18F, 18G, and 18I). There was 18-22 fold more infiltrating huCD45+ immune cells in pancreas cultured from mice injected with tumors and NK cells in comparison to tumor-alone injected mice (FIGS. 19E and 18J). Greater percentages of infiltrating huCD45+ immune cells within the pancreas of NK injected tumor-bearing mice expressed CD94, and NKG2D surface receptors, whereas they expressed similar percentages of DNAM surface receptors when compared to tumor-bearing mice in the absence of NK injection (FIGS. 18J and 18K).

On average a decrease in secreted IFN-γ from the pancreatic cell cultures could be observed in mice implanted with MP2 tumors, as compared to control mice with no tumors (FIGS. 19F and 18L). Injection of NK cells to tumor-bearing mice restored IFN-γ secretion in pancreatic cell cultures and the levels exceeded those seen in the control mice with no tumors (FIGS. 19F and 18L). Implantation of NK-differentiated MP2 tumors did not result in inhibition of IFN-γ in pancreatic cell cultures and the amounts were comparable to those obtained from control mice with no tumors (FIG. 19F). In contrast, IL-6 secretions were the highest in pancreatic cell cultures from tumor-bearing mice, and it was substantially lower in all other groups of mice (FIG. 19G). Although no significant differences could be seen between NK alone injected or NK-injected and AJ2 fed mice in terms of tumor weight/tumor growth, but on average there were differences in IFN-γ release between these groups (FIG. 18L).

The expression of B7H1 (PD-L1), MHC-class I and CD54 were higher on MP2 tumors cultured from the pancreas of NK-injected mice when compared to tumor-bearing mice in the absence of NK injection (FIG. 19H). Moreover, similar to in vitro experiments, cultured MP2 tumors from NK-injected mice exhibited decreased susceptibility to NK cell-mediated killing, whereas tumors cultured from tumor-bearing mice in the absence of NK injection remained significantly more susceptible (FIGS. 19I and 19J). When NK cell mediated differentiation of tumors was blocked with antibodies against IFN-γ and TNF-α, susceptibility to NK cell-mediated cytotoxicity was restored (FIG. 19J).

### Example 21: Suppression of NK Cell Cytotoxicity and Decreased Secretion of IFN-γ in Tumor-Bearing Mice within all Tissue Compartments, and their Restoration/Increase with the Injection of NK Cells in the Presence and Absence of Feeding with AJ2

PBMCs from tumor-bearing mice (FIG. 20A) similar to PBMCs (FIG. 21A) and NK cells (FIG. 21B) from pancreatic cancer patients, had significantly lower NK cell mediated cytotoxicity and exhibited decreased IFN-γ secretion when compared to those from healthy mice or humans respectively (FIGS. 20B, 20C, and 21C-21F). When splenocytes (FIGS. 20D-20F, and 21G-21H), enriched-NK cells from splenocytes (FIGS. 20G-20H, and 21I), CD3+ T cells from splenocytes (FIGS. 201 and 21J) and BM-derived immune cells (FIGS. 20J-20L, and 21K-21L) were assessed for NK cytotoxicity and/or secretion of IFN-γ, tumor-bearing mice had much lower cytotoxicity and/or secretion of IFN-γ in cells obtained from all tissue compartments, in comparison to those obtained from control mice without tumor, or tumor-bearing mice injected with NK cells, or those implanted with NK-differentiated tumors (FIGS. 20 and 21). Blocking NK differentiation of the tumors by anti-IFN-γ and anti-TNF-α antibodies resulted in a similar magnitude of IFN-γ secretion to those obtained from undifferentiated tumors in all tissue compartments tested. (FIGS. 20C, 20F, 20L, 21F, 21H, and 21L).

Similar to those seen with the pancreatic tumors, implantation of oral tumors in the oral cavity of hu-BLT mice resulted in similar profiles of cytotoxicity and secretion of IFN-γ from PBMCs isolated from oral tumor bearing mice in the presence and absence of NK injection and feeding with AJ2 (FIG. 22). PBMCs isolated from tumor-bearing mice had significantly less cytotoxicity and secretion of IFN-γ when compared to either control mice in the absence of tumor implantation or NK injected tumor-bearing mice; and the highest increase was seen in tumor-bearing mice injected with NK cells and fed with AJ2 (FIGS. 22A and 22B). Tumor-bearing mice fed with AJ2 had on average an increase in cytotoxicity and in fold increase in IFN-γ secretion when compared to tumor-bearing mice in the absence of AJ2 feeding but the levels were much less than those seen from tumor-bearing mice injected with NK and/or fed with AJ2 (FIGS. 22A and 22B). Feeding AJ2 to tumor-bearing mice in the absence of NK injection improved cytotoxicity and secretion of IFN-g moderately (FIG. 22).

Since NK mediated inhibition of tumor growth in the pancreas was very strong, no tumor growth either in NK injected tumor-bearing mice or those receiving both NK and anti-PD1 antibody can be seen when compared to tumor-bearing mice (FIGS. 23A and 23B). However, IV injection of anti-PD1 in combination with NK cells elevated secretion of IFN-γ in all tissue compartments tested with the exception of cells dissociated from pancreas in which a slight increase can be seen in comparison to NK alone injected mice (FIGS. 23C-23H). Anti-PD1 antibody injection in the absence of NK cells in tumor-bearing mice increased secretion of IFN-γ in all tissues with the exception of cells dissociated from pancreas when compared to those from tumor alone injected mice (FIGS. 23C-23H).

### Example 22: Paclitaxel Induce Significant Cell Death in NK-Differentiated MP2 Tumors Treated with/without N-Acetylcysteine (NAC)

Unlike MP2 tumors, treatment of well-differentiated PL12 and Capan tumors with paclitaxel (FIGS. 24 and 25A) demonstrated higher levels of cell death. Similarly, differentiation of MP2 tumors with NK-supernatants resulted in susceptibility to paclitaxel (FIG. 25B) mediated cell death and blocking NK-supernatant mediated differentiation with anti-IFN-γ and anti-TNF-α antibodies substantially decreased the cell death induced by paclitaxel (FIG. 25B). As shown in (FIGS. 24 and 25A), the addition of NAC to MP2, PL12 and Capan increased paclitaxel mediated cell death. Similarly, the addition of NAC to NK-supernatant differentiated MP2 tumors increased cell death and blocking differentiation with IFN-γ and TNF-α mAbs decreased paclitaxel mediated cell death (FIG. 25B). The differentiation potential of cells by NAC was shown before, addition of paclitaxel or cis-dichlorodiammineplatinum (CDDP or Cisplatin) to patient-derived differentiated OSCCs or NK-differentiated OSCSCs also mediated higher cell death, whereas minimal effects were seen on stem-like/poorly differentiated OSCSCs.

### Example 23: Monocytes or Osteoclasts from Tumor-Bearing Mice Injected with NK Cells or from NK-Differentiated MP2-Implanted Tumors had Higher Capacity to Activate NK Cells when Compared to Those of Tumor-Alone Implanted Mice

Purified NK cells from different groups of mice were cultured with their respective autologous monocytes (FIGS. 26A and 27A), or allogeneic NK cells purified from healthy human donors were cultured with osteoclasts derived from monocytes isolated from BM of each of the groups of mice as shown in FIGS. 26B-26D, and the levels of NK expansion, cytotoxicity, and IFN-γ secretion by the NK cells were assessed. NK cells cultured with autologous monocytes from tumor-bearing mice injected with the NK cells or those implanted with NK-differentiated MP2 tumors had much higher ability to induce IFN-γ secretion when compared to those of tumor-bearing mice in the absence of NK injection (FIGS. 26A and 27A). Allogeneic NK cells cultured with osteoclasts from mice implanted with tumor and injected with NK cells or implanted with NK-differentiated tumors had significantly higher expansion and function as compared to those from tumor-bearing mice in the absence of NK injection (FIGS. 26B-26D). Similar results were obtained when osteoclasts from pancreatic-cancer patients were cultured with allogeneic healthy human NK cells when compared to those cultured from healthy individuals (FIGS. 27B-27D). Osteoclasts from cancer patients were less able to expand NK cells (FIG. 27B) or increase NK cell mediated cytotoxicity (FIG. 27C) or NK cell mediated secretion of IFN-γ (FIG. 27D) when compared to those from healthy donors. When examining the surface receptor expression on cancer-patient and healthy individuals' osteoclasts, decreased expression of MHC-class I, CD54, KLRG1, KIR2/KIR3 and MICAS could be seen on cancer patients' OCs as compared to healthy OCs (FIG. 27E).

Finally, when the same amount of IFN-γ from the supernatants of NK cells were used to differentiate OSCSC tumors, those from pancreatic cancer patients' NK cells were less effective in differentiating OSCSC tumors as compared to those from healthy donors' NK cells. (FIGS. 28A-28B). NK supernatants from patients elevated MHC-class I moderately and induced only 35% resistance of OSCSC tumors to NK-mediated cytotoxicity, whereas NK supernatants from healthy individuals elevated MHC-class I substantially and induced 78% resistance of OSCSCs against NK-mediated cytotoxicity (FIGS. 28A-28B). Supernatants from both patients' and healthy individuals' T cells differentiated OSCSCs, albeit patient supernatants were still inferior to those from healthy individuals' T cells (FIG. 28C). The rationale for using OSCSCs is because these cells are highly sensitive to IFN-γ mediated differentiation.

NK cells limit growth and expansion of CSCs/poorly differentiated pancreatic tumors by tumor lysis and differentiation. MP2 tumors being stem-like/poorly differentiated, form large tumors in NSG and hu-BLT mice, and they have the ability to metastasize to other organs/tissues, whereas their NK-differentiated MP2 tumors or patient-derived well-differentiated PL-12 tumors form no or very small tumors respectively in the pancreas without metastatic potential. Indeed, the growth potential of MP2 tumors in vitro is found to be 10-15 fold, whereas those of the NK-differentiated counterparts are between 1.5-4 fold when the same numbers of tumors are cultured at the same time period, and no or slight cell death could be seen in the cultures of either undifferentiated MP2 tumors or those differentiated by the NK cells.

Patient-derived PL12 tumors or NK-differentiated tumors, although were not killed by primary NK cells, they were however, susceptible to chemo-drugs and were killed by paclitaxel (FIGS. 24 and 25) as well as CDDP, whereas stem-like/poorly-differentiated tumors were relatively resistant. Indeed, when NK-induced differentiation of MP2 tumors was inhibited by the combination of anti-IFN-γ and anti-TNF-α antibodies, tumors lost sensitivity to chemotherapy drugs and they became susceptible to NK cell mediated cytotoxicity. Moreover, NAC which is known to differentiate cells in addition to its other effects increased paclitaxel mediated cell death in NK-differentiated MP2 tumors and in patient-derived well differentiated pancreatic tumors (FIGS. 24 and 25B).

Hu-BLT monocyte derived osteoclasts expanded hu-BLT NK cells similar to human NK cells expanded by autologous osteoclasts. In addition, both autologous and allogeneic osteoclasts were able to expand hu-BLT NK cells with hu-BLT osteoclasts having slightly higher NK expansion potential (FIG. 29B). Hu-BLT NK cells (FIG. 29D) expanded by hu-BLT or human derived osteoclasts secreted IFN-γ (FIG. 29E). Such similarities in NK responses between hu-BLT and human-derived NK cells in their capabilities to expand by the autologous osteoclasts and secrete higher levels of IFN-γ and mediate increased cytotoxicity provides partly the rationale for the use of this animal model as a surrogate model for the studies of human disease. Furthermore, the hu-BLT mice model is an appropriate model to study mechanisms underlying NK defect and cancer progression since similar defects in both tumor-bearing hu-BLT and cancer patients' NK cells is found.

Similar in NSG mice, NK-differentiated MP2 tumors did not grow to the levels which could form visible tumors in hu-BLT mice, and when tumor differentiation was prevented using antibodies to IFN-γ and TNF-α, tumors grew substantially (FIGS. 18C, 18H, and 19D). Injection of autologous or allogeneic NK cells in the presence or absence of AJ2 feeding resulted in a significant inhibition of tumor growth in hu-BLT mice, consistent with significant increase in NK function by probiotic bacteria.

When the pancreas was dissociated and cultured from tumor-bearing mice injected with NK cells and fed with/without AJ2, very few tumors grew and those which grew were of differentiated phenotype, whereas tumors from tumor-bearing mice in the absence of NK cell injection grew rapidly and remained undifferentiated. Moreover, tumors dissociated and cultured from NK-injected tumor-bearing hu-BLT mice contained about 18-22 fold more huCD45+ immune cells when compared to those cultured from dissociated tumors from tumor-bearing mice in the absence of NK injection. In addition, there were substantial increases in IFN-γ secretion but much lower IL-6 secretion in pancreatic cell cultures from tumor-bearing mice injected with NK cells, and the highest increases in IFN-γ secretion were seen in tumor-bearing mice fed with AJ2 and injected with NK cells, whereas tumor bearing mice in the absence of NK injection had higher secretion of IL-6 in the presence of lower IFN-γ secretion from pancreatic cell cultures. Increased IL-6 secretion is likely due to the growing tumors in tumor-bearing mice. NK cells in different tissues of hu-BLT mice implanted with tumor and injected with NK cells in the presence/absence of feeding with AJ2 mediated significant cytotoxicity and secreted increased amounts of IFN-γ, whereas those from tumor-bearing mice in the absence of NK injection mediated much less cytotoxicity or IFN-γ secretion.

The single injection of super-charged NK cells to tumor-bearing mice resulted in increased surface receptor expression of PD-L1, CD54 and MHC-class I on implanted tumor cells, decreased growth, and mediated loss of susceptibility of tumor cells to NK cell-mediated cytotoxicity (FIGS. 19H-19J), thereby paving the road for the increased susceptibility to cytotoxic T lymphocyte (CTL) mediated killing due to increased MHC-class I expression. Greater percentages of NKT cells were also seen in tumors resected from NK injected tumor-bearing mice as compared to tumor implanted mice in the absence of NK injection. Increased percentages of T cells in tumor-bearing mice in the presence of decreased percentages of NK cells in the pancreas could be problematic for successful removal of stem like/undifferentiated tumors since these tumors will not be targeted efficiently by the NK cells in the pancreas leading to tumor persistence and expansion.

Similar to cancer patients' monocytes and osteoclasts, those from tumor-bearing mice had much lower ability to expand autologous or allogeneic NK cells or increase their functional potential. More severe inhibition of NK cell expansion and function is seen when both NK and monocytes are from tumor-bearing mice due to the combined defects in both NK cells and monocytes. These experiments not only highlight similarities between the tumor-bearing hu-BLT mouse model and human cancers but also indicate a severe deficiency in the function of NK cell activating effectors in tumor-bearing hu-BLT mice similar to cancer patients. It is also important to note that the highest activation of NK cells from hu-BLT mice was achieved by implantation of NK-differentiated tumors, suggesting that optimal differentiation of tumors can indeed promote and maintain intact monocyte/osteoclast function.

To understand underlying mechanisms which govern inhibition of NK cell function, the surface expression of osteoclasts was determined from cancer patients in comparison to healthy donors' osteoclasts. The findings indicated that not only inhibitory MHC-class I expression is down-regulated but also activating CD54, KLRG1 and MICAS surface expressions were decreased, suggesting an overall decrease in NK ligand expression. Loss of activating ligands could clearly be a reason for decreased activation of NK cells. However, loss of inhibitory receptors provides a more complex picture. Loss of expression of activating and inhibitory NK cell ligands was also seen on osteoclasts from KC mice with pancreatic KRAS mutation correlating with the loss of NK cell function and generation of pancreatic tumors.

Supernatants from patient's NK cells were less able to differentiate tumors indicating that the function of secreted IFN-γ from patient NK cells is also severely compromised. Thus, pancreatic tumor induction and progression in patients is due to not only combined defects in NK expansion, decreased NK-cell mediated cytotoxicity and lower secretion of IFN-γ, and much lower ability of secreted IFN-γ to differentiate tumors but also due to the defects in other subsets of immune cells which support NK cell expansion and function.

### Example 24: Materials and Methods for Examples 25-32

Sera Collection from Human and Hu BLT Mice Peripheral Blood

Peripheral blood (200 μl) was collected in 1.5 ml eppendorf with no heparin was left in room temperature for 15-20 minutes before it was centrifuged at 2000 rpm for 10 mins, sera layer was then harvested.

Human Single-Color Enzymatic ELISPOT Assay for IFN-γ 80 μl of anti-human IFN-γ capture antibody was added to each well of a 96-well high-protein-binding PVDF filter plate and incubated overnight at 4° C. The plate was washed with 150 μl of PBS once before adding samples into the plate. 50,000 cells in 200 μl of RPMI were added into each well and incubate at 37° C., 5% CO₂ overnight. After incubation, the plate was washed twice with 200 μl PBS followed by 0.05% 200 μl Tween-PBS twice. 80 μl of anti-human IFN-γ detection antibody was added into each well and incubated at room temperature for 2 hours and the plate was washed three times with 200 μl/well of 0.05% Tween-PBS. 80 μl/well of tertiary solution which was made from 1:1000 diluted Strep-AP was added in the plate and incubated for 30 minutes. The plate was washed twice with 200 μl/well of 0.05% Tween-PBS followed by 200 μl/well distilled water twice. Then, 80 μl/well of blue development solution was added, and the plate was incubated at room temperature for 15 minutes. The reaction was stopped by gently rinsing membrane with tap water for 3 times. Air-dried the plate for 2 hours and was scanned to count IFN-γ release using CTL machine with immunoSpot® Software. (Cellular Technology Limited, OH, USA).

### Cell Lines, Reagents, and Antibodies

RPMI 1640 (Life Technologies, CA) supplemented with 10% fetal bovine serum (FBS) (Gemini Bio-Product) was used to culture human NK cells, human T cells and hu-BLT mice immune cells. Oral squamous carcinoma stem cells (OSCSCs) were isolated from patients with tongue tumors at UCLA, RPMI 1640 supplemented with 10% FBS was used for the OSCSCs cultures. Alpha-MEM (Life Technologies, CA) with 10% FBS was used for osteoclast and dendritic cell cultures. M-CSF, anti-CD16mAb and flow cytometric antibodies were purchased from Biolegend, CA. RANKL, GM-CSF and IL-4 were purchased from PeproTech, NJ, and recombinant human IL-2 was obtained from NIH-BRB. Human anti-CD3 was purchased from Stem Cell Technologies. Propidium iodide (PI) was purchased from Sigma, MO.

### Purification of Human NK Cells and T Cells

Written informed consents approved by UCLA Institutional Review Board (IRB) were obtained from healthy donors and cancer patients and all procedures were approved by the UCLA-IRB. NK cells, CD3+ T cells, CD4+ T cells, and CD8+ T were isolated from PBMCs using the EasySep® Human NK cell enrichment kit, EasySep® Human T cell enrichment kit, EasySep® Human CD4 T cell enrichment kit, and EasySep® Human CD8 T cell enrichment kit respectively purchased from Stem Cell Technologies (Vancouver, BC, Canada). Isolated NK cells and T cells were stained with anti-CD16, anti-CD3, anti-CD4 and anti-CD8 to measure the cell purity using flow cytometric analysis.

Purification of Human Monocytes and, Generation of Osteoclasts and Dendritic Cells

Monocytes were negatively selected and isolated from PBMCs using the EasySep® Human monocyte isolation kit purchased from Stem Cell Technologies (Vancouver, BC, Canada). Isolated monocytes were stained with anti-CD14 antibody to measure the cell purity using flow cytometric analysis, greater than 95% purity was achieved. Monocytes were differentiated to osteoclasts by treating with M-CSF (25 ng/mL) and RANKL (25 ng/mL) for 21 days. To generate dendritic cells (DCs), monocytes were treated with GM-CSF (150 ng/mL) and IL-4 (50 ng/mL) for 7 days.

### Probiotic Bacteria (AJ2)

AJ2 is a combination of 8 different strains of gram-positive probiotic bacteria (Streptococcus thermophiles, Bifidobacterium longum, Bifidobacterium breve, Bifidobacterium infantis, Lactobacillus acidophilus, Lactobacillus plantarum, Lactobacillus casei, and Lactobacillus bulgaricus) are selected for their superior ability to induce optimal secretion of both pro-inflammatory and anti-inflammatory cytokines in NK cells [56]. For sonication, AJ2 bacteria were weighed and re-suspended in RPMI 1640 medium containing 10% FBS at a concentration of 10 mg/ml. The bacteria were thoroughly vortexed, then sonicated on ice for 15 seconds, at 6 to 8 amplitudes. Sonicated samples were then incubated for 30 seconds on ice. After every five pulses, a sample was taken to observe under the microscope until at least 80 percent of bacteria walls were lysed. It was determined that approximated 20 rounds of sonication/incubation on ice, were conducted to achieve complete sonication. Finally, the sonicated AJ2 (sAJ2) were aliquoted and stored in a −80 degrees Celsius until use.

### Expansion of Human NK Cells and Human T Cells

Human purified NK cells were activated with rh-IL-2 (1000 U/ml) and anti-CD16mAb (3 μg/ml) for 18-20 hours before they were co-cultured with feeder cells (osteoclasts or dendritic cells) and sAJ2 (NK:OCs or DCs:sAJ2; 2:1:4). The medium was refreshed every 3 days with RMPI containing rh-IL-2 (1500 U/ml). Human purified T cells were activated with rh-IL-2 (100 U/ml) and anti-CD3 (1 μg/ml) for 18-20 hours before they were co-cultured with/without osteoclasts and with/without sAJ2 (T:OCs:sAJ2; 2:1:4). The culture media was refreshed with rh-IL-2 (150 U/ml) every three days.

### Tumor Implantation in Hu-BLT Mice

Animal research was performed under the written approval of the UCLA Animal Research Committee (ARC) in accordance to all federal, state, and local guidelines. Combined immunodeficient NOD.CB17-Prkdcscid/J and NOD.Cg-Prkdcscid Il2rgtm1Wj1/SzJ (NSG lacking T, B, and natural killer cells) were purchased from Jackson Laboratory. Humanized-BLT (hu-BLT; human bone marrow/liver/thymus) mice were prepared on NSG background as described previously. To establish orthotopic tumors, mice were first anesthetized with isoflurane in combination with oxygen, and human OSCSCs tumor cells were then directly injected in the floor of mouth in suspension with 10 μl HC Matrigel (Corning, N.Y., USA) (1×106 cells). Four to five weeks after the tumor injections, mice were euthanized, and bone marrow, spleen, and peripheral blood were harvested.

Cell Isolations from Hu-BLT Mice BM, Spleen and Peripheral Blood

To obtain single-cell suspensions from BM, femurs were cut from both ends and were flushed from one end to other using RPMI 1640 media, afterward BM cells was filtered through a 40 μm cell strainer. To obtain single-cell suspensions from spleen, spleen was smashed until no big piece was left and sample was filtered through a 40 μm cell strainer and centrifuged at 1500 rpm for 5 minutes at 4° C. The pellet was re-suspended in ACK buffer to remove the red blood cells for 2-5 mins followed re-suspension in RMPI media and centrifuged at 1500 rpm for 5 minutes at 4° C. Peripheral blood mononuclear cells (PBMCs) were isolated using ficoll-hypaque centrifugation of heparinized blood specimens. The buffy coat containing PBMCs were harvested, washed and re-suspended in RPMI 1640 medium.

### ELISA and Multiplex Cytokine Array Kit

Single ELISAs and multiplex assays were performed as described previously. To analyze and obtain the cytokine and chemokine concentration, a standard curve was generated by either two or three-fold dilution of recombinant cytokines provided by the manufacturer. For multiple cytokine array, the levels of cytokines and chemokines were examined by multiplex assay, which was conducted as described in the manufacturer's protocol for each specified kit. Analysis was performed using a Luminex multiplex instrument (MAGPIX, Millipore, Billerica, Mass.) and data was analyzed using the proprietary software (xPONENT 4.2, Millipore, Billerica, Mass.).

### Surface Staining Assay

For surface staining, the cells were washed twice using ice-cold PBS+1% BSA (Bovine serum albumin). Predetermined optimal concentrations of specific human monoclonal antibodies were added to 1×10⁴ cells in 50 μl of cold PBS+1% BSA and cells were incubated on ice for 30 min. Thereafter cells were washed in cold PBS+1% BSA and brought to 500 μl with PBS+1% BSA. Flow cytometric analysis was performed using Beckman Coulter Epics XL cytometer (Brea, Calif.) and results were analyzed in FlowJo vX software (Ashland, Oreg.).

### ⁵¹Cr Release Cytotoxicity Assay

The ⁵¹Cr release assay was performed as described previously. Briefly, different numbers of effector cells were incubated with ⁵¹Cr-labeled target cells. After a 4-hour incubation period the supernatants were harvested from each sample and counted for released radioactivity using the gamma counter. The percentage specific cytotoxicity was calculated as follows:

$$\% Cytotoxicity = \frac{Experimental cpm - spontaneous cpm}{Total cpm - spontaneous cpm}$$

LU 30/106 is calculated by using the inverse of the number of effector cells needed to lyse 30% of tumor target cells×100.

### Target Cell Visualization Assay (TVA)

Target cells were incubated with TVATM dye at 370 C for 15 mins, afterwards effector cells were cultured with target cells for 4 hours. After a 4-hour incubation period the target cells were counted with immunospot at 525 nm emission wavelengths. The percentage specific cytotoxicity was calculated as follows:

$$\% Cytotoxicity = \frac{Experimental cpm - spontaneous cpm}{Total cpm - spontaneous cpm}$$

LU 30/107 is calculated by using the inverse of the number of effector cells needed to lyse 30% of tumor target cells×100.

### Statistical Analysis

The prism-7 software is used for the statistical analysis. An unpaired or paired, two-tailed student t-test was performed for the statistical analysis. One-way ANOVA with a Bonferroni post-test was used to compare different groups. (n) denotes the number of human donors or mice. For in-vitro studies either duplicate or triplicate samples were used for assessment. The following symbols represent the levels of statistical significance within each analysis, ***(p value<0.001), **(p value 0.001-0.01), *(p value 0.01-0.05).

### Example 25: Suppression of NK Cell-Mediated Cytotoxicity and/or Secretion of Cytokines in Cancer Patients

Lower numbers of PBMCs were recovered from the peripheral blood of cancer patients when compared to those isolated from healthy individuals (FIG. 30A). Higher percentages of CD16+CD56+, CD14+, CD11b+ cells, and low percentages of CD3+, and CD19+ cells were obtained within CD45+ PBMCs isolated from cancer patients in comparison to healthy individuals (FIG. 30B). NK cells from cancer patients exhibited decreased IFN-γ secretion (FIGS. 30C and 30E) and, significantly lower NK cell mediated cytotoxicity when compared to NK cells from healthy individuals (FIG. 30D). In addition, similar to IFN-γ, secretion of IL-12p70, IL-6, TNF-α, IL-5, and IL-4 were also significantly lower from cancer patients' NK cells when compared to those from healthy individuals (FIG. 30E). Similar results were seen in the sera collected from peripheral blood of the cancer patients (FIG. 31A).

### Example 26: Unlike T Cells, Significant Suppression of IFN-γ Secretion was Seen from Cancer Patients' NK Cells Both in the Absence or Presence of OCs

Purified NK cells from cancer patients and healthy individuals were cultured with healthy allogeneic OCs, and the levels of NK cell expansion, cytotoxicity and IFN-γ secretion were assessed. NK cells from cancer patients had significantly lower expansion (FIG. 32A), and lower NK cell-mediated cytotoxicity (FIG. 32B) compared to those from healthy individuals (FIGS. 32A and 32B). Cancer patients' NK cells also mediated significantly lower levels of IFN-γ secretion both in the absence or presence of OCs (FIGS. 32C, 32D, and 33A-33C). Similar to NK cells, T cells from cancer patients had significantly lower expansion rate both in the absence or presence of OCs (FIGS. 32E and 34A). However, in contrast to NK cells, cancer patients' T cells had a slight decrease in IFN-γ secretion both in the absence or presence of OCs (FIGS. 32F-32G and 34B-34F), even though on per cell basis only IL-2 activated T cells from cancer patients had significantly lower levels of IFN-γ secretion (FIGS. 32G and 34C). Next, we compared the expansion profile and IFN-γ secretion of NK cells and T cells of healthy individuals in the presence of OCs (FIGS. 32H, 32I, and 34G). There was a significantly higher expansion of T cells in comparison to NK cells in the absence of OCs (FIGS. 32H and 34G). However, OCs induced 1.2 to 1.6 fold higher T cell expansion when compared to cultures without the OCs, whereas OCs induced 2.6-4.5 fold expansion in NK cells (FIGS. 32H and 34G). These results indicated that OCs induce higher expansions of NK cells than T cells.

### Example 27: Cancer Patients' T Cells Exhibit Effector Memory Surface Phenotype, Decreased CD4/CD8 T Cells Ratio; CD8+ T Cells were Further Increased in the Presence of OCs

Higher percentages of CD45RO expressing T cells in the presence of lower percentages of CD45RA were observed in PBMCs isolated from cancer patients, whereas T cells from healthy individuals exhibited the inverse relationship CD45RA>CD45RO (FIG. 35A). Moreover, the percentages of T cells expressing CD62L, CD28, CCR7, and CD127 were lower in cancer patients than in healthy individuals (FIG. 35A). In addition, the percentages of T cells expressing both CD28 and CD127 were lower in cancer patients than in healthy individuals (FIG. 35A). Moreover, cancer patients' T cells have increased percentages of CD8+ T cells and decreased CD4+ T cells in comparison to those isolated from healthy individuals, resulting in decreased CD4/CD8 ratios (FIGS. 35B and 35C).

Next, purified NK cells and T cells were each cultured with OCs and determined the fractions of CD4+ and CD8+ T cells within both the NK and T cell co-cultures with OCs. Purified T cells cultured with OCs increased the percentages of CD8+ T cells and the ratio of CD4/CD8 decreased from 2.4 in T cells in the absence of OCs to 1.2 in those cultured with OCs (FIG. 35D). In contrast, T cells expanded within NK cultures with OCs significantly increased the percentages of CD8+ T cells and accordingly the ratio of CD4/CD8 decreased substantially (FIG. 35E). Similar trends were also seen when T cells and NK cells from the patients were cultured with OCs, except T cells cultured with OCs from patients had lower CD4/CD8 ratios when compared to healthy controls (FIG. 35E). Thus, purified T cells isolated from healthy individuals and cancer patients in the absence of NK cells failed to expand CD8+ T cells significantly, although cancer patients' T cells had higher percentages of CD8+ T cells constitutively (FIGS. 35D and 35E). In addition, purified NK cells activated with OCs which contained undetectable or negligible levels of contaminating T cells at the start of the culture, expanded CD8+ T cells at the later times during the expansion period from both healthy and patient cultures, albeit patient NK cell cultures expanded CD8+ T cells faster than healthy NK cells (FIGS. 35D and 35E).

### Example 28: Osteoclast Expanded NK Cells Secrete More Cytokine and Chemokines when Compared to Contaminate T Cells in the Same Culture

Higher secretion of cytokines, chemokines, soluble Fas-Ligand and perforin except GM-CSF and IL-13 were seen in OCs expanded NK cells compared to CD3+ T, CD4+ T or CD8+ T cells those purified from day 12 OCs expanded NK cells culture (FIGS. 37A and 37B). CD4+ T secreted least soluble Fas-Ligand, whereas CD8+ T secreted least MIP-la and MIP-1b (FIGS. 37A and 37B). The secretion levels of GM-CSF, soluble CD137, IFN-γ, soluble Fas, soluble Fas-Ligand, perforin, MIP-1a and MIP1b were higher in OCs expanded NK cells when compared to OCs expanded T cells (FIGS. 37C-37F). The secretion levels of IL-10, granzyme A and B, and TNF-α were seen lower in OCs expanded NK cells when compared to OCs expanded T cells (FIGS. 37C-37F). Next, the secreted factors were examined from CD8+ T cells isolated from day 12 OCs expanded NK cells culture and CD8+ T cells isolated from day 12 OCs expanded CD8+ T culture. The CD8+ T isolated from OCs expanded NK cells culture showed higher secretion levels of GM-CSF, soluble CD137, IFN-γ, IL-10, soluble Fas-Ligand and TNF-α, lower levels of granzyme A and perforin, whereas similar levels of granzyme B and soluble Fas when compared to CD8+ T cells isolated form OCs expanded CD8+ T cells (FIG. 37G).

### Example 29: T Cells in the OC-Expanded NK Cells Preferentially Expanded CD8+ T Cells Whereas DC-Expanded NK Cells Generated CD4+ T Cells

When purified NK cells from healthy individuals with no/few T cells contaminants were cultured with either OCs or DCs, there was significantly higher NK cell expansion in the presence of OCs (FIG. 36A). When the numbers of NK and T cells were determined based on CD16 and CD3 surface expressions respectively, there were significantly higher numbers of NK cells (FIG. 36B) and lower numbers of T cells (FIG. 36C) in the presence of OCs when compared to those in the presence of DCs. OC-expanded lymphocytes mediated more cytotoxicity against OSCSCs when compared to those expanded by DCs per NK cell basis (FIG. 36D). When adjusted based on the numbers of NK cells those which were expanded by OCs had higher cytotoxicity as compared to DC-expanded NK cells (FIG. 36E). OC-expanded lymphocytes secrete more IFN-γ when compared to those expanded by DCs (FIG. 36F). Next, the subpopulations of T cells were determined within the expanded NK cells cultured by OC vs. DCs, in which DCs preferentially induced the expansion of CD4+ T cells (FIGS. 36G, 36I, 36J and 36M) whereas OCs induced the expansion of CD8+ T cells (FIGS. 36H-36J and 36M). The T cells within the NK cell cultures expanded with DCs expressed slightly higher levels of KLGR1 and TIM3, whereas PD1 expression levels were similar on DC expanded T cells when compared to OC expanded T cells (FIG. 36J). Purified T cells cultured with either OCs or DCs, expressed slight differences in the levels of CD4, CD8, KLRG1 and TIM3 and PD-1 expression (FIG. 36K). T cells within OC-expanded NK cells culture exhibited higher expression of CD45RO, but much lower levels of CD62L, CD28, CCR7 and CD127 and, similar levels of CD44 when compared to those expanded in DCs-expanded NK cells cultures (FIGS. 36K and 36L). Purified T cells expanded in the presence of OCs expressed slightly higher expression of CD45RO and CD28, but expressions of CD62L and CCR7 were slightly low with very similar expression of CD127 and CD44 when compared to those expanded in the presence of DCs (FIGS. 36L and 36M). Osteoclast-expanded NK cells secrete more cytokines and chemokines when compared to expanded T cells in the same culture (FIG. 37).

### Example 30: Immunotherapy with NK Cells Increased CD8+ T Cells and, Resulted in an Increase in IFN-γ Secretion and NK Cell-Mediated Cytotoxicity in Oral Tumor-Bearing Hu-BLT Mice

Hu-BLT mice were implanted with OSCSCs in the oral cavity and injected with super-charged NK cells with potent cytotoxic and cytokine secretion capabilities. After several weeks, mice were sacrificed and tissues were removed, dissociated and the cells were analyzed (FIG. 38A). Increased proportions of CD3+CD8+ T cells within BM (FIG. 38B), spleen (FIG. 38E) and peripheral blood (FIG. 38H) were seen in NK-injected tumor-bearing mice as compared to the other groups. Injection of NK cells resulted in an increased IFN-γ secretion from BM (FIG. 38C), spleen (FIG. 38F) and peripheral blood (FIG. 38I), and increased NK cell-mediated cytotoxicity in BM (FIG. 38D), spleen (FIG. 38F), and peripheral blood (FIG. 38J) in tumor-bearing mice when compared to those in the absence of NK cell injection. Interestingly, sera from peripheral blood of NK-injected tumor-bearing mice exhibited increased IFN-γ, IL-6 and ITAC, but decreased IL-8 and GM-CSF in NK-injected tumor-bearing mice when compared to tumor-bearing mice in the absence of NK cells injection (FIG. 39).

### Example 31: Higher Expansion and Increased IFN-γ Secretion from OC-Expanded CD8+ T Cells when Compared to OC-Expanded CD4+ T

The purified CD4+ T cells and CD8+ T cells were treated with anti-CD3/CD28 in the presence of IL-2 to assess the degree of expansion (FIG. 40A). No significant differences could be seen between CD4 and CD8 T cells activated with anti-CD3 and anti-CD28 in the absence of OCs (FIG. 40A). However, when purified CD4+ T cells and CD8+ T cells treated with anti-CD3/CD28 in the presence of IL-2 were cultured with OCs and sAJ2, although initially both expanded equal rates, after day 6 CD8+ T cells continued to expand and increase their fold expansion, whereas CD4+ T cells remained steady and after day 12 it started decreasing their fold expansion, even though it was still expanding but at a much lower rates than CD8+ T cells (FIG. 40B). Purified NK cells, CD4+ T cells, and CD8+ T cells were treated as described in FIG. 6B and cultured in the presence or absence of OCs and fold expansion in each cell type were compared to those without OCs. As shown is FIG. 6C, both NK cells and CD8+ T cells expanded significantly more when compared to CD4+ T cells in the presence of OCs (FIG. 40C). Similarly, both NK cells and CD8+ T cells secreted significantly higher IFN-γ when compared to CD4+ T cells in the presence of OCs per cell basis (FIG. 40D).

When the levels of cytokines and chemokines were assessed in OC expanded NK cells and compared to OC expanded T cells, NK cells secreted higher levels of cytokines and chemokines (FIG. 37). In particular, NK cells had greater than 30 fold higher MIP-la and MIP-1B, and greater than 10 fold for sCD137 and Fas ligand and greater than four fold GMCSF and IFN-γ, and greater than 2 fold for sFas and perforin when compared to T cells on per cell basis (FIGS. 37D and 37F). The secretion of the cytokines was not contributed by the T cells contaminating the NK cells since the assessments were made on day 6 which no expansion of T cells could be seen (FIGS. 37A-37F). CD8+ T cells sorted out from OC expanded NK cells at day 12 of expansion secreted higher levels of GMCSF, sCD137, IFN-γ, Fas Ligand, IL-10 and TNF-α when compared to OC expanded CD8+ T cells under the same activation conditions in the absence of NK cells (FIG. 37G).

### Example 32: NK Cells Preferentially Lyse CD4+ T Cells and not CD8+ T Cells

CD4+ and CD8+ T cells were positively selected and they were further activated with anti-CD3 and IL-2 (FIGS. 40E and 41), in another set CD4+ and CD8+ T cells were positively selected and they were only activated with IL-2 (FIG. 40F) before T cells were subjected to NK cell mediated cytotoxicity assay using TVA. NK cells preferentially lysed CD4+ T cells and not CD8+ T cells (FIGS. 40E, 40F, and 41).

NK functional inactivation and loss of numbers occurs at the pre-neoplastic stage of pancreatic cancer due to the effects of both the KRAS mutation and high fat calorie diet. It is demonstrated herein that patients with pancreatic cancer have severely suppressed NK function. Both cytotoxicity and the ability to secrete the IFN-γ are suppressed in the patients. In addition, the numbers of peripheral blood mononuclear cells are also severely decreased in cancer patients. Interestingly, the percentages of both NK cells and monocyte are significantly increased whereas the percentage of CD3+ T cells and B cells is significantly decreased and the percentage of CD11b+ cells in increased. Furthermore, the majority of cytokines secreted by the NK cells or detected in the sera of the cancer patients are also severely decreased indicating a profound suppression of the immune function in cancer patients. Moreover, OC-modulated expansion of cancer patient NK cells is severely inhibited and the expanded patient NK cells mediated significantly low cytotoxicity and IFN-γ secretion. In cancer patients, both primary and expanded NK cells are defective in the function. Expansion of T cells as well as IFN-γ secretion is also decreased in cancer patients under different activation conditions. Cancer patients demonstrated high CD45RO and decreased CD62L indicating the increased activation in vivo. This is also evident for the increased percentage of CD8+ T cells and declined ratio of CD4/CD8 (FIGS. 35B and 35C). These unexpected results indicate that NK cells are very important in the preferential expansion of CD8+ T cells. In particular, OCs are important cells in the expansion of NK cells. The majority of T cells expanded by the NK cells are CD8+ T cells, and similar profile of CD8+ T cells expansion by the NK cells is seen both in healthy individuals and in cancer patients indicating that NK cells are indispensable for the expansion of CD8+ T cells. Although OCs decreased the ratio of CD4+ T cells to CD8+ T cells in both healthy individuals and cancer patients, the levels are substantially decreased in the presence of NK cells (FIG. 35E).

Interestingly, significant differences are observed between DC-modulated expansion of NK cells and OC-modulated NK cells expansion. When OC-modulated expansion resulted in the secondary expansion of NK cells modulated expansion of CD8+ T cells, DC-modulated expansion on NK cells resulted in secondary expansion of CD4+ T cells. There is larger increase in CD45RO and decrease in CD62L in T cells expanded by OC expanded NK cells than DC expanded NK cells indicating increased activation of T cells by the NK cells (FIG. 36J). High activation signals are necessary for the expansion of CD8+ T cells than CD4+ T cells. Indeed, OC-modulated expansion of CD8+ T cells resulted in gradual increase in the expansion of CD8+ T cells and in the decline of CD4+ T cells (FIG. 40B). Therefore, signals from both OCs and NK cells are important in activation of T cells toward CD8+ T cells. In addition, NK cells expanded by OCs have greater cytotoxic activity than those expanded by the DCs providing the mechanism for targeting of CD4+ T cells and sparing CD8+ T cells. In support of this observation, NK cells differentially targeted CD4+ T cells and CD8+ T cells. The IL-2+anti-CD16mAb activated NK cells are able to target CD4+ T cells indicating greater death receptor modulated induced cell death, since granular modulated cytotoxicity is inhibited by the anti-CD16mAb whereas increase in TNFRα and potentially Fas ligand and Apo2 ligand are observed. Accordingly, significant increase is observed in Fas ligand and TNFα secretion by OC-expanded NK cells and the levels exceeded significantly for those seen induced by the both CD4+ T cells and CD8+ T cells.

It is demonstrated herein that injection of OC-expanded NK cells to tumor-bearing hu-BLT mice increased the numbers of CD8+ T cells in bone marrow, spleen, and peripheral blood resulting in the increased levels of NK cell-mediated cytotoxicity as well as increased secretion of IFN-γ (FIGS. 38B-38J). Increased levels of IFN-γ, IL6, ITAC were also observed in the sera of tumor-bearing hu-BLT mice injected with OC-expanded NK cells (FIG. 39).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

### REFERENCES

1. Von Hoff, D. D., et al., Increased Survival in Pancreatic Cancer with nab-Paclitaxel plus Gemcitabine. New England Journal of Medicine, 2013. 369(18): p. 1691-1703.
2. Burris, H. A., et al., Improvements in survival and clinical benefit with gemcitabine as first-line therapy for patients with advanced pancreas cancer: A randomized trial. Journal of Clinical Oncology, 1997. 15(6): p. 2403-2413.
3. Philip, P. A., et al., Phase III Study Comparing Gemcitabine Plus Cetuximab Versus Gemcitabine in Patients With Advanced Pancreatic Adenocarcinoma: Southwest Oncology Group-Directed Intergroup Trial 50205. Journal of Clinical Oncology, 2010. 28(22): p. 3605-3610.
4. Li, C., et al., Identification of pancreatic cancer stem cells. Cancer Research, 2007. 67(3): p. 1030-1037.
5. Li, C., C. J. Lee, and D. M. Simeone, Identification of human pancreatic cancer stem cells. Methods Mol Biol, 2009. 568: p. 161-73.
6. Bao, B., et al., Pancreatic cancer stem-like cells display aggressive behavior mediated via activation of FoxQ1. J Biol Chem, 2014. 289(21): p. 14520-33.
7. Du, Z., et al., Pancreatic Cancer Cells Resistant to Chemoradiotherapy Rich in “Stem-Cell-Like” Tumor Cells. Digestive Diseases and Sciences, 2011. 56(3): p. 741-750.
8. Shah, A. N., et al., Development and characterization of gemcitabine-resistant pancreatic tumor cells. Annals of Surgical Oncology, 2007. 14(12): p. 3629-3637.
9. Wang, Z., et al., Acquisition of epithelial-mesenchymal transition phenotype of gemcitabine-resistant pancreatic cancer cells is linked with activation of notch signaling pathway. Proceedings of the American Association for Cancer Research Annual Meeting, 2009. 50: p. 765-766.
10. Ryschich, E., et al., Control of T-Cell-mediated immune response by HLA class I in human pancreatic carcinoma. Clinical Cancer Research, 2005. 11(2): p. 498-504.
11. Pandha, H., et al., Loss of expression of antigen-presenting molecules in human pancreatic cancer and pancreatic cancer cell lines. Clinical and Experimental Immunology, 2007. 148(1): p. 127-135.
12. Jewett, A., Y.-G. Man, and H.-C. Tseng, Dual Functions of Natural Killer Cells in Selection and Differentiation of Stem Cells; Role in Regulation of Inflammation and Regeneration of Tissues. Journal of Cancer, 2013. 4(1): p. 12-24.
13. Jewett, A., et al., Strategies to rescue mesenchymal stem cells (MSCs) and dental pulp stem cells (DPSCs) from NK cell mediated cytotoxicity. PLoS One, 2010. 5(3): p. e9874.
14. Jewett, A., et al., Coengagement of CD16 and CD94 receptors mediates secretion of chemokines and induces apoptotic death of naive natural killer cells. Clinical Cancer Research, 2006. 12(7): p. 1994-2003.
15. Aggarwal, S. and M. F. Pittenger, Human mesenchymal stem cells modulate allogeneic immune cell responses. Blood, 2005. 105(4): p. 1815-22.
16. Selmani, Z., et al., Human leukocyte antigen-G5 secretion by human mesenchymal stem cells is required to suppress T lymphocyte and natural killer function and to induce CD4(+)CD25(high)FOXP3(+) regulatory T cells. Stem Cells, 2008. 26(1): p. 212-222.
17. Spaggiari, G. M., et al., Mesenchymal stem cells inhibit natural killer-cell proliferation, cytotoxicity, and cytokine production: role of indoleamine 2,3-dioxygenase and prostaglandin E2. Blood, 2008. 111(3): p. 1327-1333.
18. Westin, J. R., et al., Safety and activity of PD1 blockade by pidilizumab in combination with rituximab in patients with relapsed follicular lymphoma: a single group, open-label, phase 2 trial. Lancet Oncol, 2014. 15(1): p. 69-77.
19. Lee, J. H., et al., Circulating tumour DNA predicts response to anti-PD1 antibodies in metastatic melanoma. Ann Oncol, 2017. 28(5): p. 1130-1136.
20. Kitayama, J., et al., FUNCTIONAL-ANALYSIS OF TCR-GAMMA-DELTA+ T-CELLS IN TUMOR-INFILTRATING LYMPHOCYTES (TIL) OF HUMAN PANCREATIC-CANCER. Clinical and Experimental Immunology, 1993. 93(3): p. 442-447.
21. Degrate, L., et al., Interleukin-2 immunotherapy action on innate immunity cells in peripheral blood and tumoral tissue of pancreatic adenocarcinoma patients. Langenbecks Archives of Surgery, 2009. 394(1): p. 115-121.
22. Aparicio-Pages, M. N., et al., Natural killer cell activity in patients with adenocarcinoma in the upper gastrointestinal tract. Journal of clinical & laboratory immunology, 1991. 35(1): p. 27-32.
23. Duan, X., et al., Clinical significance of the immunostimulatory MHC class I chain-related molecule A and NKG2D receptor on NK cells in pancreatic cancer. Medical Oncology, 2011. 28(2): p. 466-474.
24. Peng, Y.-P., et al., Comprehensive analysis of the percentage of surface receptors and cytotoxic granules positive natural killer cells in patients with pancreatic cancer, gastric cancer, and colorectal cancer. Journal of Translational Medicine, 2013. 11.
25. Kaur, K., et al., Suppression of Gingival NK Cells in Precancerous and Cancerous Stages of Pancreatic Cancer in KC and BLT-Humanized Mice. Frontiers in Immunology, 2017. 8(1606).
26. Ruggeri, L., et al., Effectiveness of Donor Natural Killer Cell Alloreactivity in Mismatched Hematopoietic Transplants. Science, 2002. 295(5562): p. 2097-2100.
27. Venstrom, J. M., et al., HLA-C-dependent prevention of leukemia relapse by donor activating KIR2DS1. N Engl J Med, 2012. 367(9): p. 805-16.
28. Iliopoulou, E. G., et al., A phase I trial of adoptive transfer of allogeneic natural killer cells in patients with advanced non-small cell lung cancer. Cancer Immunol Immunother, 2010. 59(12): p. 1781-9.
29. Miller, J. S., et al., Successful adoptive transfer and in vivo expansion of human haploidentical NK cells in patients with cancer. Blood, 2005. 105(8): p. 3051-7.
30. Re, F., et al., Killer cell Ig-like receptors ligand-mismatched, alloreactive natural killer cells lyse primary solid tumors. Cancer, 2006. 107(3): p. 640-8.
31. Geller, M. A., et al., A phase II study of allogeneic natural killer cell therapy to treat patients with recurrent ovarian and breast cancer. Cytotherapy, 2011. 13(1): p. 98-107.
32. Kozlowska, A. K., et al., Differentiation by NK cells is a prerequisite for effective targeting of cancer stem cells/poorly differentiated tumors by chemopreventive and chemotherapeutic drugs. J Cancer, 2017. 8(4): p. 537-554.
33. Sipos, B., et al., A comprehensive characterization of pancreatic ductal carcinoma cell lines: towards the establishment of an in vitro research platform. Virchows Arch, 2003. 442(5): p. 444-52.
34. Kozlowska, A. K., et al., Resistance to cytotoxicity and sustained release of interleukin-6 and interleukin-8 in the presence of decreased interferon-gamma after differentiation of glioblastoma by human natural killer cells. Cancer Immunol Immunother, 2016. 65(9): p. 1085-97.
35. Kaur, K., et al., Natural killer cells target and differentiate cancer stem-like cells/undifferentiated tumors: strategies to optimize their growth and expansion for effective cancer immunotherapy. Curr Opin Immunol, 2018. 51: p. 170-180.
36. Jewett, A., et al., NK cells shape pancreatic and oral tumor microenvironments; role in inhibition of tumor growth and metastasis. Semin Cancer Biol, 2018.
37. Paranjpe, A., et al., N-acetylcysteine protects dental pulp stromal cells from HEMA-induced apoptosis by inducing differentiation of the cells. Free Radic Biol Med, 2007. 43(10): p. 1394-408.
38. Tseng, H. C., N. Cacalano, and A. Jewett, Split anergized Natural Killer cells halt inflammation by inducing stem cell differentiation, resistance to NK cell cytotoxicity and prevention of cytokine and chemokine secretion. Oncotarget, 2015. 6(11): p. 8947-59.
39. Jewett, A., Y. G. Man, and H. C. Tseng, Dual functions of natural killer cells in selection and differentiation of stem cells; role in regulation of inflammation and regeneration of tissues. J Cancer, 2013. 4(1): p. 12-24.
40. Jewett, A. and H. C. Tseng, Potential rescue, survival and differentiation of cancer stem cells and primary non-transformed stem cells by monocyte-induced split anergy in natural killer cells. Cancer Immunol Immunother, 2012. 61(2): p. 265-74.
41. Paranjpe, A., et al., N-acetyl cysteine mediates protection from 2-hydroxyethyl methacrylate induced apoptosis via nuclear factor kappa B-dependent and independent pathways: potential involvement of JNK. Toxicol Sci, 2009. 108(2): p. 356-66.
42. Routy, B., et al., Gut microbiome influences efficacy of PD-1-based immunotherapy against epithelial tumors. Science, 2017.
43. Vivier E, Raulet D H, Moretta A, Caligiuri M A, Zitvogel L, Lanier L L, Yokoyama W M, Ugolini S: Innate or adaptive immunity? The example of natural killer cells. Science (New York, N.Y.) 2011, 331(6013):44-49.
44. Shaw S Y, Tran K, Castoreno A B, Peloquin J M, Lassen K G, Khor B, Aldrich L N, Tan P H, Graham D B, Kuballa P et al: Selective modulation of autophagy, innate immunity, and adaptive immunity by small molecules. ACS chemical biology 2013, 8(12):2724-2733.
45. Cooper M A, Fehniger T A, Caligiuri M A: The biology of human natural killer-cell subsets. Trends Immunol 2001, 22(11):633-640.
46. Farag S S, Caligiuri M A: Human natural killer cell development and biology. Blood reviews, 20(3):123-137.
47. Sun H, Sun C, Tian Z, Xiao W: NK cells in immunotolerant organs. Cellular & molecular immunology 2013, 10(3):202-212.
48. Caras I, Grigorescu A, Stavaru C, Radu D L, Mogos I, Szegli G, Salageanu A: Evidence for immune defects in breast and lung cancer patients. Cancer Immunol Immunother 2004, 53(12):1146-1152.
49. Kaur K, Cook J, Park S H, Topchyan P, Kozlowska A, Ohanian N, Fang C, Nishimura I, Jewett A: Novel Strategy to Expand Super-Charged NK Cells with Significant Potential to Lyse and Differentiate Cancer Stem Cells: Differences in NK Expansion and Function between Healthy and Cancer Patients. Frontiers in immunology 2017, 8:297.
50. Speiser D E, Ho P C, Verdeil G: Regulatory circuits of T cell function in cancer. Nature reviews Immunology 2016, 16(10):599-611.
51. Igarashi T, Wynberg J, Srinivasan R, Becknell B, McCoy J P, Jr., Takahashi Y, Suffredini D A, Linehan W M, Caligiuri M A, Childs R W: Enhanced cytotoxicity of allogeneic NK cells with killer immunoglobulin-like receptor ligand incompatibility against melanoma and renal cell carcinoma cells. Blood 2004, 104(1):170-177.
52. White D, Jones D B, Cooke T, Kirkham N: Natural killer (NK) activity in peripheral blood lymphocytes of patients with benign and malignant breast disease. Br J Cancer 1982, 46(4):611-616.
53. Jewett A, Tseng H C: Tumor induced inactivation of natural killer cell cytotoxic function; implication in growth, expansion and differentiation of cancer stem cells. J Cancer 2011, 2:443-457.
54. Santos M F, Mannam V K, Craft B S, Puneky L V, Sheehan N T, Lewis R E, Cruse J M: Comparative analysis of innate immune system function in metastatic breast, colorectal, and prostate cancer patients with circulating tumor cells. Experimental and molecular pathology 2014, 96(3):367-374.
55. Matsumoto Y, Tsujimoto H, Ono S, Shinomiya N, Miyazaki H, Hiraki S, Takahata R, Yoshida K, Saitoh D, Yamori T et al: Abdominal Infection Suppresses the Number and Activity of Intrahepatic Natural Killer Cells and Promotes Tumor Growth in a Murine Liver Metastasis Model. Annals of surgical oncology 2016, 23 Suppl 2:S257-265.
56. Baskic D, Vujanovic L, Arsenijevic N, Whiteside T L, Myers E N, Vujanovic N L: Suppression of natural killer-cell and dendritic-cell apoptotic tumoricidal activity in patients with head and neck cancer. Head & neck 2013, 35(3):388-398.
57. Mickel R A, Kessler D J, Taylor J M, Lichtenstein A: Natural killer cell cytotoxicity in the peripheral blood, cervical lymph nodes, and tumor of head and neck cancer patients. Cancer Res 1988, 48(17):5017-5022.
58. Ghoneum M, Gill G, Perry L: Natural killer cell activity in patients with carcinoma of the larynx and hypopharynx. The Laryngoscope 1986, 96(11):1300.
59. Tartter P I, Steinberg B, Barron D M, Martinelli G: The prognostic significance of natural killer cytotoxicity in patients with colorectal cancer. Archives of surgery (Chicago, Ill.: 1960) 1987, 122(11):1264-1268.
60. Izawa S, Kono K, Mimura K, Kawaguchi Y, Watanabe M, Maruyama T, Fujii H: H₂O₂ production within tumor microenvironment inversely correlated with infiltration of CD56dim NK cells in gastric and esophageal cancer: possible mechanisms of NK cell dysfunction. Cancer Immunology, Immunotherapy 2011, 60(12):1801-1810.
61. Nolibe D, Poupon M F: Enhancement of pulmonary metastases induced by decreased lung natural killer cell activity. Journal of the National Cancer Institute 1986, 77(1):99-103.
62. Imai K, Matsuyama S, Miyake S, Suga K, Nakachi K: Natural cytotoxic activity of peripheral-blood lymphocytes and cancer incidence: an 11-year follow-up study of a general population. Lancet 2000, 356(9244):1795-1799.
63. Bruno A, Ferlazzo G, Albini A, Noonan D M: A think tank of TINK/TANKs: tumor-infiltrating/tumor-associated natural killer cells in tumor progression and angiogenesis. Journal of the National Cancer Institute 2014, 106(8):dju200.
64. Vitale M, Cantoni C, Pietra G, Mingari M C, Moretta L: Effect of tumor cells and tumor microenvironment on NK-cell function. European journal of immunology 2014, 44(6): 1582-1592.
65. Mirjacic Martinovic K M, Babovic N, Dzodic R R, Jurisic V B, Tanic N T, Konjevic G M: Decreased expression of NKG2D, NKp46, DNAM-1 receptors, and intracellular perforin and STAT-1 effector molecules in NK cells and their dim and bright subsets in metastatic melanoma patients. Melanoma research 2014, 24(4):295-304.
66. Gallois A, Silva I, Osman I, Bhardwaj N: Reversal of natural killer cell exhaustion by TIM-3 blockade. Oncoimmunology 2014, 3(12):e946365.
67. Hersey P, Edwards A, Honeyman M, McCarthy W H: Low natural-killer-cell activity in familial melanoma patients and their relatives. British journal of cancer 1979, 40(1):113-122.
68. Gubbels J A, Felder M, Horibata S, Belisle J A, Kapur A, Holden H, Petrie S, Migneault M, Rancourt C, Connor J P et al: MUC16 provides immune protection by inhibiting synapse formation between NK and ovarian tumor cells. Molecular cancer 2010, 9:11.
69. Balsamo M, Scordamaglia F, Pietra G, Manzini C, Cantoni C, Boitano M, Queirolo P, Vermi W, Facchetti F, Moretta A et al: Melanoma-associated fibroblasts modulate NK cell phenotype and antitumor cytotoxicity. Proceedings of the National Academy of Sciences of the U.S. Pat. No. 2,009,106(49):20847-20852.
70. Castriconi R, Cantoni C, Della Chiesa M, Vitale M, Marcenaro E, Conte R, Biassoni R, Bottino C, Moretta L, Moretta A: Transforming growth factor beta 1 inhibits expression of NKp30 and NKG2D receptors: consequences for the NK-mediated killing of dendritic cells. Proceedings of the National Academy of Sciences of the United States of America 2003, 100(7):4120-4125.
71. Pietra G, Manzini C, Rivara S, Vitale M, Cantoni C, Petretto A, Balsamo M, Conte R, Benelli R, Minghelli S et al: Melanoma cells inhibit natural killer cell function by modulating the expression of activating receptors and cytolytic activity. Cancer research 2012, 72(6):1407-1415.
72. Hanahan D, Weinberg R A: Hallmarks of cancer: the next generation. Cell 2011, 144(5):646-674.
73. Smyth M J, Hayakawa Y, Takeda K, Yagita H: New aspects of natural-killer-cell surveillance and therapy of cancer. Nature reviews Cancer 2002, 2(11):850-861.
74. Gross E, Sunwoo J B, Bui J D: Cancer immunosurveillance and immunoediting by natural killer cells. Cancer journal (Sudbury, Mass.) 2013, 19(6):483-489.
75. Krockenberger M, Dombrowski Y, Weidler C, Ossadnik M, Honig A, Hausler S, Voigt H, Becker J C, Leng L, Steinle A et al: Macrophage migration inhibitory factor contributes to the immune escape of ovarian cancer by down-regulating NKG2D. Journal of immunology (Baltimore, Md.: 1950) 2008, 180(11):7338-7348.
76. Burke S, Lakshmikanth T, Colucci F, Carbone E: New views on natural killer cell-based immunotherapy for melanoma treatment. Trends in immunology 2010, 31(9):339-345.
77. Larsen S K, Gao Y, Basse P H: NK cells in the tumor microenvironment. Critical reviews in oncogenesis 2014, 19(1-2):91-105.
78. Harning R, Koo G C, Szalay J: Regulation of the metastasis of murine ocular melanoma by natural killer cells. Investigative ophthalmology & visual science 1989, 30(9):1909-1915.
79. Coca S, Perez-Piqueras J, Martinez D, Colmenarejo A, Saez M A, Vallejo C, Martos J A, Moreno M: The prognostic significance of intratumoral natural killer cells in patients with colorectal carcinoma. Cancer 1997, 79(12):2320-2328.
80. Pant H, Hughes A, Miljkovic D, Schembri M, Wormald P, Macardle P, Grose R, Zola H, Krumbiegel D: Accumulation of effector memory CD8+ T cells in nasal polyps. American journal of rhinology & allergy 2013, 27(5):e117-126.
81. Zanetti M: Tapping CD4 T cells for cancer immunotherapy: the choice of personalized genomics. J Immunol 2015, 194(5):2049-2056.
82. Seo A N, Lee H J, Kim E J, Kim H J, Jang M R, Lee H E, Kim Y J, Kim J H, Park S Y: Tumour-infiltrating CD8+ lymphocytes as an independent predictive factor for pathological complete response to primary systemic therapy in breast cancer. Br J Cancer 2013, 109(10):2705-2713.
83. Hadrup S, Donia M, Thor Straten P: Effector CD4 and CD8 T cells and their role in the tumor microenvironment. Cancer microenvironment: official journal of the International Cancer Microenvironment Society 2013, 6(2):123-133.
84. Kim S T, Jeong H, Woo O H, Seo J H, Kim A, Lee E S, Shin S W, Kim Y H, Kim J S, Park K H: Tumor-infiltrating lymphocytes, tumor characteristics, and recurrence in patients with early breast cancer. American journal of clinical oncology 2013, 36(3):224-231.
85. Shah W, Yan X, Jing L, Zhou Y, Chen H, Wang Y: A reversed CD4/CD8 ratio of tumor-infiltrating lymphocytes and a high percentage of CD4(+)FOXP3(+) regulatory T cells are significantly associated with clinical outcome in squamous cell carcinoma of the cervix. Cellular & molecular immunology 2011, 8(1):59-66.
86. Robbins S H, Bessou G, Cornillon A, Zucchini N, Rupp B, Ruzsics Z, Sacher T, Tomasello E, Vivier E, Koszinowski U H et al: Natural killer cells promote early CD8 T cell responses against cytomegalovirus. PLoS pathogens 2007, 3(8):e123.
87. Wodarz D, Sierro S, Klenerman P: Dynamics of killer T cell inflation in viral infections. Journal of the Royal Society, Interface 2007, 4(14):533-543.
88. Wu Z, Xu Y: IL-15R alpha-IgG1-Fc enhances IL-2 and IL-15 anti-tumor action through NK and CD8+ T cells proliferation and activation. Journal of molecular cell biology 2010, 2(4):217-222.
89. Tomala J, Chmelova H, Mrkvan T, Rihova B, Kovar M: In vivo expansion of activated naive CD8+ T cells and NK cells driven by complexes of IL-2 and anti-IL-2 monoclonal antibody as novel approach of cancer immunotherapy. J Immunol 2009, 183(8):4904-4912.
90. Tanaka J, Toubai T, Miura Y, Tsutsumi Y, Kato N, Umehara S, Toyoshima N, Ohta S, Asaka M, Imamura M: Differential expression of natural killer cell receptors (CD94/NKG2A) on T cells by the stimulation of G-CSF-mobilized peripheral blood mononuclear cells with anti-CD3 monoclonal antibody and cytokines: a study in stem cell donors. Transplantation proceedings 2004, 36(8):2511-2512.
91. Tseng H C, Arasteh A, Paranjpe A, Teruel A, Yang W, Behel A, Alva J A, Walter G, Head C, Ishikawa T O et al: Increased lysis of stem cells but not their differentiated cells by natural killer cells; de-differentiation or reprogramming activates NK cells. PloS one 2010, 5(7):e11590.
92. Tseng H C, Bui V, Man Y G, Cacalano N, Jewett A: Induction of Split Anergy Conditions Natural Killer Cells to Promote Differentiation of Stem Cells through Cell-Cell Contact and Secreted Factors. Frontiers in immunology 2014, 5:269.
93. Tseng H C, Inagaki A, Bui V T, Cacalano N, Kasahara N, Man Y G, Jewett A: Differential Targeting of Stem Cells and Differentiated Glioblastomas by NK Cells. Journal of Cancer 2015, 6(9):866-876.
94. Bui V T, Tseng H-C, Maung P O, Kozlowska A, Mann K, Topchyan P, Jewett A: Augmented IFN-γ and TNF-α Induced by Probiotic Bacteria in NK Cells Mediate Differentiation of Stem-Like Tumors Leading to Inhibition of Tumor Growth and Reduction in Inflammatory Cytokine Release; Regulation by IL-10. Frontiers in immunology 2015, 6.
95. Jewett A, Bonavida B: Target-induced inactivation and cell death by apoptosis in a subset of human NK cells. Journal of immunology (Baltimore, Md.: 1950) 1996, 156(3):907-915.
96. Tseng H C, Kanayama K, Kaur K, Park S H, Park S, Kozlowska A, Sun S, McKenna C E, Nishimura I, Jewett A: Bisphosphonate-induced differential modulation of immune cell function in gingiva and bone marrow in vivo: Role in osteoclast-mediated NK cell activation. Oncotarget 2015, 6(24):20002-20025.
97. Bui V T, Tseng H C, Kozlowska A, Maung P O, Kaur K, Topchyan P, Jewett A: Augmented IFN-gamma and TNF-alpha Induced by Probiotic Bacteria in NK Cells Mediate Differentiation of Stem-Like Tumors Leading to Inhibition of Tumor Growth and Reduction in Inflammatory Cytokine Release; Regulation by IL-10. Frontiers in immunology 2015, 6:576.
98. Shimizu S, Hong P, Arumugam B, Pokomo L, Boyer J, Koizumi N, Kittipongdaja P, Chen A, Bristol G, Galic Z et al: A highly efficient short hairpin RNA potently down-regulates CCR5 expression in systemic lymphoid organs in the hu-BLT mouse model. Blood 2010, 115(8):1534-1544.
99. Vatakis D N, Koya R C, Nixon C C, Wei L, Kim S G, Avancena P, Bristol G, Baltimore D, Kohn D B, Ribas A et al: Antitumor activity from antigen-specific CD8 T cells generated in vivo from genetically engineered human hematopoietic stem cells. Proceedings of the National Academy of Sciences of the United States of America 2011, 108(51):E1408-1416.
100. Kozlowska A K, Kaur K, Topchyan P, Jewett A: Adoptive transfer of osteoclast-expanded natural killer cells for immunotherapy targeting cancer stem-like cells in humanized mice. Cancer immunology, immunotherapy: CII 2016.
101. Jewett A, Cavalcanti M, Bonavida B: Pivotal role of endogenous TNF-alpha in the induction of functional inactivation and apoptosis in NK cells. Journal of immunology (Baltimore, Md.: 1950) 1997, 159(10):4815-4822.
102. Jewett A, Bonavida B: Interferon-alpha activates cytotoxic function but inhibits interleukin-2-mediated proliferation and tumor necrosis factor-alpha secretion by immature human natural killer cells. J Clin Immunol 1995, 15(1):35-44.
103. Jewett A, Wang M Y, Teruel A, Poupak Z, Bostanian Z, Park N H: Cytokine dependent inverse regulation of CD54 (ICAM1) and major histocompatibility complex class I antigens by nuclear factor kappaB in HEp2 tumor cell line: effect on the function of natural killer cells. Human immunology 2003, 64(5):505-520.
104. Kaur K, Chang H H, Topchyan P, Cook J M, Barkhordarian A, Eibl G, Jewett A: Deficiencies in Natural Killer Cell Numbers, Expansion, and Function at the Pre-Neoplastic Stage of Pancreatic Cancer by KRAS Mutation in the Pancreas of Obese Mice. Frontiers in immunology 2018, 9:1229.
105. Gattinoni L, Klebanoff C A, Restifo N P: Paths to stemness: building the ultimate antitumour T cell. Nature reviews Cancer 2012, 12(10):671-684.
106. Paiardini M, Cervasi B, Albrecht H, Muthukumar A, Dunham R, Gordon S, Radziewicz H, Piedimonte G, Magnani M, Montroni M et al: Loss of CD127 Expression Defines an Expansion of Effector CD8<sup>+</sup> T Cells in HIV-Infected Individuals. The Journal of Immunology 2005, 174(5):2900-2909.
107. Drennan S, Stafford N D, Greenman J, Green V L: Increased frequency and suppressive activity of CD127(low/-) regulatory T cells in the peripheral circulation of patients with head and neck squamous cell carcinoma are associated with advanced stage and nodal involvement. Immunology 2013, 140(3):335-343.

We claim:

1. A method of treating a subject afflicted with a cancer comprising administering to the subject an immunological composition, wherein the immunological composition comprises at least two cell types selected from:
(a) allogeneic primary NK cells,
(b) allogeneic super-charged NK cells,
(c) autologous super-charged NK cell expanded CD8+ T cells, and
(d) allogeneic super-charged NK cell expanded autologous CD8+ T cells.

2. The method of claim 1, wherein the immunological composition comprises three cell types.

3. The method of claim 1, wherein the immunological composition comprises four cell types.

4. The method of any preceding claim, wherein the NK cells of the subject show one or more reduced activities selected from: (a) cytokine secretion, optionally wherein the cytokine is IFN-γ, (b) cytotoxicity, (c) expansion of CD8+ T cells, (d) differentiation of stem-like/poorly differentiated tumor cells, and (e) ADCC activity.

5. The method of any preceding claim, wherein the immunological composition is administered in a pharmaceutically acceptable formulation.

6. The method of any preceding claim, further comprising administering to the subject an antibody against at least one surface protein that is highly expressed on cancer cells.

7. The method of claim 6, wherein the antibody binds MICA/MICB.

8. The method of claim 6 or 7, wherein the antibody is administered in an amount sufficient to induce ADCC.

9. The method of any preceding claim, further comprising activating NK cells by inducing or enhancing secretion of IFN-γ in the NK cells.

10. The method of claim 9, wherein activating NK cells comprises administering to the subject one or more additional agents that enhance secretion of IFN-γ by the NK cells.

11. The method of claim 10, wherein the one or more additional agents are selected from IL-2, anti-CD16 antibody, anti-CD3 antibody, anti-CD28 antibody, Mekabu, and a composition comprising at least one bacterial strain.

12. The method of claim 11, wherein the composition comprises at least one bacterial strain selected from: Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus, optionally wherein the at least one bacterial strain is either alive or sonicated.

13. The method of claim 12, wherein the composition comprises AJ2 bacteria.

14. The method of claim 11, wherein the one or more additional agents are Mekabu and AJ2 bacteria.

15. The method of any preceding claim, further comprising administering to the subject at least one additional immunotherapy and/or cancer therapy.

16. The method of claim 15, wherein the immunotherapy and/or cancer therapy is administered before, after, or concurrently with the immunological composition.

17. The method of claim 15 or 16, wherein the at least one additional immunotherapy inhibits an immune checkpoint.

18. The method of claim 17, wherein the immune checkpoint is selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR.

19. The method of claim 18, wherein the immune checkpoint is selected from CTLA-4, PD-1, PD-L1, and PD-L2.

20. The method of claim 15, wherein the cancer therapy is selected from radiation, a radiosensitizer, a chemotherapy, interferon, and an interferon-inducing agent.

21. The method of claim 20, wherein the cancer therapy is a chemotherapy, optionally wherein the chemotherapy is paclitaxel and/or cisplatin.

22. The method any preceding claim, further comprising administering to the subject an agent that induces differentiation of poorly differentiated cancer cells, optionally wherein the agent is N-acetylcysteine (NAC).

23. The method of any preceding claim, wherein the cancer is pancreatic cancer, or oral cancer, optionally wherein the oral cancer is oral squamous carcinoma.

24. The method of any preceding claim, wherein the cancer is highly differentiated.

25. The method of any preceding claim, wherein the cancer is stem-like/poorly differentiated.

26. The method of any preceding claim, wherein the subject is a mammal.

27. The method of claim 26, wherein the mammal is a mouse or a human.

28. The method of claim 27, wherein the mammal is a human.

29. A method of killing or inhibiting proliferation of cancer cells comprising contacting the cancer cells with an immunological composition, wherein the immunological composition comprises at least two cell types selected from:
(a) allogeneic primary NK cells,
(b) allogeneic super-charged NK cells,
(c) autologous super-charged NK cell expanded CD8+ T cells, and
(d) allogeneic super-charged NK cell expanded autologous CD8+ T cells.

30. The method of claim 29, wherein the immunological composition comprises three cell types.

31. The method of claim 29, wherein the immunological composition comprises four cell types.

32. The method of any one of claims 29-31, wherein the immunological composition is in a pharmaceutically acceptable formulation.

33. The method of any one of claims 29-32, further comprising contacting the cancer cells with an antibody against at least one surface protein that is highly expressed on cancer cells.

34. The method of claim 33, wherein the antibody binds MICA/MICB.

35. The method of claim 33 or 34, wherein the antibody is in an amount sufficient to induce ADCC.

36. The method of any one of claims 29-35, further comprising activating NK cells by inducing or enhancing secretion of IFN-γ in the NK cells.

37. The method of claim 36, wherein activating NK cells comprises contacting the NK cells with one or more additional agents that enhance secretion of IFN-γ by the NK cells.

38. The method of claim 37, wherein the one or more additional agents are selected from IL-2, anti-CD16 antibody, anti-CD3 antibody, anti-CD28 antibody, Mekabu, and a composition comprising at least one bacterial strain.

39. The method of claim 38, wherein the composition comprises at least one bacterial strain selected from: Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus, optionally wherein the at least one bacterial strain is either alive or sonicated.

40. The method of claim 39, wherein the composition comprises AJ2 bacteria.

41. The method of claim 38, wherein the one or more additional agents are Mekabu and AJ2 bacteria.

42. The method of any one of claims 29-41, further comprising contacting the cancer cells with at least one additional immunotherapy and/or cancer therapy.

43. The method of claim 42, wherein the at least one additional immunotherapy and/or cancer therapy is added before, after, or concurrently with the immunological composition.

44. The method of claim 42 or 43, wherein the at least one additional immunotherapy inhibits an immune checkpoint.

45. The method of claim 44, wherein the immune checkpoint is selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, IDO, CD39, CD73 and A2aR.

46. The method of claim 45, wherein the immune checkpoint is selected from CTLA-4, PD-1, PD-L1, and PD-L2.

47. The method of claim 42, wherein the cancer therapy is selected from radiation, a radiosensitizer, a chemotherapy, interferon, and an interferon-inducing agent.

48. The method of claim 47, wherein the cancer therapy is a chemotherapy, optionally wherein the chemotherapy is a paclitaxel and/or cisplatin.

49. The method of any preceding claim, further comprising contacting the cancer cells with an agent that induces differentiation of poorly differentiated cancer cells, optionally wherein the agent is N-acetylcysteine (NAC).

50. The method of any one of claims 29-49, wherein the cancer is pancreatic cancer, or oral cancer, optionally wherein the oral cancer is oral squamous carcinoma.

51. The method of any one of claims 29-50, wherein the cancer is highly differentiated.

52. The method of any one of claims 29-50, wherein the cancer is stem-like/poorly differentiated.

53. The method of any one of claims 29-52, wherein the subject is a mammal.

54. The method of claim 53, wherein the mammal is a mouse or a human.

55. The method of claim 54, wherein the mammal is a human.

56. An immunological composition capable of eliciting an immune response in a subject, comprising at least two cell types selected from:
(a) allogeneic primary NK cells,
(b) allogeneic super-charged NK cells,
(c) autologous super-charged NK cell expanded CD8+ T cells, and
(d) allogeneic super-charged NK cell expanded autologous CD8+ T cells.

57. The immunological composition of claim 56, wherein the immunological composition comprises three cell types.

58. The immunological composition of claim 56, wherein the immunological composition comprises four cell types.

59. The immunological composition of any one of claims 56-58, wherein the immunological composition is in a pharmaceutically acceptable formulation.

60. The immunological composition of any one of claims 56-59, further comprising an antibody against at least one surface protein that is highly expressed on cancer cells.

61. The immunological composition of claim 60, wherein the antibody binds MICA/MICB.

62. The immunological composition of claim 60 or 61, wherein the antibody is present in an amount sufficient to induce ADCC when administered to a subject.

63. The immunological composition of any one of claims 56-62, further comprising one or more additional agents that enhance secretion of IFN-γ by the NK cells.

64. The immunological composition of claim 63, wherein the one or more additional agents are selected from IL-2, anti-CD16 antibody, anti-CD3 antibody, anti-CD28 antibody, Mekabu, and a composition comprising at least one bacterial strain.

65. The immunological composition of claim 64, wherein the composition comprises at least one bacterial strain selected from: Streptococcus thermophiles, Bifidobacterium longum, Bifidobacterium breve, Bifidobacterium infantis, Lactobacillus acidophilus, Lactobacillus plantarum, Lactobacillus paracasei, KE99, and Lactobacillus bulgaricus, optionally wherein the at least one bacterial strain is either alive or sonicated.

66. The immunological composition of claim 65, wherein the composition comprises AJ2 bacteria.

67. The immunological composition of claim 64, wherein the one or more additional agents are Mekabu and AJ2 bacteria.

68. The immunological composition of any one of claims 56-67, further comprising at least one additional immunotherapy and/or cancer therapy.

69. The immunological composition of claim 68, wherein the at least one additional immunotherapy inhibits an immune checkpoint.

70. The immunological composition of claim 69, wherein the immune checkpoint is selected from CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRP, CD47, CD48, 2B4 (CD244), B7.1, B7.2, ILT-2, ILT-4, TIGIT, HHLA2, butyrophilins, DO, CD39, CD73 and A2aR.

71. The immunological composition of claim 70, wherein the immune checkpoint is selected from CTLA-4, PD-1, PD-L1, and PD-L2.

72. The immunological composition of claim 68, wherein the cancer therapy is selected from a radiosensitizer, a chemotherapy, interferon, and an interferon-inducing agent.

73. The immunological composition of claim 72, wherein the cancer therapy is chemotherapy, optionally wherein the chemotherapy is paclitaxel and/or cisplatin.

74. The immunological composition of any one of claims 56-73, further comprising an agent that induces differentiation of poorly differentiated cancer cells, optionally wherein the agent is N-acetylcysteine (NAC).
